(19) United States
(12) Patent Application Publication
(10) Pub. No.: US 2009/0246769 A1
(43) Pub. Date: Oct. 1, 2009

(54) MOLECULAR DIAGNOSIS OF OVARIAN CANCERS

(75) Inventors: Takaaki SATO, Tokyo (JP); Tetsuyoshi Sugita, Matsudo-shi (JP); Atsuhiko Toyama, Tokyo (JP); Takashi Shimada, Tsukuba-shi (JP); Daisuke Aoki, Tokyo (JP); Atsushi Suzuki, Tokyo (JP); Nobuyuki Susumu, Tokyo (JP); Hiroyuki Nomura, Tokyo (JP)

Correspondence Address: SUGHRUE MION, PLLC, 2100 PENNSYLVANIA AVENUE, N.W., SUITE 800, WASHINGTON, DC, 20037, US

(73) Assignees: SHIMADZU CORPORATION, Kyoto-shi, Kyoto (JP); KEIO UNIVERSITY, Minato-ku, Tokyo (JP)

(21) Appl. No.: 12/246,242

(22) Filed: Oct. 6, 2008

(30) Foreign Application Priority Data
Oct. 4, 2007 (JP) 2007-261395

(51) Int. Cl. C12Q 1/68; C12M 1/34; G01N 33/574

(52) U.S. Cl. 435/6; 435/287.2

(57) ABSTRACT

A molecular diagnosis system of ovarian cancers encompasses a detection device configured to obtain a detected value of an expression amount of an apolipoprotein A1 gene in ovarian tissue as a diagnosis subject, a storage device configured to store a normal value of the expression amount of the apolipoprotein A1 gene in normal ovarian tissue, and a determination mechanism configured to determine that the ovarian tissue as the diagnosis subject is clear cell adenocarcinoma when the detected value is lower than the normal value.

### CROSS REFERENCE TO RELATED APPLICATIONS AND INCORPORATION BY REFERENCE

This application claims benefit of priority under 35 USC 119 based on Japanese Patent Application No. P2007-261395, filed on Oct. 4, 2007, the entire contents of which are incorporated by reference herein.

### BACKGROUND OF THE INVENTION

1. Field of the Invention

The present invention relates to a diagnosis technique, and in particular, relates to a molecular diagnosis of ovarian cancers.

2. Description of the Related Art

Heretofore, there has been information on gene expression in ovarian cancer tissue. However, an amount of information on gene expression for each of tissue types of the ovarian cancer tissue has been extremely limited while the ovarian cancer tissue is classified into a variety of the tissue types. In recent years, as described in Japanese Unexamined Patent Application Publication No. P2001-517300, researches on medical diagnosis using a biochip in which a probe composed of deoxyribonucleic acid (DNA), ribonucleic acid (RNA) and the like is fixed to a substrate have been made actively. However, since there has hardly been such gene expression information for each of the tissue types of the ovarian cancer tissue, a diagnosis method for the ovarian cancer, which is based on the gene expression in ovarian tissue, has not been established yet.

### SUMMARY OF THE INVENTION

An object of the present invention is to provide a molecular diagnosis system of ovarian cancers, and a molecular diagnosis method of ovarian cancers, which are capable of diagnosing the ovarian cancers based on an expression amount of biomolecule.

A first aspect of the present invention inheres in a molecular diagnosis system of ovarian cancers encompassing (a) a detection device configured to obtain a detected value of an expression amount of an apolipoprotein A1 (ApoA1) gene in ovarian tissue as a diagnosis subject, (b) a storage device configured to store a normal value of the expression amount of the ApoA1 gene in normal ovarian tissue, and (c) a determination mechanism configured to determine that the ovarian tissue as the diagnosis subject is clear cell adenocarcinoma when the detected value is lower than the normal value.

A second aspect of the present invention inheres in a molecular diagnosis system of ovarian cancers encompassing (a) a detection device configured to obtain a detected value of an expression amount of an apolipoprotein E (ApoE) gene in ovarian tissue as a diagnosis subject, (b) a storage device configured to store a normal value of the expression amount of the ApoE gene in normal ovarian tissue, and (c) a determination mechanism configured to determine that the ovarian tissue as the diagnosis subject is epithelial ovarian carcinoma when the detected value is lower than the normal value.

A third aspect of the present invention inheres in a molecular diagnosis system of ovarian cancers encompassing (a) a detection device configured to obtain a detected value of an expression amount of an apolipoprotein J (ApoJ) gene in ovarian tissue as a diagnosis subject, (b) a storage device configured to store a normal value of the expression amount of the ApoJ gene in normal ovarian tissue, and (c) a determination mechanism configured to determine that the ovarian tissue as the diagnosis subject is mucinous adenocarcinoma or clear cell adenocarcinoma when the detected value is lower than the normal value.

A fourth aspect of the present invention inheres in a molecular diagnosis system of ovarian cancers encompassing (a) a detection device configured to obtain a detected value of an expression amount of a homo sapiens aldo-keto reductase family 1 member B10 (ARL-1) gene in ovarian tissue as a diagnosis subject, (b) a storage device configured to store a normal value of the expression amount of the ARL-1 gene in normal ovarian tissue, and (c) a determination mechanism configured to determine that the ovarian tissue as the diagnosis subject is mucinous adenocarcinoma when the detected value is higher than the normal value.

A fifth aspect of the present invention inheres in a molecular diagnosis system of ovarian cancers encompassing (a) a detection device configured to obtain a detected value of an expression amount of a bone marrow stromal cell antigen 2 (BST2) gene in ovarian tissue as a diagnosis subject (b) a storage device configured to store a normal value of the expression amount of the BST2 gene in normal ovarian tissue, and (c) a determination mechanism configured to determine that the ovarian tissue as the diagnosis subject is epithelial ovarian carcinoma when the detected value is lower than the normal value.

A sixth aspect of the present invention inheres in a molecular diagnosis system of ovarian cancers encompassing (a) a detection device configured to obtain a detected value of an expression amount of a cyclin E1 (CCNE1) gene in ovarian tissue as a diagnosis subject, (b) a storage device configured to store a normal value of the expression amount of the CCNE1 gene in normal ovarian tissue, and (c) a determination mechanism configured to determine that the ovarian tissue as the diagnosis subject is clear cell adenocarcinoma or serous adenocarcinoma when the detected value is higher than the normal value.

A seventh aspect of the present invention inheres in a molecular diagnosis system of ovarian cancers encompassing (a) a detection device configured to obtain a detected value of an expression amount of a cyclin-dependent kinase 4 (CDK4) gene in ovarian tissue as a diagnosis subject, (b) a storage device configured to store a normal value of the expression amount of the CDK4 gene in normal ovarian tissue, and (c) a determination mechanism configured to determine that the ovarian tissue as the diagnosis subject is epithelial ovarian carcinoma when the detected value is lower than the normal value.

An eighth aspect of the present invention inheres in a molecular diagnosis system of ovarian cancers encompassing (a) a detection device configured to obtain a detected value of an expression amount of a catenin, beta-1 (CTNNB1) gene in ovarian tissue as a diagnosis subject, (b) a storage device configured to store a normal value of the expression amount of the CTNNB1 gene in normal ovarian tissue, and (c) a determination mechanism configured to determine that the ovarian tissue as the diagnosis subject is epithelial ovarian carcinoma when the detected value is lower than the normal value.

A ninth aspect of the present invention inheres in a molecular diagnosis system of ovarian cancers encompassing (a) a detection device configured to obtain a detected value of an expression amount of a V-erb-b2 erythroblastic leukemia viral oncogene homolog 2 (ERBB2) gene in ovarian tissue as a diagnosis subject, (b) a storage device configured to store a normal value of the expression amount of the ERBB2 gene in normal ovarian tissue, and (c) a determination mechanism configured to determine that the ovarian tissue as the diagnosis subject is clear cell adenocarcinoma or serous adenocarcinoma when the detected value is higher than the normal value.

A tenth aspect of the present invention inheres in a molecular diagnosis system of ovarian cancers encompassing (a) a detection device configured to obtain a detected value of an expression amount of an estrogen receptor 1 (ESR1) gene in ovarian tissue as a diagnosis subject, (b) a storage device configured to store a normal value of the expression amount of the ESR1 gene in normal ovarian tissue, and (c) a determination mechanism configured to determine that the ovarian tissue as the diagnosis subject is mucinous adenocarcinoma or clear cell adenocarcinoma when the detected value is lower than the normal value.

An eleventh aspect of the present invention inheres in a molecular diagnosis system of ovarian cancers encompassing (a) a detection device configured to obtain a detected value of an expression amount of a human ovarian cancer specific transcript 2 (HOST2) gene in ovarian tissue as a diagnosis subject, (b) a storage device configured to store a normal value of the expression amount of the HOST2 gene in normal ovarian tissue, and (c) a determination mechanism configured to determine that the ovarian tissue as the diagnosis subject is clear cell adenocarcinoma, endometrioid adenocarcinoma or serous adenocarcinoma when the detected value is higher than the normal value.

A twelfth aspect of the present invention inheres in a molecular diagnosis system of ovarian cancers encompassing (a) a detection device configured to obtain a detected value of an expression amount of a hydroxysteroid (17-beta) dehydrogenase 1 (HSD17B1) gene in ovarian tissue as a diagnosis subject, (b) a storage device configured to store a normal value of the expression amount of the HSD17B1 gene in normal ovarian tissue, and (c) a determination mechanism configured to determine that the ovarian tissue as the diagnosis subject is clear cell adenocarcinoma or serous adenocarcinoma when the detected value is lower than the normal value.

A thirteenth aspect of the present invention inheres in a molecular diagnosis system of ovarian cancers encompassing (a) a detection device configured to obtain a detected value of an expression amount of an insulin-like growth factor binding protein 4 (IGFBP4) gene in ovarian tissue as a diagnosis subject, (b) a storage device configured to store a normal value of the expression amount of the IGFBP4 gene in normal ovarian tissue, and (c) a determination mechanism configured to determine that the ovarian tissue as the diagnosis subject is epithelial ovarian carcinoma when the detected value is lower than the normal value.

A fourteenth aspect of the present invention inheres in a molecular diagnosis system of ovarian cancers encompassing (a) a detection device configured to obtain a detected value of an expression amount of an insulin-like growth factor binding protein 6 (IGFBP6) gene in ovarian tissue as a diagnosis subject, (b) a storage device configured to store a normal value of the expression amount of the IGFBP6 gene in normal ovarian tissue, and (c) a determination mechanism configured to determine that the ovarian tissue as the diagnosis subject is epithelial ovarian carcinoma when the detected value is lower than the normal value.

A fifteenth aspect of the present invention inheres in a molecular diagnosis system of ovarian cancers encompassing (a) a detection device configured to obtain a detected value of an expression amount of an inhibin alpha (INHA) gene in ovarian tissue as a diagnosis subject, (b) a storage device configured to store a normal value of the expression amount of the INHA gene in normal ovarian tissue, and (c) a determination mechanism configured to determine that the ovarian tissue as the diagnosis subject is epithelial ovarian carcinoma when the detected value is lower than the normal value.

A sixteenth aspect of the present invention inheres in a molecular diagnosis system of ovarian cancers encompassing (a) a detection device configured to obtain a detected value of an expression amount of a keratin 7 (KRT7) gene in ovarian tissue as a diagnosis subject, (b) a storage device configured to store a normal value of the expression amount of the KRT7 gene in normal ovarian tissue, and (c) a determination mechanism configured to determine that the ovarian tissue as the diagnosis subject is epithelial ovarian carcinoma when the detected value is higher than the normal value.

A seventeenth aspect of the present invention inheres in a molecular diagnosis system of ovarian cancers encompassing (a) a detection device configured to obtain a detected value of an expression amount of a laminin, alpha 2 (LAMA2) gene in ovarian tissue as a diagnosis subject, (b) a storage device configured to store a normal value of the expression amount of the LAMA2 gene in normal ovarian tissue, and (c) a determination mechanism configured to determine that the ovarian tissue as the diagnosis subject is epithelial ovarian carcinoma when the detected value is lower than the normal value.

An eighteenth aspect of the present invention inheres in a molecular diagnosis system of ovarian cancers encompassing (a) a detection device configured to obtain a detected value of an expression amount of a matrix metallopeptidase 2 (MMP2) gene in ovarian tissue as a diagnosis subject, (b) a storage device configured to store a normal value of the expression amount of the MMP2 gene in normal ovarian tissue, and (c) a determination mechanism configured to determine that the ovarian tissue as the diagnosis subject is mucinous adenocarcinoma, clear cell adenocarcinoma or serous adenocarcinoma when the detected value is lower than the normal value.

A nineteenth aspect of the present invention inheres in a molecular diagnosis system of ovarian cancers encompassing (a) a detection device configured to obtain a detected value of an expression amount of a tissue inhibitor of metalloproteinase 1 (TIMP1) gene in ovarian tissue as a diagnosis subject, (b) a storage device configured to store a normal value of the expression amount of the TIMP1 gene in normal ovarian tissue, and (c) a determination mechanism configured to determine that the ovarian tissue as the diagnosis subject is epithelial ovarian carcinoma when the detected value is lower than the normal value.

A twentieth aspect of the present invention inheres in a molecular diagnosis method of ovarian cancers encompassing (a) obtaining a detected value of an expression amount of an ApoA1 gene in ovarian tissue as a diagnosis subject, and (b) determining that the ovarian tissue as the diagnosis subject is clear cell adenocarcinoma when the detected value is lower than a normal value of the expression amount of the ApoA1 gene in normal ovarian tissue.

A twenty-first aspect of the present invention inheres in a molecular diagnosis method of ovarian cancers encompassing (a) obtaining a detected value of an expression amount of an ApoE gene in ovarian tissue as a diagnosis subject, and (b) determining that the ovarian tissue as the diagnosis subject is epithelial ovarian carcinoma when the detected value is lower than a normal value of the expression amount of the ApoE gene in normal ovarian tissue.

A twenty-second aspect of the present invention inheres in a molecular diagnosis method of ovarian cancers encompassing (a) obtaining a detected value of an expression amount of an ApoJ gene in ovarian tissue as a diagnosis subject, and (b) determining that the ovarian tissue as the diagnosis subject is mucinous adenocarcinoma or clear cell adenocarcinoma when the detected value is lower than a normal value of the expression amount of the ApoJ gene in normal ovarian tissue.

A twenty-third aspect of the present invention inheres in a molecular diagnosis method of ovarian cancers encompassing (a) obtaining a detected value of an expression amount of an ARL-1 gene in ovarian tissue as a diagnosis subject, and (b) determining that the ovarian tissue as the diagnosis subject is mucinous adenocarcinoma when the detected value is higher than a normal value of the expression amount of the ARL-1 gene in normal ovarian tissue.

A twenty-fourth aspect of the present invention inheres in a molecular diagnosis method of ovarian cancers encompassing (a) obtaining a detected value of an expression amount of a BST2 gene in ovarian tissue as a diagnosis subject, and (b) determining that the ovarian tissue as the diagnosis subject is epithelial ovarian carcinoma when the detected value is lower than a normal value of the expression amount of the BST2 gene in normal ovarian tissue.

A twenty-fifth aspect of the present invention inheres in a molecular diagnosis method of ovarian cancers encompassing (a) obtaining a detected value of an expression amount of a CCNE1 gene in ovarian tissue as a diagnosis subject, and (b) determining that the ovarian tissue as the diagnosis subject is clear cell adenocarcinoma or serous adenocarcinoma when the detected value is higher than a normal value of the expression amount of the CCNE1 gene in normal ovarian tissue.

A twenty-sixth aspect of the present invention inheres in a molecular diagnosis method of ovarian cancers encompassing (a) obtaining a detected value of an expression amount of a CDK4 gene in ovarian tissue as a diagnosis subject, and (b) determining that the ovarian tissue as the diagnosis subject is epithelial ovarian carcinoma when the detected value is lower than a normal value of the expression amount of the CDK4 gene in normal ovarian tissue.

A twenty-seventh aspect of the present invention inheres in a molecular diagnosis method of ovarian cancers encompassing (a) obtaining a detected value of an expression amount of a CTNNB1 gene in ovarian tissue as a diagnosis subject, and (b) determining that the ovarian tissue as the diagnosis subject is epithelial ovarian carcinoma when the detected value is lower than a normal value of the expression amount of the CTNNB1 gene in normal ovarian tissue.

A twenty-eighth aspect of the present invention inheres in a molecular diagnosis method of ovarian cancers encompassing (a) obtaining a detected value of an expression amount of an ERBB2 gene in ovarian tissue as a diagnosis subject, and (b) determining that the ovarian tissue as the diagnosis subject is clear cell adenocarcinoma or serous adenocarcinoma when the detected value is higher than a normal value of the expression amount of the ERBB2 gene in normal ovarian tissue.

A twenty-ninth aspect of the present invention inheres in a molecular diagnosis method of ovarian cancers encompassing (a) obtaining a detected value of an expression amount of an ESR1 gene in ovarian tissue as a diagnosis subject, and (b) determining that the ovarian tissue as the diagnosis subject is mucinous adenocarcinoma or clear cell adenocarcinoma when the detected value is lower than a normal value of the expression amount of the ESR1 gene in normal ovarian tissue.

A thirtieth aspect of the present invention inheres in a molecular diagnosis method of ovarian cancers encompassing (a) obtaining a detected value of an expression amount of a HOST2 gene in ovarian tissue as a diagnosis subject, and (b) determining that the ovarian tissue as the diagnosis subject is clear cell adenocarcinoma, endometrioid adenocarcinoma or serous adenocarcinoma when the detected value is higher than a normal value of the expression amount of the HOST2 gene in normal ovarian tissue.

A thirty-first aspect of the present invention inheres in a molecular diagnosis method of ovarian cancers encompassing (a) obtaining a detected value of an expression amount of a HSD17B1 gene in ovarian tissue as a diagnosis subject, and (b) determining that the ovarian tissue as the diagnosis subject is clear cell adenocarcinoma or serous adenocarcinoma when the detected value is lower than a normal value of the expression amount of the HSD17B1 gene in normal ovarian tissue.

A thirty-second aspect of the present invention inheres in a molecular diagnosis method of ovarian cancers encompassing (a) obtaining a detected value of an expression amount of an IGFBP4 gene in ovarian tissue as a diagnosis subject, and (b) determining that the ovarian tissue as the diagnosis subject is epithelial ovarian carcinoma when the detected value is lower than a normal value of the expression amount of the IGFBP4 gene in normal ovarian tissue.

A thirty-third aspect of the present invention inheres in a molecular diagnosis method of ovarian cancers encompassing (a) obtaining a detected value of an expression amount of an IGFBP6 gene in ovarian tissue as a diagnosis subject, and (b) determining that the ovarian tissue as the diagnosis subject is epithelial ovarian carcinoma when the detected value is lower than a normal value of the expression amount of the IGFBP6 gene in normal ovarian tissue.

A thirty-fourth aspect of the present invention inheres in a molecular diagnosis method of ovarian cancers encompassing (a) obtaining a detected value of an expression amount of an INHA gene in ovarian tissue as a diagnosis subject, and (b) determining that the ovarian tissue as the diagnosis subject is epithelial ovarian carcinoma when the detected value is lower than a normal value of the expression amount of the INHA gene in normal ovarian tissue.

A thirty-fifth aspect of the present invention inheres in a molecular diagnosis method of ovarian cancers encompassing (a) obtaining a detected value of an expression amount of a KRT7 gene in ovarian tissue as a diagnosis subject, and (b) determining that the ovarian tissue as the diagnosis subject is epithelial ovarian carcinoma when the detected value is higher than a normal value of the expression amount of the KRT7 gene in normal ovarian tissue.

A thirty-sixth aspect of the present invention inheres in a molecular diagnosis method of ovarian cancers encompassing (a) obtaining a detected value of an expression amount of a LAMA2 gene in ovarian tissue as a diagnosis subject, and (b) determining that the ovarian tissue as the diagnosis subject is epithelial ovarian carcinoma when the detected value is lower than a normal value of the expression amount of the LAMA2 gene in normal ovarian tissue.

A thirty-seventh aspect of the present invention inheres in a molecular diagnosis method of ovarian cancers encompassing (a) obtaining a detected value of an expression amount of a MMP2 gene in ovarian tissue as a diagnosis subject, and (b) determining that the ovarian tissue as the diagnosis subject is mucinous adenocarcinoma, clear cell adenocarcinoma or serous adenocarcinoma when the detected value is lower than a normal value of the expression amount of the MMP2 gene in normal ovarian tissue.

A thirty-eighth aspect of the present invention inheres in a molecular diagnosis method of ovarian cancers encompassing (a) obtaining a detected value of an expression amount of a TIMP1 gene in ovarian tissue as a diagnosis subject, and (b) determining that the ovarian tissue as the diagnosis subject is epithelial ovarian carcinoma when the detected value is lower than a normal value of the expression amount of the TIMP1 gene in normal ovarian tissue.

### BRIEF DESCRIPTION OF THE DRAWINGS

FIG. 1 is a schematic diagram of a molecular diagnosis system of ovarian cancers according to a first embodiment of the present invention;

FIG. 2 is a first table showing detected intensities of anti-sense RNAs of an ApoA1_{—}1 gene according to an example of the present invention;

FIG. 3 is a second table showing detected intensities of anti-sense RNAs of an ApoA1_{—}1 gene according to an example of the present invention;

FIG. 4 is a third table showing detected intensities of anti-sense RNAs of an ApoA1_{—}1 gene according to an example of the present invention;

FIG. 5 is a fourth table showing detected intensities of anti-sense RNAs of an ApoA1_{—}1 gene according to an example of the present invention;

FIG. 6 is a fifth table showing detected intensities of anti-sense RNAs of an ApoA1_{—}1 gene according to an example of the present invention;

FIG. 7 is a sixth table showing detected intensities of anti-sense RNAs of an ApoA1_{—}1 gene according to an example of the present invention;

FIG. 8 is a first table showing detected intensities of anti-sense RNAs of an ApoE_{—}2 gene according to an example of the present invention;

FIG. 9 is a second table showing detected intensities of anti-sense RNAs of an ApoE_{—}2 gene according to an example of the present invention;

FIG. 10 is a third table showing detected intensities of anti-sense RNAs of an ApoE_{—}2 gene according to an example of the present invention;

FIG. 11 is a fourth table showing detected intensities of anti-sense RNAs of an ApoE_{—}2 gene according to an example of the present invention;

FIG. 12 is a fifth table showing detected intensities of anti-sense RNAs of an ApoE_{—}2 gene according to an example of the present invention;

FIG. 13 is a sixth table showing detected intensities of anti-sense RNAs of an ApoE_{—}2 gene according to an example of the present invention;

FIG. 14 is a first table showing detected intensities of anti-sense RNAs of an ApoJ_{—}1 gene according to an example of the present invention;

FIG. 15 is a second table showing detected intensities of anti-sense RNAs of an ApoJ_{—}1 gene according to an example of the present invention;

FIG. 16 is a third table showing detected intensities of anti-sense RNAs of an ApoJ_{—}1 gene according to an example of the present invention;

FIG. 17 is a fourth table showing detected intensities of anti-sense RNAs of an ApoJ_{—}1 gene according to an example of the present invention;

FIG. 18 is a fifth table showing detected intensities of anti-sense RNAs of an ApoJ_{—}1 gene according to an example of the present invention;

FIG. 19 is a sixth table showing detected intensities of anti-sense RNAs of an ApoJ_{—}1 gene according to an example of the present invention;

FIG. 20 is a first table showing detected intensities of anti-sense RNAs of an ARL-1_{—}1 gene according to an example of the present invention;

FIG. 21 is a second table showing detected intensities of anti-sense RNAs of an ARL-1_{—}1 gene according to an example of the present invention;

FIG. 22 is a third table showing detected intensities of anti-sense RNAs of an ARL-1_{—}1 gene according to an example of the present invention;

FIG. 23 is a fourth table showing detected intensities of anti-sense RNAs of an ARL-1_{—}1 gene according to an example of the present invention;

FIG. 24 is a fifth table showing detected intensities of anti-sense RNAs of an ARL-1_{—}1 gene according to an example of the present invention;

FIG. 25 is a sixth table showing detected intensities of anti-sense RNAs of an ARL-1_{—}1 gene according to an example of the present invention;

FIG. 26 is a first table showing detected intensities of anti-sense RNAs of a BST2_{—}1 gene according to an example of the present invention;

FIG. 27 is a second table showing detected intensities of anti-sense RNAs of a BST2_{—}1 gene according to an example of the present invention;

FIG. 28 is a third table showing detected intensities of anti-sense RNAs of a BST2_{—}1 gene according to an example of the present invention;

FIG. 29 is a fourth table showing detected intensities of anti-sense RNAs of a BST2_{—}1 gene according to an example of the present invention;

FIG. 30 is a fifth table showing detected intensities of anti-sense RNAs of a BST2_{—}1 gene according to an example of the present invention;

FIG. 31 is a sixth table showing detected intensities of anti-sense RNAs of a BST2_{—}1 gene according to an example of the present invention;

FIG. 32 is a first table showing detected intensities of anti-sense RNAs of a CCNE1_{—}1 gene according to an example of the present invention;

FIG. 33 is a second table showing detected intensities of anti-sense RNAs of a CCNE1_{—}1 gene according to an example of the present invention;

FIG. 34 is a third table showing detected intensities of anti-sense RNAs of a CCNE1_{—}1 gene according to an example of the present invention;

FIG. 35 is a fourth table showing detected intensities of anti-sense RNAs of a CCNE1_{—}1 gene according to an example of the present invention;

FIG. 36 is a fifth table showing detected intensities of anti-sense RNAs of a CCNE1_{—}1 gene according to an example of the present invention;

FIG. 37 is a sixth table showing detected intensities of anti-sense RNAs of a CCNE1_{—}1 gene according to an example of the present invention;

FIG. 38 is a first table showing detected intensities of anti-sense RNAs of a CDK4 gene according to an example of the present invention;

FIG. 39 is a second table showing detected intensities of anti-sense RNAs of a CDK4 gene according to an example of the present invention;

FIG. 40 is a third table showing detected intensities of anti-sense RNAs of a CDK4 gene according to an example of the present invention;

FIG. 41 is a fourth table showing detected intensities of anti-sense RNAs of a CDK4 gene according to an example of the present invention;

FIG. 42 is a fifth table showing detected intensities of anti-sense RNAs of a CDK4 gene according to an example of the present invention;

FIG. 43 is a sixth table showing detected intensities of anti-sense RNAs of a CDK4 gene according to an example of the present invention;

FIG. 44 is a first table showing detected intensities of anti-sense RNAs of a CTNNB1 gene according to an example of the present invention;

FIG. 45 is a second table showing detected intensities of anti-sense RNAs of a CTNNB1 gene according to an example of the present invention;

FIG. 46 is a third table showing detected intensities of anti-sense RNAs of a CTNNB1 gene according to an example of the present invention;

FIG. 47 is a fourth table showing detected intensities of anti-sense RNAs of a CTNNB1 gene according to an example of the present invention;

FIG. 48 is a fifth table showing detected intensities of anti-sense RNAs of a CTNNB1 gene according to an example of the present invention;

FIG. 49 is a sixth table showing detected intensities of anti-sense RNAs of a CTNNB1 gene according to an example of the present invention;

FIG. 50 is a first table showing detected intensities of anti-sense RNAs of an ERBB2_{—}1 gene according to an example of the present invention;

FIG. 51 is a second table showing detected intensities of anti-sense RNAs of an ERBB2_{—}1 gene according to an example of the present invention;

FIG. 52 is a third table showing detected intensities of anti-sense RNAs of an ERBB2_{—}1 gene according to an example of the present invention;

FIG. 53 is a fourth table showing detected intensities of anti-sense RNAs of an ERBB2_{—}1 gene according to an example of the present invention;

FIG. 54 is a fifth table showing detected intensities of anti-sense RNAs of an ERBB2_{—}1 gene according to an example of the present invention;

FIG. 55 is a sixth table showing detected intensities of anti-sense RNAs of an ERBB2_{—}1 gene according to an example of the present invention;

FIG. 56 is a first table showing detected intensities of anti-sense RNAs of an ESR1 gene according to an example of the present invention;

FIG. 57 is a second table showing detected intensities of anti-sense RNAs of an ESR1 gene according to an example of the present invention;

FIG. 58 is a third table showing detected intensities of anti-sense RNAs of an ESR1 gene according to an example of the present invention;

FIG. 59 is a fourth table showing detected intensities of anti-sense RNAs of an ESR1 gene according to an example of the present invention;

FIG. 60 is a fifth table showing detected intensities of anti-sense RNAs of an ESR1 gene according to an example of the present invention;

FIG. 61 is a sixth table showing detected intensities of anti-sense RNAs of an ESR1 gene according to an example of the present invention;

FIG. 62 is a first table showing detected intensities of anti-sense RNAs of a HOST2 gene according to an example of the present invention;

FIG. 63 is a second table showing detected intensities of anti-sense RNAs of a HOST2 gene according to an example of the present invention;

FIG. 64 is a third table showing detected intensities of anti-sense RNAs of a HOST2 gene according to an example of the present invention;

FIG. 65 is a fourth table showing detected intensities of anti-sense RNAs of a HOST2 gene according to an example of the present invention;

FIG. 66 is a fifth table showing detected intensities of anti-sense RNAs of a HOST2 gene according to an example of the present invention;

FIG. 67 is a sixth table showing detected intensities of anti-sense RNAs of a HOST2 gene according to an example of the present invention;

FIG. 68 is a first table showing detected intensities of anti-sense RNAs of a HSD17B1 gene according to an example of the present invention;

FIG. 69 is a second table showing detected intensities of anti-sense RNAs of a HSD17B1 gene according to an example of the present invention;

FIG. 70 is a third table showing detected intensities of anti-sense RNAs of a HSD17B1 gene according to an example of the present invention;

FIG. 71 is a fourth table showing detected intensities of anti-sense RNAs of a HSD17B1 gene according to an example of the present invention;

FIG. 72 is a fifth table showing detected intensities of anti-sense RNAs of a HSD17B1 gene according to an example of the present invention;

FIG. 73 is a sixth table showing detected intensities of anti-sense RNAs of a HSD17B1 gene according to an example of the present invention;

FIG. 74 is a first table showing detected intensities of anti-sense RNAs of an IGFBP4 gene according to an example of the present invention;

FIG. 75 is a second table showing detected intensities of anti-sense RNAs of an IGFBP4 gene according to an example of the present invention;

FIG. 76 is a third table showing detected intensities of anti-sense RNAs of an IGFBP4 gene according to an example of the present invention;

FIG. 77 is a fourth table showing detected intensities of anti-sense RNAs of an IGFBP4 gene according to an example of the present invention;

FIG. 78 is a fifth table showing detected intensities of anti-sense RNAs of an IGFBP4 gene according to an example of the present invention;

FIG. 79 is a sixth table showing detected intensities of anti-sense RNAs of an IGFBP4 gene according to an example of the present invention;

FIG. 80 is a first table showing detected intensities of anti-sense RNAs of an IGFBP6 gene according to an example of the present invention;

FIG. 81 is a second table showing detected intensities of anti-sense RNAs of an IGFBP6 gene according to an example of the present invention;

FIG. 82 is a third table showing detected intensities of anti-sense RNAs of an IGFBP6 gene according to an example of the present invention;

FIG. 83 is a fourth table showing detected intensities of anti-sense RNAs of an IGFBP6 gene according to an example of the present invention;

FIG. 84 is a fifth table showing detected intensities of anti-sense RNAs of an IGFBP6 gene according to an example of the present invention;

FIG. 85 is a sixth table showing detected intensities of anti-sense RNAs of an IGFBP6 gene according to an example of the present invention;

FIG. 86 is a first table showing detected intensities of anti-sense RNAs of an INHA gene according to an example of the present invention;

FIG. 87 is a second table showing detected intensities of anti-sense RNAs of an INHA gene according to an example of the present invention;

FIG. 88 is a third table showing detected intensities of anti-sense RNAs of an INHA gene according to an example of the present invention;

FIG. 89 is a fourth table showing detected intensities of anti-sense RNAs of an INHA gene according to an example of the present invention;

FIG. 90 is a fifth table showing detected intensities of anti-sense RNAs of an INHA gene according to an example of the present invention;

FIG. 91 is a sixth table showing detected intensities of anti-sense RNAs of an INHA gene according to an example of the present invention;

FIG. 92 is a first table showing detected intensities of anti-sense RNAs of a KRT7_{—}1 gene according to an example of the present invention;

FIG. 93 is a second table showing detected intensities of anti-sense RNAs of a KRT7_{—}1 gene according to an example of the present invention;

FIG. 94 is a third table showing detected intensities of anti-sense RNAs of a KRT7_{—}1 gene according to an example of the present invention;

FIG. 95 is a fourth table showing detected intensities of anti-sense RNAs of a KRT7_{—}1 gene according to an example of the present invention;

FIG. 96 is a fifth table showing detected intensities of anti-sense RNAs of a KRT7_{—}1 gene according to an example of the present invention;

FIG. 97 is a sixth table showing detected intensities of anti-sense RNAs of a KRT7_{—}1 gene according to an example of the present invention;

FIG. 98 is a first table showing detected intensities of anti-sense RNAs of a LAMA2 gene according to an example of the present invention;

FIG. 99 is a second table showing detected intensities of anti-sense RNAs of a LAMA2 gene according to an example of the present invention;

FIG. 100 is a third table showing detected intensities of anti-sense RNAs of a LAMA2 gene according to an example of the present invention;

FIG. 101 is a fourth table showing detected intensities of anti-sense RNAs of a LAMA2 gene according to an example of the present invention;

FIG. 102 is a fifth table showing detected intensities of anti-sense RNAs of a LAMA2 gene according to an example of the present invention;

FIG. 103 is a sixth table showing detected intensities of anti-sense RNAs of a LAMA2 gene according to an example of the present invention;

FIG. 104 is a first table showing detected intensities of anti-sense RNAs of a MMP2 gene according to an example of the present invention;

FIG. 105 is a second table showing detected intensities of anti-sense RNAs of a MMP2 gene according to an example of the present invention;

FIG. 106 is a third table showing detected intensities of anti-sense RNAs of a MMP2 gene according to an example of the present invention;

FIG. 107 is a fourth table showing detected intensities of anti-sense RNAs of a MMP2 gene according to an example of the present invention;

FIG. 108 is a fifth table showing detected intensities of anti-sense RNAs of a MMP2 gene according to an example of the present invention;

FIG. 109 is a sixth table showing detected intensities of anti-sense RNAs of a MMP2 gene according to an example of the present invention;

FIG. 110 is a first table showing detected intensities of anti-sense RNAs of a TIMP1_{—}1 gene according to an example of the present invention;

FIG. 111 is a second table showing detected intensities of anti-sense RNAs of a TIMP1_{—}1 gene according to an example of the present invention;

FIG. 112 is a third table showing detected intensities of anti-sense RNAs of a TIMP1_{—}1 gene according to an example of the present invention;

FIG. 113 is a fourth table showing detected intensities of anti-sense RNAs of a TIMP1_{—}1 gene according to an example of the present invention;

FIG. 114 is a fifth table showing detected intensities of anti-sense RNAs of a TIMP1_{—}1 gene according to an example of the present invention; and

FIG. 115 is a sixth table showing detected intensities of anti-sense RNAs of a TIMP1_{—}1 gene according to an example of the present invention.

### DETAILED DESCRIPTION OF THE INVENTION

A description will be made below of an embodiment of the present invention. In the following description made with reference to the drawings, the same or similar portions are denoted by the same or similar reference numerals. Note that the drawings are schematic. Hence, specific dimensions and the like should be determined with reference to the following description. Moreover, it is a matter of course that portions different in dimensional relationship and ratio from one another is also included in the drawings.

### First Embodiment

As shown in FIG. 1, a molecular diagnosis system of ovarian cancers according to a first embodiment encompasses a detection device 100 configured to obtain a detected value of an expression amount of an ApoA1 gene in ovarian tissue as a diagnosis subject, and a central processing unit (CPU) 300 connected to the detection device 100. To the CPU 300, a storage device 200 is connected, which stores a normal value of the expression amount of the ApoA1 gene in normal ovarian tissue. Moreover, the CPU 300 includes a determination mechanism 301 configured to determine that the ovarian tissue as the diagnosis subject is clear cell adenocarcinoma when the detected value of the expression amount of the ApoA1 gene, which is obtained by the detection device 100, is lower than the normal value stored in the storage device 200. Note that the expression amount of the ApoA1 gene includes not only the expression amount of ApoA1 itself but also an expression amount of a gene (DNAs or RNAs) that codes the ApoA1.

The detection device 100 is, for example, a scanner for a nucleotide chip. In the nucleotide chip, polynucleotide complementary to a part or entirety of the gene that codes the ApoA1 is fixed as a probe to a substrate. When genes extracted from the ovarian tissue as the diagnosis subject are dropped onto the nucleotide chip, only the gene that codes the ApoA1 is captured by the nucleotide chip. The detection device 100 obtains an amount of such an ApoA1-coding gene, which is captured by the nucleotide chip, as the detected value of the expression amount of the ApoA1 gene.

Moreover, a detected value by the detection device 100, which is an amount of a gene extracted from the ovarian tissue previously diagnosed not to be the cancer but to be normal, thus derived from the normal ovarian tissue, and captured by the nucleotide chip by being dropped thereon, is stored as the normal value in the storage device 200.

The determination mechanism 301 of the CPU 300 receives, from the detection device 100, the detected value of the expression amount of the ApoA1 gene derived from the ovarian tissue as the diagnosis subject, and reads out, from the storage device 200, the normal value of the expression amount of the ApoA1 gene derived from the normal ovarian tissue. Moreover, the determination mechanism 301 compares the detected value of the expression amount of the ApoA1 gene and the normal value of the expression amount of the ApoA1 gene with each other, and determines that the ovarian tissue as the diagnosis subject is the clear cell adenocarcinoma when the detected value is lower than the normal value. Reasons why it is determined that the ovarian tissue as the diagnosis subject is the clear cell adenocarcinoma when the detected value is lower than the normal value will be described later.

### Second Embodiment

In a molecular diagnosis system of ovarian cancers according to a second embodiment, the detection device 100 shown in FIG. 1 obtains a detected value of an expression amount of an ApoE gene in the ovarian tissue as the diagnosis subject, and the storage device 200 stores a normal value of the expression amount of the ApoE gene in the normal ovarian tissue. Moreover, the determination mechanism 301 determines that the ovarian tissue as the diagnosis subject is epithelial ovarian carcinoma when the detected value of the expression amount of the ApoE gene, which is obtained by the detection device 100, is lower than the normal value stored in the storage device 200. Note that the expression amount of the ApoE gene includes not only the expression amount of ApoE itself but also an expression amount of a gene (DNAs or RNAs) that codes the ApoE.

In the nucleotide chip disposed in the detection device 100, polynucleotide complementary to a part or entirety of the gene that codes the ApoE is fixed as a probe to a substrate. When genes extracted from the ovarian tissue as the diagnosis subject are dropped onto the nucleotide chip, only the gene that codes the ApoE is captured by the nucleotide chip. The detection device 100 obtains an amount of such an ApoE-coding gene, which is captured by the nucleotide chip, as the detected value of the expression amount of the ApoE gene.

The determination mechanism 301 of the CPU 300 receives, from the detection device 100, the detected value of the expression amount of the ApoE gene derived from the ovarian tissue as the diagnosis subject, and reads out, from the storage device 200, the normal value of the expression amount of the ApoE gene derived from the normal ovarian tissue. Moreover, the determination mechanism 301 compares the detected value of the expression amount of the ApoE gene and the normal value of the expression amount of the ApoE gene with each other, and determines that the ovarian tissue as the diagnosis subject is the epithelial ovarian carcinoma when the detected value is lower than the normal value. Reasons why it is determined that the ovarian tissue as the diagnosis subject is the epithelial ovarian carcinoma when the detected value is lower than the normal value will be described later.

### Third Embodiment

In a molecular diagnosis system of ovarian cancers according to a third embodiment, the detection device 100 shown in FIG. 1 obtains a detected value of an expression amount of an ApoJ gene in the ovarian tissue as the diagnosis subject, and the storage device 200 stores a normal value of the expression amount of the ApoJ gene in the normal ovarian tissue. Moreover, the determination mechanism 301 determines that the ovarian tissue as the diagnosis subject is mucinous adenocarcinoma or clear cell adenocarcinoma when the detected value of the expression amount of the ApoJ gene, which is obtained by the detection device 100, is lower than the normal value stored in the storage device 200. Note that the expression amount of the ApoJ gene includes not only the expression amount of ApoJ itself but also an expression amount of a gene (DNAs or RNAs) that codes the ApoJ.

In the nucleotide chip disposed in the detection device 100, polynucleotide complementary to a part or entirety of the gene that codes the ApoJ is fixed as a probe to a substrate. When genes extracted from the ovarian tissue as the diagnosis subject are dropped onto the nucleotide chip, only the gene that codes the ApoJ is captured by the nucleotide chip. The detection device 100 obtains an amount of such an ApoJ-coding gene, which is captured by the nucleotide chip, as the detected value of the expression amount of the ApoJ gene.

The determination mechanism 301 of the CPU 300 receives, from the detection device 100, the detected value of the expression amount of the ApoJ gene derived from the ovarian tissue as the diagnosis subject, and reads out, from the storage device 200, the normal value of the expression amount of the ApoJ gene derived from the normal ovarian tissue. Moreover, the determination mechanism 301 compares the detected value of the expression amount of the ApoJ gene and the normal value of the expression amount of the ApoJ gene with each other, and determines that the ovarian tissue as the diagnosis subject is the mucinous adenocarcinoma or the clear cell adenocarcinoma when the detected value is lower than the normal value. Reasons why it is determined that the ovarian tissue as the diagnosis subject is the mucinous adenocarcinoma or the clear cell adenocarcinoma when the detected value is lower than the normal value will be described later.

### Fourth Embodiment

In a molecular diagnosis system of ovarian cancers according to a fourth embodiment, the detection device 100 shown in FIG. 1 obtains a detected value of an expression amount of an ARL-1 gene in the ovarian tissue as the diagnosis subject, and the storage device 200 stores a normal value of the expression amount of the ARL-1 gene in the normal ovarian tissue. Moreover, the determination mechanism 301 determines that the ovarian tissue as the diagnosis subject is mucinous adenocarcinoma when the detected value of the expression amount of the ARL-1 gene, which is obtained by the detection device 100, is higher than the normal value stored in the storage device 200. Note that the expression amount of the ARL-1 gene includes not only the expression amount of ARL-1 itself but also an expression amount of a gene (DNAs or RNAs) that codes the ARL-1.

In the nucleotide chip disposed in the detection device 100, polynucleotide complementary to a part or entirety of the gene that codes the ARL-1 is fixed as a probe to a substrate. When genes extracted from the ovarian tissue as the diagnosis subject are dropped onto the nucleotide chip, only the gene that codes the ARL-1 is captured by the nucleotide chip. The detection device 100 obtains an amount of such an ARL-1-coding gene, which is captured by the nucleotide chip, as the detected value of the expression amount of the ARL-1 gene.

The determination mechanism 301 of the CPU 300 receives, from the detection device 100, the detected value of the expression amount of the ARL-1 gene derived from the ovarian tissue as the diagnosis subject, and reads out, from the storage device 200, the normal value of the expression amount of the ARL-1 gene derived from the normal ovarian tissue. Moreover, the determination mechanism 301 compares the detected value of the expression amount of the ARL-1 gene and the normal value of the expression amount of the ARL-1 gene with each other, and determines that the ovarian tissue as the diagnosis subject is the mucinous adenocarcinoma when the detected value is higher than the normal value. Reasons why it is determined that the ovarian tissue as the diagnosis subject is the mucinous adenocarcinoma when the detected value is higher than the normal value will be described later.

### Fifth Embodiment

In a molecular diagnosis system of ovarian cancers according to a fifth embodiment, the detection device 100 shown in FIG. 1 obtains a detected value of an expression amount of a BST2 gene in the ovarian tissue as the diagnosis subject, and the storage device 200 stores a normal value of the expression amount of the BST2 gene in the normal ovarian tissue. Moreover, the determination mechanism 301 determines that the ovarian tissue as the diagnosis subject is epithelial ovarian carcinoma when the detected value of the expression amount of the BST2 gene, which is obtained by the detection device 100, is lower than the normal value stored in the storage device 200. Note that the expression amount of the BST2 gene includes not only the expression amount of BST2 itself but also an expression amount of a gene (DNAs or RNAs) that codes the BST2.

In the nucleotide chip disposed in the detection device 100, polynucleotide complementary to a part or entirety of the gene that codes the BST2 is fixed as a probe to a substrate. When genes extracted from the ovarian tissue as the diagnosis subject are dropped onto the nucleotide chip, only the gene that codes the BST2 is captured by the nucleotide chip. The detection device 100 obtains an amount of such a BST2-coding gene, which is captured by the nucleotide chip, as the detected value of the expression amount of the BST2 gene.

The determination mechanism 301 of the CPU 300 receives, from the detection device 100, the detected value of the expression amount of the BST2 gene derived from the ovarian tissue as the diagnosis subject, and reads out, from the storage device 200, the normal value of the expression amount of the BST2 gene derived from the normal ovarian tissue. Moreover, the determination mechanism 301 compares the detected value of the expression amount of the BST2 gene and the normal value of the expression amount of the BST2 gene with each other, and determines that the ovarian tissue as the diagnosis subject is the epithelial ovarian carcinoma when the detected value is lower than the normal value. Reasons why it is determined that the ovarian tissue as the diagnosis subject is the epithelial ovarian carcinoma when the detected value is lower than the normal value will be described later.

### Sixth Embodiment

In a molecular diagnosis system of ovarian cancers according to a sixth embodiment, the detection device 100 shown in FIG. 1 obtains a detected value of an expression amount of a CCNE1 gene in the ovarian tissue as the diagnosis subject, and the storage device 200 stores a normal value of the expression amount of the CCNE1 gene in the normal ovarian tissue. Moreover, the determination mechanism 301 determines that the ovarian tissue as the diagnosis subject is clear cell adenocarcinoma or serous adenocarcinoma when the detected value of the expression amount of the CCNE1 gene, which is obtained by the detection device 100, is higher than the normal value stored in the storage device 200. Note that the expression amount of the CCNE1 gene includes not only the expression amount of CCNE1 itself but also an expression amount of a gene (DNAs or RNAs) that codes the CCNE1.

In the nucleotide chip disposed in the detection device 100, polynucleotide complementary to a part or entirety of the gene that codes the CCNE1 is fixed as a probe to a substrate. When genes extracted from the ovarian tissue as the diagnosis subject are dropped onto the nucleotide chip, only the gene that codes the CCNE1 is captured by the nucleotide chip. The detection device 100 obtains an amount of such a CCNE1-coding gene, which is captured by the nucleotide chip, as the detected value of the expression amount of the CCNE1 gene.

The determination mechanism 301 of the CPU 300 receives, from the detection device 100, the detected value of the expression amount of the CCNE1 gene derived from the ovarian tissue as the diagnosis subject, and reads out, from the storage device 200, the normal value of the expression amount of the CCNE1 gene derived from the normal ovarian tissue. Moreover, the determination mechanism 301 compares the detected value of the expression amount of the CCNE1 gene and the normal value of the expression amount of the CCNE1 gene with each other, and determines that the ovarian tissue as the diagnosis subject is the clear cell adenocarcinoma or the serous adenocarcinoma when the detected value is higher than the normal value. Reasons why it is determined that the ovarian tissue as the diagnosis subject is the clear cell adenocarcinoma or the serous adenocarcinoma when the detected value is higher than the normal value will be described later.

### Seventh Embodiment

In a molecular diagnosis system of ovarian cancers according to a seventh embodiment, the detection device 100 shown in FIG. 1 obtains a detected value of an expression amount of a CDK4 gene in the ovarian tissue as the diagnosis subject, and the storage device 200 stores a normal value of the expression amount of the CDK4 gene in the normal ovarian tissue. Moreover, the determination mechanism 301 determines that the ovarian tissue as the diagnosis subject is epithelial ovarian carcinoma when the detected value of the expression amount of the CDK4 gene, which is obtained by the detection device 100, is lower than the normal value stored in the storage device 200. Note that the expression amount of the CDK4 gene includes not only the expression amount of CDK4 itself but also an expression amount of a gene (DNAs or RNAs) that codes the CDK4.

In the nucleotide chip disposed in the detection device 100, polynucleotide complementary to a part or entirety of the gene that codes the CDK4 is fixed as a probe to a substrate. When genes extracted from the ovarian tissue as the diagnosis subject are dropped onto the nucleotide chip, only the gene that codes the CDK4 is captured by the nucleotide chip. The detection device 100 obtains an amount of such a CDK4-coding gene, which is captured by the nucleotide chip, as the detected value of the expression amount of the CDK4 gene.

The determination mechanism 301 of the CPU 300 receives, from the detection device 100, the detected value of the expression amount of the CDK4 gene derived from the ovarian tissue as the diagnosis subject, and reads out, from the storage device 200, the normal value of the expression amount of the CDK4 gene derived from the normal ovarian tissue. Moreover, the determination mechanism 301 compares the detected value of the expression amount of the CDK4 gene and the normal value of the expression amount of the CDK4 gene with each other, and determines that the ovarian tissue as the diagnosis subject is the epithelial ovarian carcinoma when the detected value is lower than the normal value. Reasons why it is determined that the ovarian tissue as the diagnosis subject is the epithelial ovarian carcinoma when the detected value is lower than the normal value will be described later.

### Eighth Embodiment

In a molecular diagnosis system of ovarian cancers according to a eighth embodiment, the detection device 100 shown in FIG. 1 obtains a detected value of an expression amount of a CTNNB1 gene in the ovarian tissue as the diagnosis subject, and the storage device 200 stores a normal value of the expression amount of the CTNNB1 gene in the normal ovarian tissue. Moreover, the determination mechanism 301 determines that the ovarian tissue as the diagnosis subject is epithelial ovarian carcinoma when the detected value of the expression amount of the CTNNB1 gene, which is obtained by the detection device 100, is lower than the normal value stored in the storage device 200. Note that the expression amount of the CTNNB1 gene includes not only the expression amount of CTNNB1 itself but also an expression amount of a gene (DNAs or RNAs) that codes the CTNNB1.

In the nucleotide chip disposed in the detection device 100, polynucleotide complementary to a part or entirety of the gene that codes the CTNNB1 is fixed as a probe to a substrate. When genes extracted from the ovarian tissue as the diagnosis subject are dropped onto the nucleotide chip, only the gene that codes the CTNNB1 is captured by the nucleotide chip. The detection device 100 obtains an amount of such a CTNNB1-coding gene, which is captured by the nucleotide chip, as the detected value of the expression amount of the CTNNB1 gene.

The determination mechanism 301 of the CPU 300 receives, from the detection device 100, the detected value of the expression amount of the CTNNB1 gene derived from the ovarian tissue as the diagnosis subject, and reads out, from the storage device 200, the normal value of the expression amount of the CTNNB1 gene derived from the normal ovarian tissue. Moreover, the determination mechanism 301 compares the detected value of the expression amount of the CTNNB1 gene and the normal value of the expression amount of the CTNNB1 gene with each other, and determines that the ovarian tissue as the diagnosis subject is the epithelial ovarian carcinoma when the detected value is lower than the normal value. Reasons why it is determined that the ovarian tissue as the diagnosis subject is the epithelial ovarian carcinoma when the detected value is lower than the normal value will be described later.

### Ninth Embodiment

In a molecular diagnosis system of ovarian cancers according to a ninth embodiment, the detection device 100 shown in FIG. 1 obtains a detected value of an expression amount of an ERBB2 gene in the ovarian tissue as the diagnosis subject, and the storage device 200 stores a normal value of the expression amount of the ERBB2 gene in the normal ovarian tissue. Moreover, the determination mechanism 301 determines that the ovarian tissue as the diagnosis subject is clear cell adenocarcinoma or serous adenocarcinoma when the detected value of the expression amount of the ERBB2 gene, which is obtained by the detection device 100, is higher than the normal value stored in the storage device 200. Note that the expression amount of the ERBB2 gene includes not only the expression amount of ERBB2 itself but also an expression amount of a gene (DNAs or RNAs) that codes the ERBB2.

In the nucleotide chip disposed in the detection device 100, polynucleotide complementary to a part or entirety of the gene that codes the ERBB2 is fixed as a probe to a substrate. When genes extracted from the ovarian tissue as the diagnosis subject are dropped onto the nucleotide chip, only the gene that codes the ERBB2 is captured by the nucleotide chip. The detection device 100 obtains an amount of such an ERBB2-coding gene, which is captured by the nucleotide chip, as the detected value of the expression amount of the ERBB2 gene.

The determination mechanism 301 of the CPU 300 receives, from the detection device 100, the detected value of the expression amount of the ERBB2 gene derived from the ovarian tissue as the diagnosis subject, and reads out, from the storage device 200, the normal value of the expression amount of the ERBB2 gene derived from the normal ovarian tissue. Moreover, the determination mechanism 301 compares the detected value of the expression amount of the ERBB2 gene and the normal value of the expression amount of the ERBB2 gene with each other, and determines that the ovarian tissue as the diagnosis subject is the clear cell adenocarcinoma or the serous adenocarcinoma when the detected value is higher than the normal value. Reasons why it is determined that the ovarian tissue as the diagnosis subject is the clear cell adenocarcinoma or the serous adenocarcinoma when the detected value is higher than the normal value will be described later.

### Tenth Embodiment

In a molecular diagnosis system of ovarian cancers according to a tenth embodiment, the detection device 100 shown in FIG. 1 obtains a detected value of an expression amount of an ESR1 gene in the ovarian tissue as the diagnosis subject, and the storage device 200 stores a normal value of the expression amount of the ESR1 gene in the normal ovarian tissue. Moreover, the determination mechanism 301 determines that the ovarian tissue as the diagnosis subject is mucinous adenocarcinoma or clear cell adenocarcinoma when the detected value of the expression amount of the ESR1 gene, which is obtained by the detection device 100, is lower than the normal value stored in the storage device 200.

In the nucleotide chip disposed in the detection device 100, polynucleotide complementary to a part or entirety of the gene that codes the ESR1 is fixed as a probe to a substrate. When genes extracted from the ovarian tissue as the diagnosis subject are dropped onto the nucleotide chip, only the gene that codes the ESR1 is captured by the nucleotide chip. The detection device 100 obtains an amount of such an ESR1-coding gene, which is captured by the nucleotide chip, as the detected value of the expression amount of the ESR1 gene. Note that the expression amount of the ESR1 gene includes not only the expression amount of ESR1 itself but also an expression amount of a gene (DNAs or RNAs) that codes the ESR1.

The determination mechanism 301 of the CPU 300 receives, from the detection device 100, the detected value of the expression amount of the ESR1 gene derived from the ovarian tissue as the diagnosis subject, and reads out, from the storage device 200, the normal value of the expression amount of the ESR1 gene derived from the normal ovarian tissue. Moreover, the determination mechanism 301 compares the detected value of the expression amount of the ESR1 gene and the normal value of the expression amount of the ESR1 gene with each other, and determines that the ovarian tissue as the diagnosis subject is the mucinous adenocarcinoma or the clear cell adenocarcinoma when the detected value is lower than the normal value. Reasons why it is determined that the ovarian tissue as the diagnosis subject is the mucinous adenocarcinoma or the clear cell adenocarcinoma when the detected value is lower than the normal value will be described later.

### Eleventh Embodiment

In a molecular diagnosis system of ovarian cancers according to a eleventh embodiment, the detection device 100 shown in FIG. 1 obtains a detected value of an expression amount of a HOST2 gene in the ovarian tissue as the diagnosis subject, and the storage device 200 stores a normal value of the expression amount of the HOST2 gene in the normal ovarian tissue. Moreover, the determination mechanism 301 determines that the ovarian tissue as the diagnosis subject is clear cell adenocarcinoma, endometrioid adenocarcinoma or serous adenocarcinoma when the detected value of the expression amount of the HOST2 gene, which is obtained by the detection device 100, is higher than the normal value stored in the storage device 200. Note that the expression amount of the HOST2 gene includes not only the expression amount of HOST2 itself but also an expression amount of a gene (DNAs or RNAs) that codes the HOST2.

In the nucleotide chip disposed in the detection device 100, polynucleotide complementary to a part or entirety of the gene that codes the HOST2 is fixed as a probe to a substrate. When genes extracted from the ovarian tissue as the diagnosis subject are dropped onto the nucleotide chip, only the gene that codes the HOST2 is captured by the nucleotide chip. The detection device 100 obtains an amount of such a HOST2-coding gene, which is captured by the nucleotide chip, as the detected value of the expression amount of the HOST2 gene.

The determination mechanism 301 of the CPU 300 receives, from the detection device 100, the detected value of the expression amount of the HOST2 gene derived from the ovarian tissue as the diagnosis subject, and reads out, from the storage device 200, the normal value of the expression amount of the HOST2 gene derived from the normal ovarian tissue. Moreover, the determination mechanism 301 compares the detected value of the expression amount of the HOST2 gene and the normal value of the expression amount of the HOST2 gene with each other, and determines that the ovarian tissue as the diagnosis subject is the clear cell adenocarcinoma, the endometrioid adenocarcinoma or the serous adenocarcinoma when the detected value is higher than the normal value. Reasons why it is determined that the ovarian tissue as the diagnosis subject is the clear cell adenocarcinoma, the endometrioid adenocarcinoma or the serous adenocarcinoma when the detected value is higher than the normal value will be described later.

### Twelfth Embodiment

In a molecular diagnosis system of ovarian cancers according to a twelfth embodiment, the detection device 100 shown in FIG. 1 obtains a detected value of an expression amount of a HSD17B1 gene in the ovarian tissue as the diagnosis subject, and the storage device 200 stores a normal value of the expression amount of the HSD17B1 gene in the normal ovarian tissue. Moreover, the determination mechanism 301 determines that the ovarian tissue as the diagnosis subject is clear cell adenocarcinoma or serous adenocarcinoma when the detected value of the expression amount of the HSD17B1 gene, which is obtained by the detection device 100, is lower than the normal value stored in the storage device 200. Note that the expression amount of the HSD17B1 gene includes not only the expression amount of HSD17B1 itself but also an expression amount of a gene (DNAs or RNAs) that codes the HSD17B1.

In the nucleotide chip disposed in the detection device 100, polynucleotide complementary to a part or entirety of the gene that codes the HSD17B1 is fixed as a probe to a substrate. When genes extracted from the ovarian tissue as the diagnosis subject are dropped onto the nucleotide chip, only the gene that codes the HSD17B1 is captured by the nucleotide chip. The detection device 100 obtains an amount of such a HSD17B1-coding gene, which is captured by the nucleotide chip, as the detected value of the expression amount of the HSD17B1 gene.

The determination mechanism 301 of the CPU 300 receives, from the detection device 100, the detected value of the expression amount of the HSD17B1 gene derived from the ovarian tissue as the diagnosis subject, and reads out, from the storage device 200, the normal value of the expression amount of the HSD17B1 gene derived from the normal ovarian tissue. Moreover, the determination mechanism 301 compares the detected value of the expression amount of the HSD17B1 gene and the normal value of the expression amount of the HSD17B1 gene with each other, and determines that the ovarian tissue as the diagnosis subject is the clear cell adenocarcinoma or the serous adenocarcinoma when the detected value is lower than the normal value. Reasons why it is determined that the ovarian tissue as the diagnosis subject is the clear cell adenocarcinoma or the serous adenocarcinoma when the detected value is lower than the normal value will be described later.

### Thirteenth Embodiment

In a molecular diagnosis system of ovarian cancers according to a thirteenth embodiment, the detection device 100 shown in FIG. 1 obtains a detected value of an expression amount of a IGFBP4 gene in the ovarian tissue as the diagnosis subject, and the storage device 200 stores a normal value of the expression amount of the IGFBP4 gene in the normal ovarian tissue. Moreover, the determination mechanism 301 determines that the ovarian tissue as the diagnosis subject is epithelial ovarian carcinoma when the detected value of the expression amount of the IGFBP4 gene, which is obtained by the detection device 100, is lower than the normal value stored in the storage device 200. Note that the expression amount of the IGFBP4 gene includes not only the expression amount of IGFBP4 itself but also an expression amount of a gene (DNAs or RNAs) that codes the IGFBP4.

In the nucleotide chip disposed in the detection device 100, polynucleotide complementary to a part or entirety of the gene that codes the IGFBP4 is fixed as a probe to a substrate. When genes extracted from the ovarian tissue as the diagnosis subject are dropped onto the nucleotide chip, only the gene that codes the IGFBP4 is captured by the nucleotide chip. The detection device 100 obtains an amount of such an IGFBP4-coding gene, which is captured by the nucleotide chip, as the detected value of the expression amount of the IGFBP4 gene.

The determination mechanism 301 of the CPU 300 receives, from the detection device 100, the detected value of the expression amount of the IGFBP4 gene derived from the ovarian tissue as the diagnosis subject, and reads out, from the storage device 200, the normal value of the expression amount of the IGFBP4 gene derived from the normal ovarian tissue. Moreover, the determination mechanism 301 compares the detected value of the expression amount of the IGFBP4 gene and the normal value of the expression amount of the IGFBP4 gene with each other, and determines that the ovarian tissue as the diagnosis subject is the epithelial ovarian carcinoma when the detected value is lower than the normal value. Reasons why it is determined that the ovarian tissue as the diagnosis subject is the epithelial ovarian carcinoma when the detected value is lower than the normal value will be described later.

### Fourteenth Embodiment

In a molecular diagnosis system of ovarian cancers according to a fourteenth embodiment, the detection device 100 shown in FIG. 1 obtains a detected value of an expression amount of a IGFBP6 gene in the ovarian tissue as the diagnosis subject, and the storage device 200 stores a normal value of the expression amount of the IGFBP6 gene in the normal ovarian tissue. Moreover, the determination mechanism 301 determines that the ovarian tissue as the diagnosis subject is epithelial ovarian carcinoma when the detected value of the expression amount of the IGFBP6 gene, which is obtained by the detection device 100, is lower than the normal value stored in the storage device 200. Note that the expression amount of the IGFBP6 gene includes not only the expression amount of IGFBP6 itself but also an expression amount of a gene (DNAs or RNAs) that codes the IGFBP6.

In the nucleotide chip disposed in the detection device 100, polynucleotide complementary to a part or entirety of the gene that codes the IGFBP6 is fixed as a probe to a substrate. When genes extracted from the ovarian tissue as the diagnosis subject are dropped onto the nucleotide chip, only the gene that codes the IGFBP6 is captured by the nucleotide chip. The detection device 100 obtains an amount of such an IGFBP6-coding gene, which is captured by the nucleotide chip, as the detected value of the expression amount of the IGFBP6 gene.

The determination mechanism 301 of the CPU 300 receives, from the detection device 100, the detected value of the expression amount of the IGFBP6 gene derived from the ovarian tissue as the diagnosis subject, and reads out, from the storage device 200, the normal value of the expression amount of the IGFBP6 gene derived from the normal ovarian tissue. Moreover, the determination mechanism 301 compares the detected value of the expression amount of the IGFBP6 gene and the normal value of the expression amount of the IGFBP6 gene with each other, and determines that the ovarian tissue as the diagnosis subject is the epithelial ovarian carcinoma when the detected value is lower than the normal value. Reasons why it is determined that the ovarian tissue as the diagnosis subject is the epithelial ovarian carcinoma when the detected value is lower than the normal value will be described later.

### Fifteenth Embodiment

In a molecular diagnosis system of ovarian cancers according to a fifteenth embodiment, the detection device 100 shown in FIG. 1 obtains a detected value of an expression amount of an INHA gene in the ovarian tissue as the diagnosis subject, and the storage device 200 stores a normal value of the expression amount of the INHA gene in the normal ovarian tissue. Moreover, the determination mechanism 301 determines that the ovarian tissue as the diagnosis subject is epithelial ovarian carcinoma when the detected value of the expression amount of the INHA gene, which is obtained by the detection device 100, is lower than the normal value stored in the storage device 200. Note that the expression amount of the INHA gene includes not only the expression amount of INHA itself but also an expression amount of a gene (DNAs or RNAs) that codes the INHA.

In the nucleotide chip disposed in the detection device 100, polynucleotide complementary to a part or entirety of the gene that codes the INHA is fixed as a probe to a substrate. When genes extracted from the ovarian tissue as the diagnosis subject are dropped onto the nucleotide chip, only the gene that codes the INHA is captured by the nucleotide chip. The detection device 100 obtains an amount of such an INHA-coding gene, which is captured by the nucleotide chip, as the detected value of the expression amount of the INHA gene.

The determination mechanism 301 of the CPU 300 receives, from the detection device 100, the detected value of the expression amount of the INHA gene derived from the ovarian tissue as the diagnosis subject, and reads out, from the storage device 200, the normal value of the expression amount of the INHA gene derived from the normal ovarian tissue. Moreover, the determination mechanism 301 compares the detected value of the expression amount of the INHA gene and the normal value of the expression amount of the INHA gene with each other, and determines that the ovarian tissue as the diagnosis subject is the epithelial ovarian carcinoma when the detected value is lower than the normal value. Reasons why it is determined that the ovarian tissue as the diagnosis subject is the epithelial ovarian carcinoma when the detected value is lower than the normal value will be described later.

### Sixteenth Embodiment

In a molecular diagnosis system of ovarian cancers according to a sixteenth embodiment, the detection device 100 shown in FIG. 1 obtains a detected value of an expression amount of a KRT7 gene in the ovarian tissue as the diagnosis subject, and the storage device 200 stores a normal value of the expression amount of the KRT7 gene in the normal ovarian tissue. Moreover, the determination mechanism 301 determines that the ovarian tissue as the diagnosis subject is epithelial ovarian carcinoma when the detected value of the expression amount of the KRT7 gene, which is obtained by the detection device 100, is higher than the normal value stored in the storage device 200. Note that the expression amount of the KRT7 gene includes not only the expression amount of KRT7 itself but also an expression amount of a gene (DNAs or RNAs) that codes the KRT7.

In the nucleotide chip disposed in the detection device 100, polynucleotide complementary to a part or entirety of the gene that codes the KRT7 is fixed as a probe to a substrate. When genes extracted from the ovarian tissue as the diagnosis subject are dropped onto the nucleotide chip, only the gene that codes the KRT7 is captured by the nucleotide chip. The detection device 100 obtains an amount of such a KRT7-coding gene, which is captured by the nucleotide chip, as the detected value of the expression amount of the KRT7 gene.

The determination mechanism 301 of the CPU 300 receives, from the detection device 100, the detected value of the expression amount of the KRT7 gene derived from the ovarian tissue as the diagnosis subject, and reads out, from the storage device 200, the normal value of the expression amount of the KRT7 gene derived from the normal ovarian tissue. Moreover, the determination mechanism 301 compares the detected value of the expression amount of the KRT7 gene and the normal value of the expression amount of the KRT7 gene with each other, and determines that the ovarian tissue as the diagnosis subject is the epithelial ovarian carcinoma when the detected value is higher than the normal value. Reasons why it is determined that the ovarian tissue as the diagnosis subject is the epithelial ovarian carcinoma when the detected value is higher than the normal value will be described later.

### Seventeenth Embodiment

In a molecular diagnosis system of ovarian cancers according to a seventeenth embodiment, the detection device 100 shown in FIG. 1 obtains a detected value of an expression amount of a LAMA2 gene in the ovarian tissue as the diagnosis subject, and the storage device 200 stores a normal value of the expression amount of the LAMA2 gene in the normal ovarian tissue. Moreover, the determination mechanism 301 determines that the ovarian tissue as the diagnosis subject is epithelial ovarian carcinoma when the detected value of the expression amount of the LAMA2 gene, which is obtained by the detection device 100, is lower than the normal value stored in the storage device 200. Note that the expression amount of the LAMA2 gene includes not only the expression amount of LAMA2 itself but also an expression amount of a gene (DNAs or RNAs) that codes the LAMA2.

In the nucleotide chip disposed in the detection device 100, polynucleotide complementary to a part or entirety of the gene that codes the LAMA2 is fixed as a probe to a substrate. When genes extracted from the ovarian tissue as the diagnosis subject are dropped onto the nucleotide chip, only the gene that codes the LAMA2 is captured by the nucleotide chip. The detection device 100 obtains an amount of such a LAMA2-coding gene, which is captured by the nucleotide chip, as the detected value of the expression amount of the LAMA2 gene.

The determination mechanism 301 of the CPU 300 receives, from the detection device 100, the detected value of the expression amount of the LAMA2 gene derived from the ovarian tissue as the diagnosis subject, and reads out, from the storage device 200, the normal value of the expression amount of the LAMA2 gene derived from the normal ovarian tissue. Moreover, the determination mechanism 301 compares the detected value of the expression amount of the LAMA2 gene and the normal value of the expression amount of the LAMA2 gene with each other, and determines that the ovarian tissue as the diagnosis subject is the epithelial ovarian carcinoma when the detected value is lower than the normal value. Reasons why it is determined that the ovarian tissue as the diagnosis subject is the epithelial ovarian carcinoma when the detected value is lower than the normal value will be described later.

### Eighteenth Embodiment

In a molecular diagnosis system of ovarian cancers according to a eighteenth embodiment, the detection device 100 shown in FIG. 1 obtains a detected value of an expression amount of a MMP2 gene in the ovarian tissue as the diagnosis subject, and the storage device 200 stores a normal value of the expression amount of the MMP2 gene in the normal ovarian tissue. Moreover, the determination mechanism 301 determines that the ovarian tissue as the diagnosis subject is mucinous adenocarcinoma, clear cell adenocarcinoma or serous adenocarcinoma when the detected value of the expression amount of the MMP2 gene, which is obtained by the detection device 100, is lower than the normal value stored in the storage device 200. Note that the expression amount of the MMP2 gene includes not only the expression amount of MMP2 itself but also an expression amount of a gene (DNAs or RNAs) that codes the MMP2.

In the nucleotide chip disposed in the detection device 100, polynucleotide complementary to a part or entirety of the gene that codes the MMP2 is fixed as a probe to a substrate. When genes extracted from the ovarian tissue as the diagnosis subject are dropped onto the nucleotide chip, only the gene that codes the MMP2 is captured by the nucleotide chip. The detection device 100 obtains an amount of such a MMP2-coding gene, which is captured by the nucleotide chip, as the detected value of the expression amount of the MMP2 gene.

The determination mechanism 301 of the CPU 300 receives, from the detection device 100, the detected value of the expression amount of the MMP2 gene derived from the ovarian tissue as the diagnosis subject, and reads out, from the storage device 200, the normal value of the expression amount of the MMP2 gene derived from the normal ovarian tissue. Moreover, the determination mechanism 301 compares the detected value of the expression amount of the MMP2 gene and the normal value of the expression amount of the MMP2 gene with each other, and determines that the ovarian tissue as the diagnosis subject is the mucinous adenocarcinoma, the clear cell adenocarcinoma or the serous adenocarcinoma when the detected value is lower than the normal value. Reasons why it is determined that the ovarian tissue as the diagnosis subject is the mucinous adenocarcinoma, the clear cell adenocarcinoma or the serous adenocarcinoma when the detected value is lower than the normal value will be described later.

### Nineteenth Embodiment

In a molecular diagnosis system of ovarian cancers according to a nineteenth embodiment, the detection device 100 shown in FIG. 1 obtains a detected value of an expression amount of a TIMP1 gene in the ovarian tissue as the diagnosis subject, and the storage device 200 stores a normal value of the expression amount of the TIMP1 gene in the normal ovarian tissue. Moreover, the determination mechanism 301 determines that the ovarian tissue as the diagnosis subject is epithelial ovarian carcinoma when the detected value of the expression amount of the TIMP1 gene, which is obtained by the detection device 100, is lower than the normal value stored in the storage device 200. Note that the expression amount of the TIMP1 gene includes not only the expression amount of TIMP1 itself but also an expression amount of a gene (DNAs or RNAs) that codes the TIMP1.

In the nucleotide chip disposed in the detection device 100, polynucleotide complementary to a part or entirety of the gene that codes the TIMP1 is fixed as a probe to a substrate. When genes extracted from the ovarian tissue as the diagnosis subject are dropped onto the nucleotide chip, only the gene that codes the TIMP1 is captured by the nucleotide chip. The detection device 100 obtains an amount of such a TIMP1-coding gene, which is captured by the nucleotide chip, as the detected value of the expression amount of the TIMP1 gene.

The determination mechanism 301 of the CPU 300 receives, from the detection device 100, the detected value of the expression amount of the TIMP1 gene derived from the ovarian tissue as the diagnosis subject, and reads out, from the storage device 200, the normal value of the expression amount of the TIMP1 gene derived from the normal ovarian tissue. Moreover, the determination mechanism 301 compares the detected value of the expression amount of the TIMP1 gene and the normal value of the expression amount of the TIMP1 gene with each other, and determines that the ovarian tissue as the diagnosis subject is the epithelial ovarian carcinoma when the detected value is lower than the normal value. Reasons why it is determined that the ovarian tissue as the diagnosis subject is the epithelial ovarian carcinoma when the detected value is lower than the normal value will be described later.

### EXAMPLES

A description will be made below in detail of Examples which support such determination criteria of the determination mechanisms 301 of the molecular diagnosis systems of the ovarian cancers according to the above-described first to nineteenth embodiments.

(Preparation of Samples)

20 ovarian malignancy tissues were extirpated from patients, from who informed consent was obtained, among Japanese patients diagnosed to have the ovarian cancer at the Department of Obstetrics and Gynecology at the School of Medicine in Keio University. For the extirpated ovarian malignancy tissues, tissue types thereof were diagnosed pathologically by the Department of Clinical Pathology at the Central Clinical Laboratory and the Department of Obstetrics and Gynecology in Keio University. Then, the extirpated ovarian malignancy tissues were classified into seven serous adenocarcinomas, four endometrioid adenocarcinomas, two mucinous adenocarcinomas, and seven clear cell adenocarcinomas.

Next, each of the ovarian malignancy tissues was put into a mortar into which liquid nitrogen was filled, and was then finely milled by a pestle. The milled ovarian malignancy tissue was put into a solution (RNAlaer-ICE Kit (registered trademark), made by Ambion Inc.) containing an inhibitor of ribonuclease, and the solution was made to penetrate the ovarian malignancy tissue. Thereafter, a resultant thus obtained was stored at −80° C. Moreover, before extracting the RNA, the solution containing the inhibitor of the ribonuclease was made to fully penetrate the ovarian malignancy tissue at −20° C. for 24 hours.

Next, by using a RNA extraction kit (RNAqueous Kit (registered trademark), made by Ambion Inc.), the total RNA was extracted from the ovarian malignancy tissue. The DNA mixed in the extracted total RNA was decomposed by deoxyribonuclease (Turbo DNA free kit (registered trademark), made by Ambion Inc.), and the total RNA was condensed by the ethanol precipitation method. Thereafter, by electrophoresis, purity of the total RNA and the presence of 28s ribosome RNA and 18s ribosome RNA were recognized. Moreover, a concentration of the total RNA was recognized by a spectrophotometer (BioSpec Mini (registered trademark), made by Shimadzu Corporation).

Next, 1 μg of the total RNA was used as a template, and cDNA was synthesized from mRNA contained in the total RNA by using an amplification kit (Message Amp II Biotin Enhanced (registered trademark), made by Ambion Inc.). Moreover, a large number of biotinylated anti-sense RNAs having sequences complementary to mRNA were amplified from cDNA.

(Preparation of Probes)

Five polynucleotide sequences for detecting the anti-sense RNA of the mRNA of the ApoA1_{—}1 gene were designed by sequence design software (made by CombiMatrix Corporation). Five polynucleotides for detecting the anti-sense RNA were also designed for each of the ApoE_{—}2 gene, the ApoJ_{—}1 gene, the ARL-1_{—}1 gene, the BST2_{—}1 gene, the CCNE1_{—}1 gene, the CDK4 gene, the CTNNB1 gene, the ERBB2_{—}1 gene, the ESR1 gene, the HOST2 gene, the HSD17B1 gene, the IGFBP4 gene, the IGFBP6 gene, the INHA gene, the KRT7_{—}1 gene, the LAMA2 gene, the MMP2 gene, and the TIMP1_{—}1 gene. Thereafter, polynucleotides complementary to other polynucleotides, and polynucleotides in which sequences are approximate to the other polynucleotides were excluded, and three polynucleotides were selected as probes from the five polynucleotides for each of the genes. Microarrays having the selected probes were manufactured by the phosphoamidite method in CombiMatrix Corporation.

(Method for Assay)

First, each of the probes was disposed on a CustomArray (registered trademark) of CombiMatrix Corporation, and was hybridized with 2 μg of anti-sense RNA derived from each of the mucinous adenocarcinomas, the clear cell adenocarcinomas, the endometrioid adenocarcinomas, and the serous adenocarcinomas. Moreover, as reference controls, biotinylated anti-sense RNA prepared from RNA (made by Clontech Laboratories Inc.) derived from normal ovarian tissues and the probes were hybridized with each other. Note that the RNA of Clontech Laboratories Inc., which was derived from the normal ovarian tissues, was one gathered from 15 Caucasian women.

After such hybridization, the CustomArray was washed in accordance with a manual of CombiMatrix Corporation, and each of the unreacted probes was blocked. Thereafter, the biotinylated RNA was labeled by Cy3-tagged streptavidin. Next, fluorescence of Cy3 was read by a scanner (GenePix4000B, made by Axon Instruments, a division of MDS Inc.), and a TIFF image was created. Moreover, the TIFF image was analyzed by image analysis software (Imager, made by CombiMatrix Corporation), a fluorescence intensity of the Cy3 was converted into numeric values, and a text file was created. Furthermore, the text file was captured into microarray data analysis software (GeneSpring GX 7.3.1, made by Tomy Digital Biology Co., Ltd.), and the entire data of the fluorescence intensity of the Cy3 was normalized in order to standardize the data for each CustomArray. Thereafter, the numeric values of the fluorescence intensity of the Cy3 were normalized so that a fluorescence intensity of the Cy3 in the reference control could become 1. Note that the GeneSpring GX 7.3.1 is provided with a function to normalize the data so that expression data in the normal tissue can become 1.0. Hereinafter, the normalized and standardized fluorescence intensity of the Cy3 is defined as a detected intensity of the anti-sense RNA of the gene in each of Examples.

(Assay of ApoA1_{—}1 Gene Expression)

A first probe for detecting the anti-sense RNA of the ApoA1_{—}1 gene is composed of the base sequence of the sequence number 2, which is described in the sequence table, a second probe for the above-described purpose is composed of the base sequence of the sequence number 3, which is described therein, and a third probe for the above-described purpose is composed of the base sequence of the sequence number 4, which is described therein. The first probe corresponds to the 155-th to 189-th nucleotides of the ApoA1_{—}1 gene composed of the base sequence of the sequence number 1. The second probe corresponds to the 234-th to 268-th nucleotides of the ApoA1_{—1} gene, and the third probe corresponds to the 795-th to 829-th nucleotides of the ApoA1_{—}1 gene.

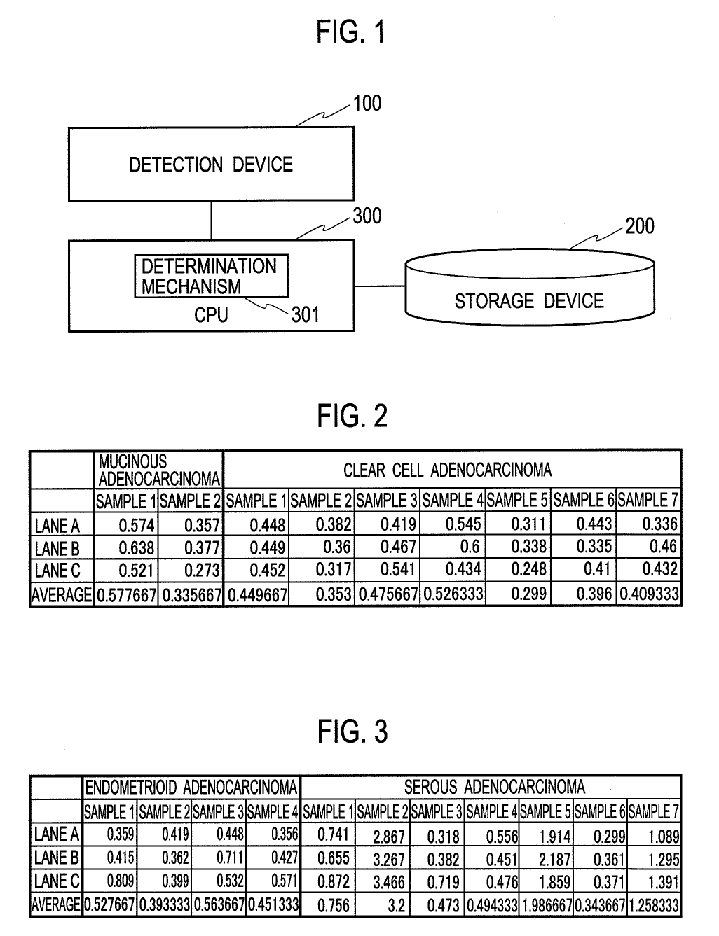

FIG. 2 and FIG. 3 show the detected intensities of the anti-sense RNAs in the respective lanes of the CustomArrays in the case of using the first probe. Here, the number of lanes is three for each of the samples. Heretofore, while it has been considered that the expression amount of the ApoA1 in the serum of the ovarian cancer patient is extremely small, the expression amount for each of the tissue types is unknown, and there has been no report thereof in the clear cell adenocarcinoma. As opposed to this, in the case of using the first probe, the detected intensities of the anti-sense RNAs in the ApoA1_{—}1 genes derived from the clear cell adenocarcinoma were lower than the detected intensities of the RNAs derived from the adenocarcinomas of the other tissue types. Hence, when the amount of such examination-subject RNA to be hybridized with the first probe is small, it is possible to diagnose that the tissue from which the examination-subject RNA is derived is the clear cell adenocarcinoma.

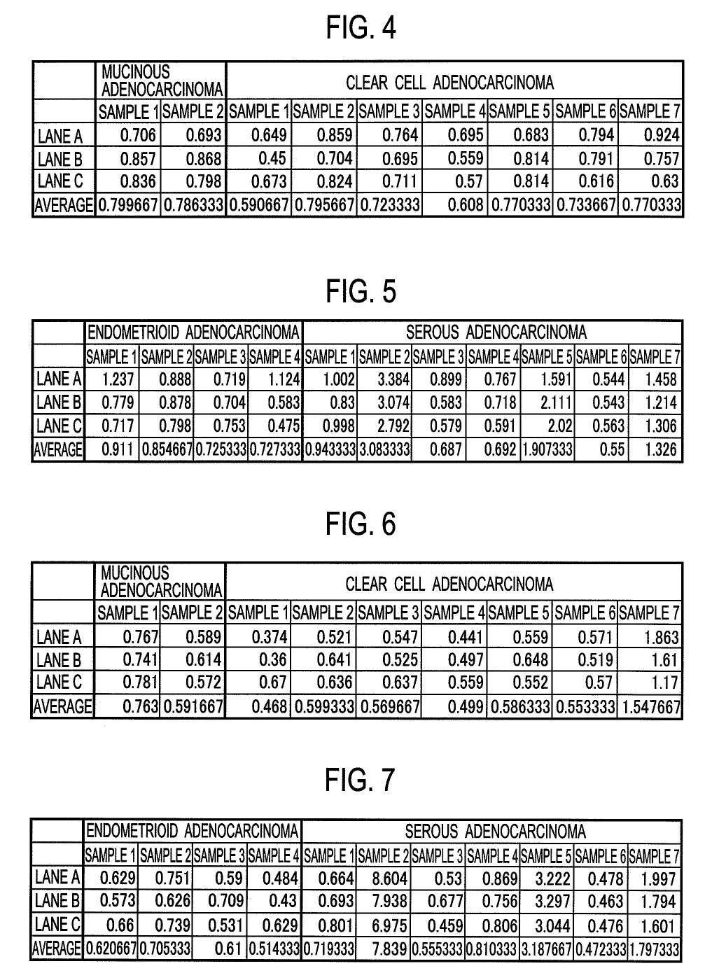

FIG. 4 and FIG. 5 show the detected intensities of the anti-sense RNAs in the case of using the second probe, and FIG. 6 and FIG. 7 show the detected intensities of the anti-sense RNAs in the case of using the third probe. Also in the case of using the second and third probes, the detected intensities of the anti-sense RNAs of the ApoA1_{—}1 gene derived from the clear cell adenocarcinoma were lower than the detected intensities of the RNAs derived from the adenocarcinomas of the other tissue types.

(Assay of ApoE_{—}2 Gene Expression)

A first probe for detecting the anti-sense RNA of the ApoE_{—}2 gene is composed of the base sequence of the sequence number 6, a second probe for the above-described purpose is composed of the base sequence of the sequence number 7, and a third probe for the above-described purpose is composed of the base sequence of the sequence number 8. The first probe corresponds to the 309-th to 343-rd nucleotides of the ApoE_{—}2 gene composed of the base sequence of the sequence number 5. The second probe corresponds to the 335-th to 369-th nucleotides of the ApoE_{—}2 gene, and the third probe corresponds to the 1103-rd to 1137-th nucleotides of the ApoE_{—}2 gene.

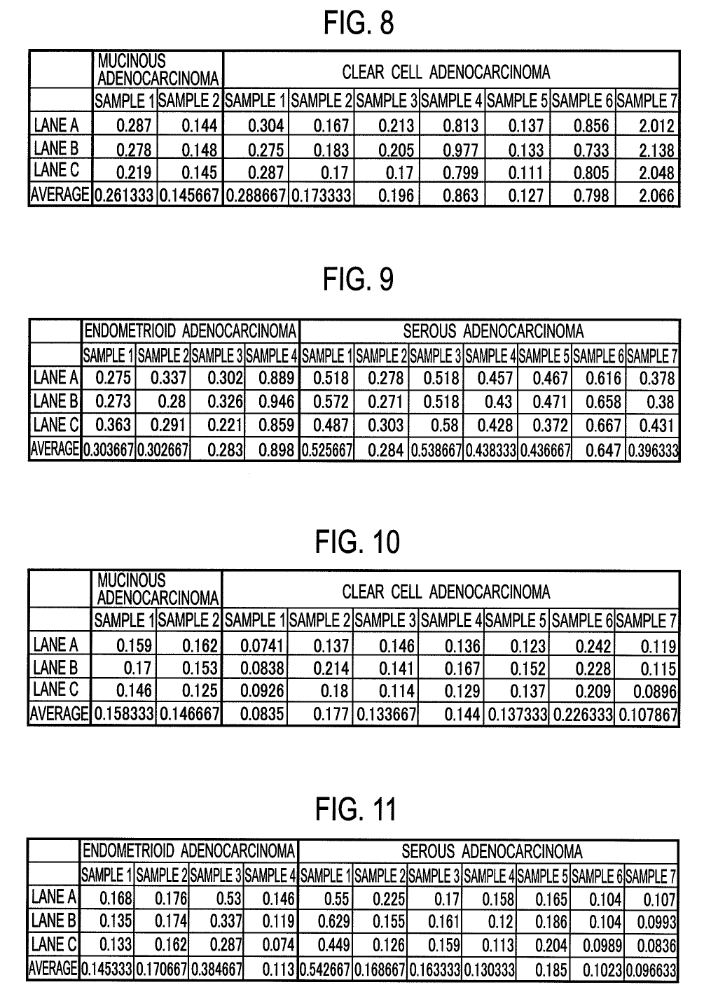

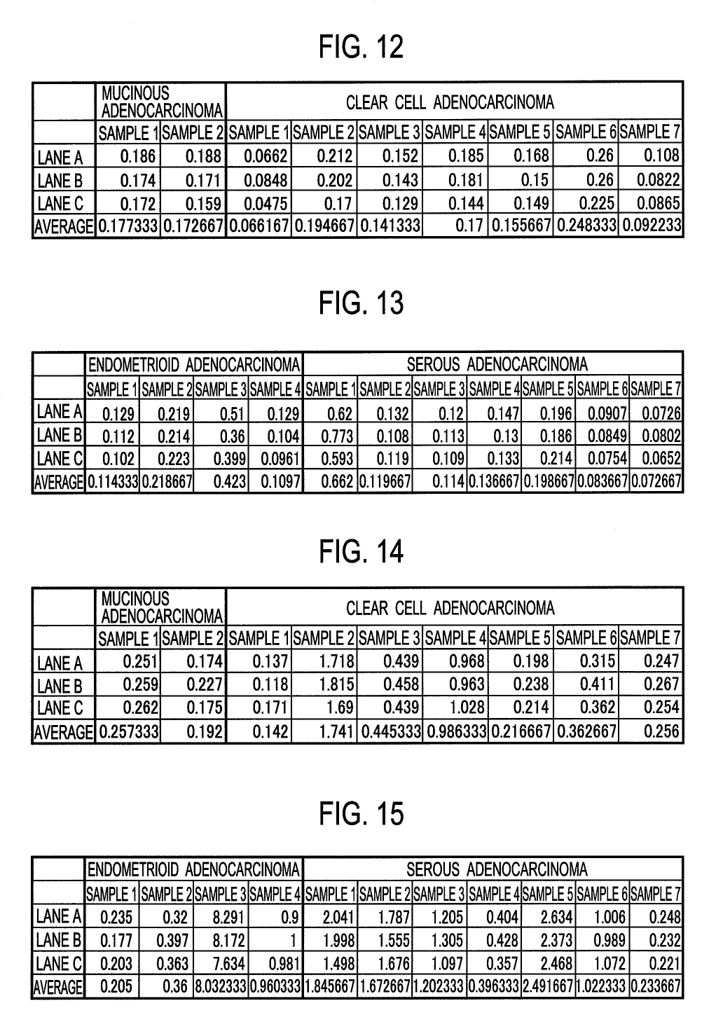

FIG. 8 and FIG. 9 show the detected intensities of the anti-sense RNAs of the ApoE_{—}2 gene in the case of using the first probe, FIG. 10 and FIG. 11 show the detected intensities concerned in the case of using the second probe, and FIG. 12 and FIG. 13 show the detected intensities concerned in the case of using the third probe. Heretofore, it has been considered that the expression amount of the ApoE_{—}2 is increased in the ovarian cancer patient. Moreover, the expression amount of the ApoE_{—}2 for each of the tissue types has not been reported. As opposed to this, it was shown in the Example that, in all of the tissue types of the mucinous adenocarcinoma, the clear cell adenocarcinoma, the endometrioid adenocarcinoma and the serous adenocarcinoma, the expression amounts of the ApoE_{—}2 gene became lower than that in the normal ovarian tissue. Hence, when such an amount of the ApoE_{—}2 gene is low, it is possible to diagnose that the tissue concerned is the epithelial ovarian carcinoma.

(Assay of ApoJ_{—}1 Gene Expression)

A first probe for detecting the anti-sense RNA of the ApoJ_{—}1 gene is composed of the base sequence of the sequence number 10, a second probe for the above-described purpose is composed of the base sequence of the sequence number 11, and a third probe for the above-described purpose is composed of the base sequence of the sequence number 12. The first probe corresponds to the 737-th to 772-nd nucleotides of the ApoJ_{—}1 gene composed of the base sequence of the sequence number 9. The second probe corresponds to the 811-st to 845-th nucleotides of the ApoJ_{—}1 gene, and the third probe corresponds to the 1033-rd to 1067-th nucleotides of the ApoJ_{—}1 gene.

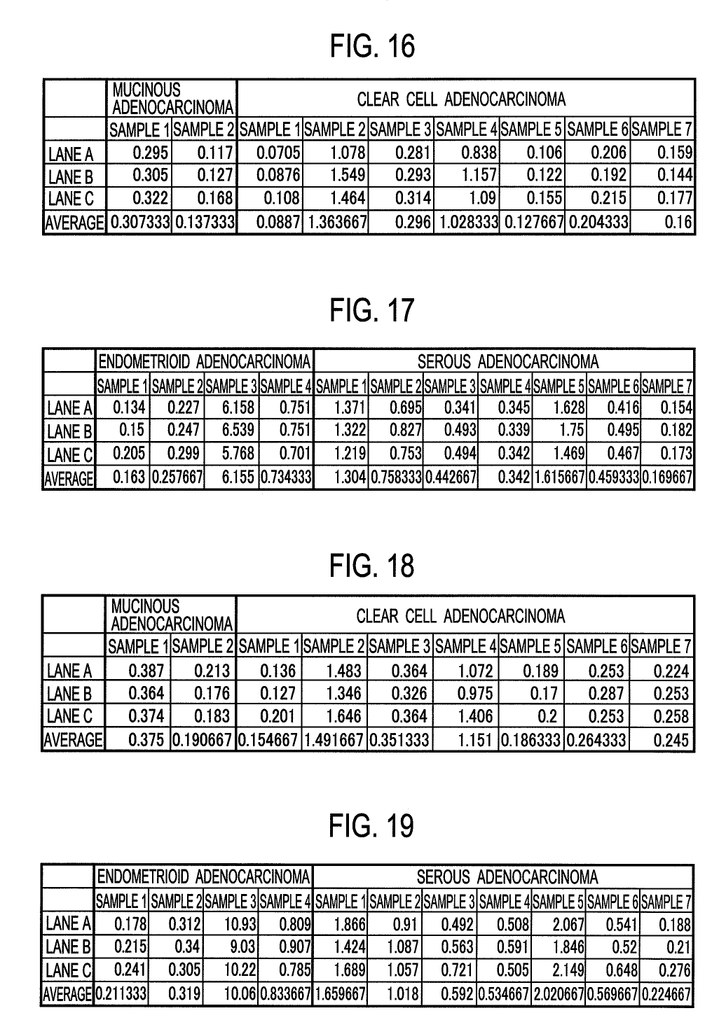

FIG. 14 and FIG. 15 show the detected intensities of the anti-sense RNAs of the ApoJ_{—}1 gene in the case of using the first probe, FIG. 16 and FIG. 17 show the detected intensities concerned in the case of using the second probe, and FIG. 18 and FIG. 19 show the detected intensities concerned in the case of using the third probe. Heretofore, it has been considered that the expression amount of the ApoJ_{—}1 is increased in the ovarian cancer patient. Moreover, the expression amount of the ApoJ_{—}1 for each of the tissue types has not been reported. As opposed to this, it was shown in the Example that, though the expression amount of the ApoJ_{—}1 gene was varied in the endometrioid adenocarcinoma and the serous adenocarcinoma, the expression amounts of the ApoJ_{—}1 gene in the mucinous adenocarcinoma and the clear cell adenocarcinoma became lower than that in the normal ovarian tissue. Hence, when such an amount of the ApoJ_{—}1 gene is low, it is possible to diagnose that the tissue concerned is the mucinous adenocarcinoma or the clear cell adenocarcinoma.

(Assay of ARL-1_{—}1 Gene Expression)

A first probe for detecting the anti-sense RNA of the ARL-1_{—}1 gene is composed of the base sequence of the sequence number 14, a second probe for the above-described purpose is composed of the base sequence of the sequence number 15, and a third probe for the above-described purpose is composed of the base sequence of the sequence number 16. The first probe corresponds to the 567-th to 602-nd nucleotides of the ARL-1_{—}1 gene composed of the base sequence of the sequence number 13. The second probe corresponds to the 802-nd to 836-th nucleotides of the ARL-1_{—}1 gene, and the third probe corresponds to the 1320-th to 1357-th nucleotides of the ARL-1_{—}1 gene.

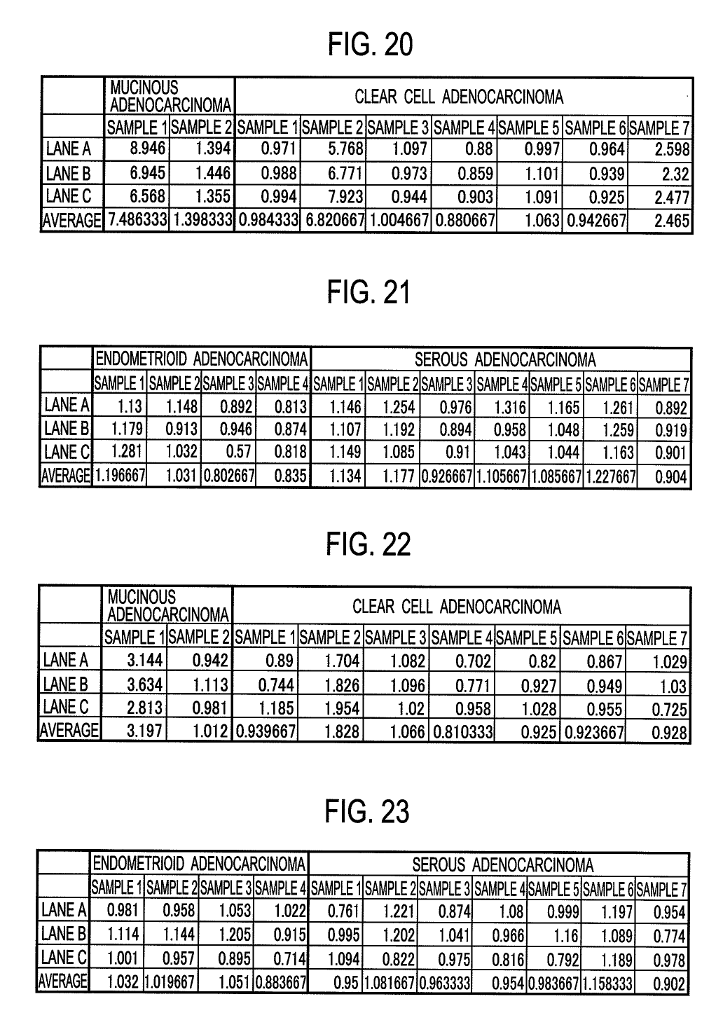

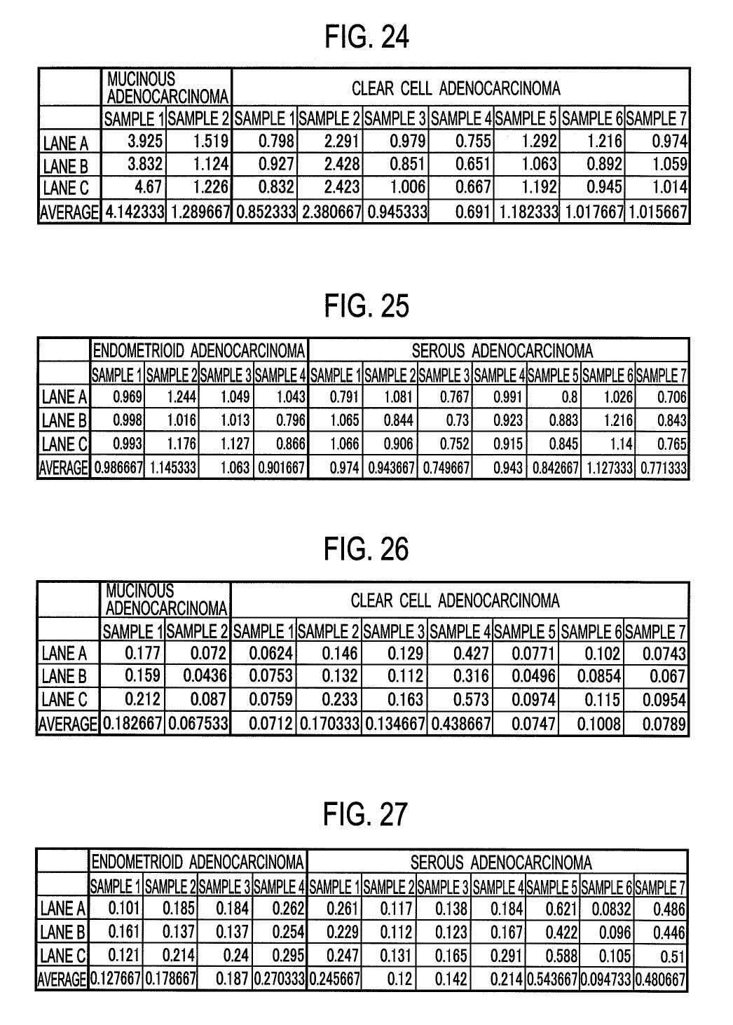

FIG. 20 and FIG. 21 show the detected intensities of the anti-sense RNAs of the ARL-1_{—}1 gene in the case of using the first probe, FIG. 22 and FIG. 23 show the detected intensities concerned in the case of using the second probe, and FIG. 24 and FIG. 25 show the detected intensities concerned in the case of using the third probe. Heretofore, the expression amount of the ARL-1_{—}1 in the ovarian tumor has not been reported. As opposed to this, it was shown in the Example that the expression amount of the ARL-1_{—}1 gene in the mucinous adenocarcinoma became higher than that in the normal ovarian tissue. Hence, when such an amount of the ARL-1_{—}1 gene is high, it is possible to diagnose that the tissue concerned is the mucinous adenocarcinoma.

(Assay of BST2_{—}1 Gene Expression)

A first probe for detecting the anti-sense RNA of the BST2_{—}1 gene is composed of the base sequence of the sequence number 18, a second probe for the above-described purpose is composed of the base sequence of the sequence number 19, and a third probe for the above-described purpose is composed of the base sequence of the sequence number 20. The first probe corresponds to the 11-th to 45-th nucleotides of the BST2_{—}1 gene composed of the base sequence of the sequence number 17. The second probe corresponds to the 186-th to 220-th nucleotides of the BST2_{—}1 gene, and the third probe corresponds to the 373-rd to 407-th nucleotides of the BST2_{—}1 gene.

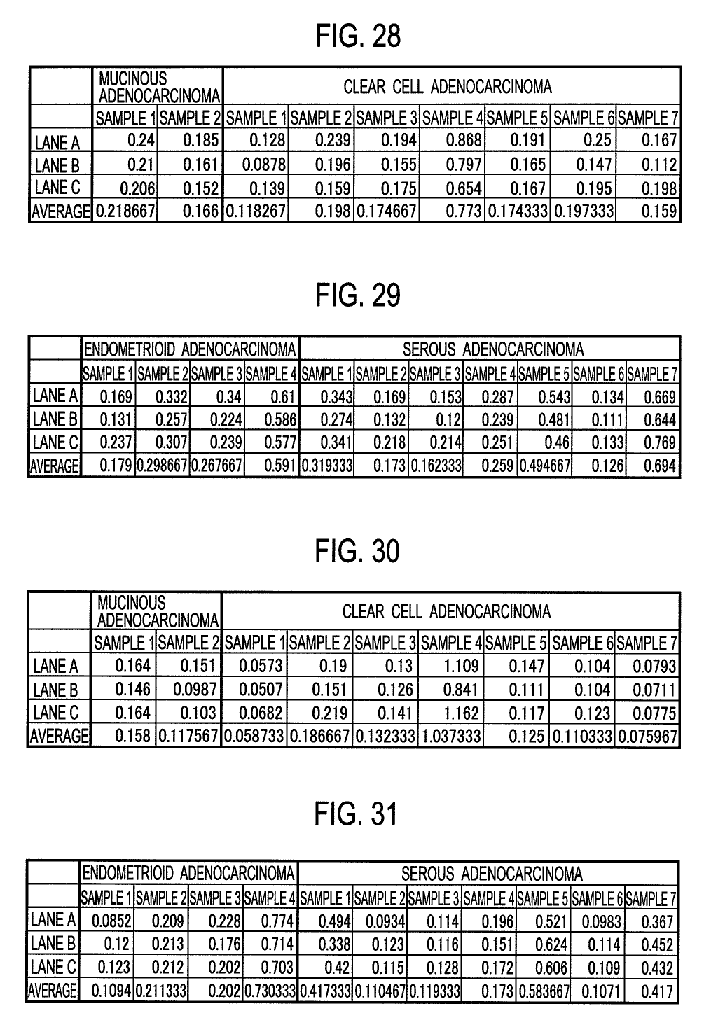

FIG. 26 and FIG. 27 show the detected intensities of the anti-sense RNAs of the BST2_{—}1 gene in the case of using the first probe, FIG. 28 and FIG. 29 show the detected intensities concerned in the case of using the second probe, and FIG. 30 and FIG. 31 show the detected intensities concerned in the case of using the third probe. Heretofore, it has been considered that the expression amount of the BST2_{—}1 is increased in the ovarian cancer patient. Moreover, the expression amount of the BST2_{—}1 for each of the tissue types has not been reported. As opposed to this, it was shown in the Example that, in all of the tissue types of the mucinous adenocarcinoma, the clear cell adenocarcinoma, the endometrioid adenocarcinoma and the serous adenocarcinoma, the expression amounts of the BST2_{—}1 gene became lower than that in the normal ovarian tissue. Hence, when such an amount of the BST2_{—}1 gene is low, it is possible to diagnose that the tissue concerned is the epithelial ovarian carcinoma.

(Assay of CCNE1_{—}1 Gene Expression)

A first probe for detecting the anti-sense RNA of the CCNE1_{—}1 gene is composed of the base sequence of the sequence number 22, a second probe for the above-described purpose is composed of the base sequence of the sequence number 23, and a third probe for the above-described purpose is composed of the base sequence of the sequence number 24. The first probe corresponds to the 910-th to 945-nd nucleotides of the CCNE1_{—}1 gene composed of the base sequence of the sequence number 21. The second probe corresponds to the 956-th to 990-th nucleotides of the CCNE1_{—}1 gene, and the third probe corresponds to the 1484-th to 1518-th nucleotides of the CCNE1_{—}1 gene.

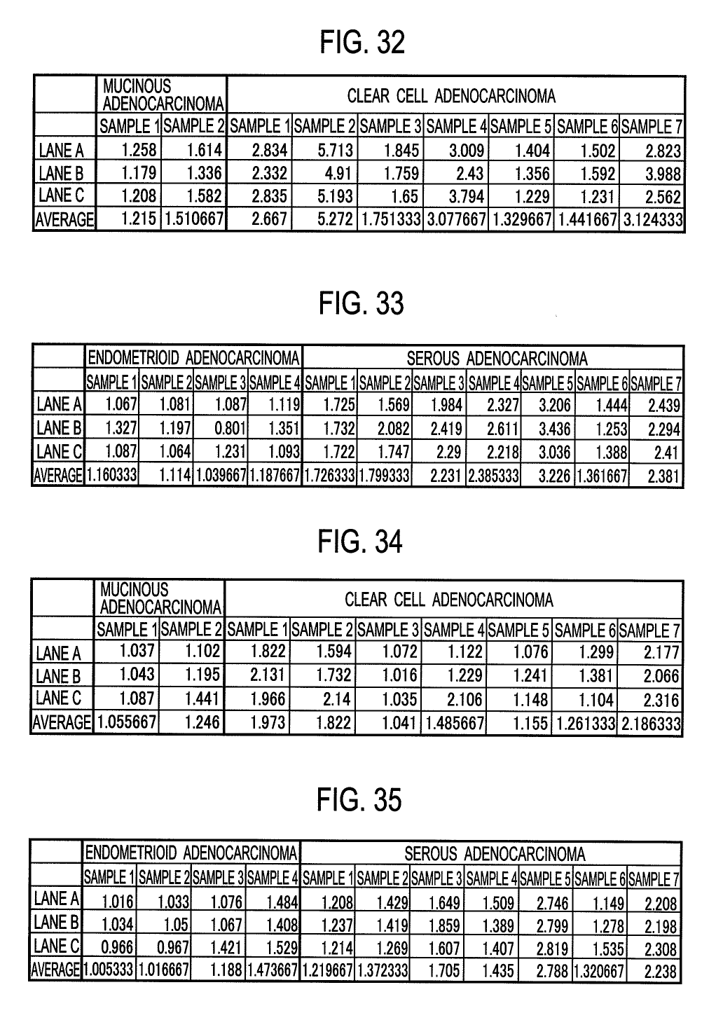

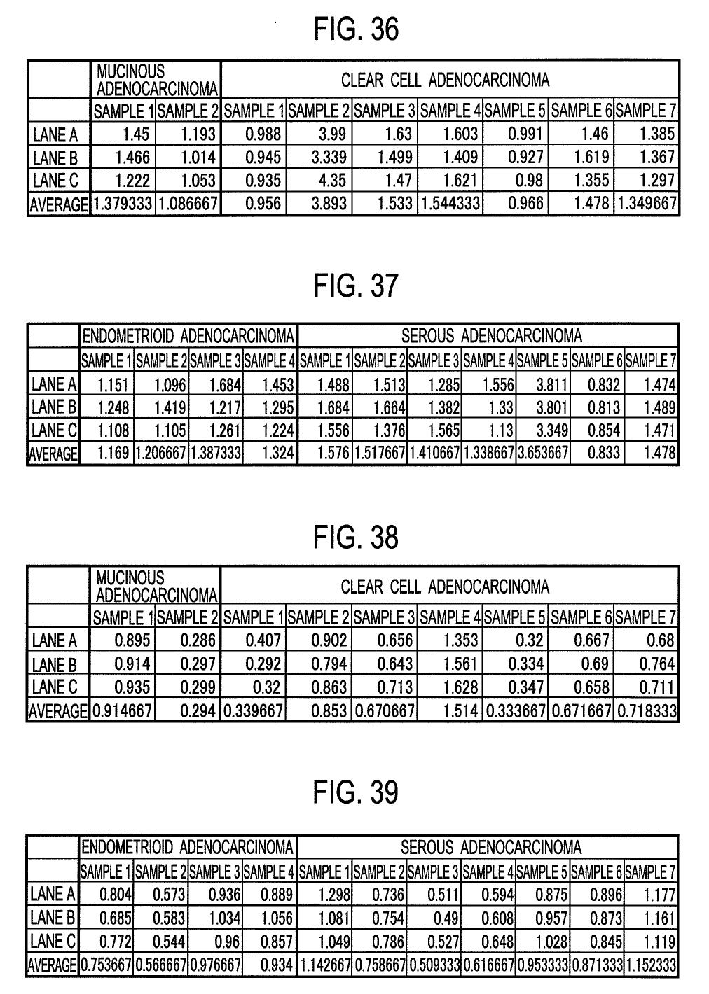

FIG. 32 and FIG. 33 show the detected intensities of the anti-sense RNAs of the CCNE1_{—}1 gene in the case of using the first probe, FIG. 34 and FIG. 35 show the detected intensities concerned in the case of using the second probe, and FIG. 36 and FIG. 37 show the detected intensities concerned in the case of using the third probe. Heretofore, it has been considered that the expression amount of the CCNE1_{—}1 is decreased in the ovarian cancer patient. Moreover, the expression amount of the CCNE1_{—}1 for each of the tissue types has not been reported. As opposed to this, it was shown in the Example that the expression amounts of the CCNE1_{—}1 gene in the clear cell adenocarcinoma and the serous adenocarcinoma became higher than that in the normal ovarian tissue. Hence, when such an amount of the CCNE1_{—}1 gene is high, it is possible to diagnose that the tissue concerned is the clear cell adenocarcinoma and the serous adenocarcinoma.

(Assay of CDK4 Gene Expression)

A first probe for detecting the anti-sense RNA of the CDK4 gene is composed of the base sequence of the sequence number 26, a second probe for the above-described purpose is composed of the base sequence of the sequence number 27, and a third probe for the above-described purpose is composed of the base sequence of the sequence number 28. The first probe corresponds to the 579-th to 613-rd nucleotides of the CDK4 gene composed of the base sequence of the sequence number 25. The second probe corresponds to the 731-st to 765-th nucleotides of the CDK4 gene, and the third probe corresponds to the 1115-th to 1149-th nucleotides of the CDK4 gene.

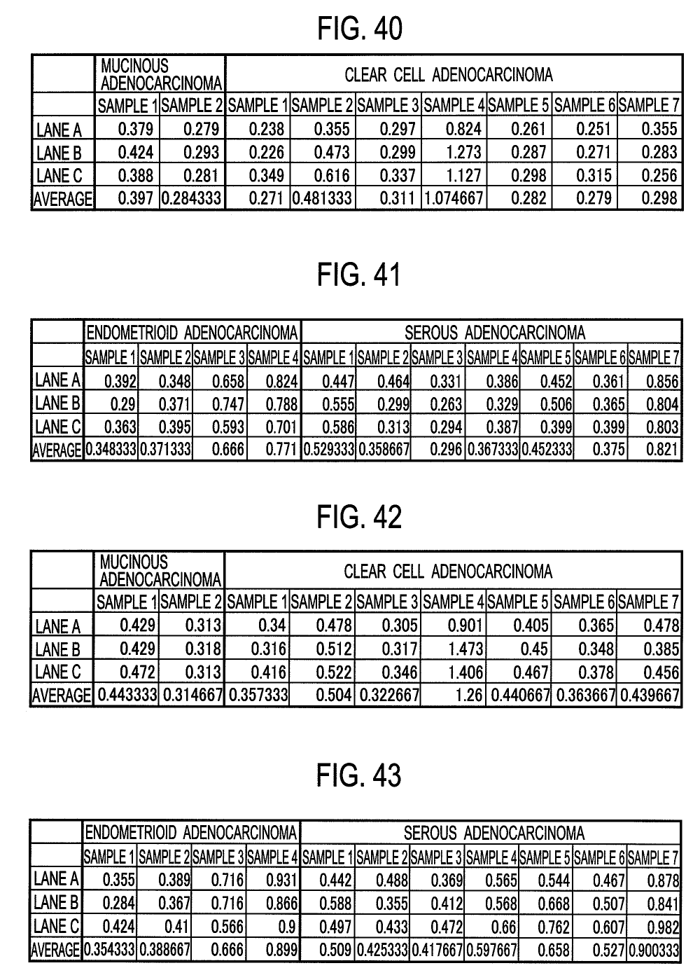

FIG. 38 and FIG. 39 show the detected intensities of the anti-sense RNAs of the CDK4 gene in the case of using the first probe, FIG. 40 and FIG. 41 show the detected intensities concerned in the case of using the second probe, and FIG. 42 and FIG. 43 show the detected intensities concerned in the case of using the third probe. Heretofore, it has been considered that the expression amount of the CDK4 is increased in the ovarian cancer patient. Moreover, the expression amount of the CDK4 for each of the tissue types has not been reported. As opposed to this, it was shown in the Example that, in all of the tissue types of the mucinous adenocarcinoma, the clear cell adenocarcinoma, the endometrioid adenocarcinoma and the serous adenocarcinoma, the expression amounts of the CDK4 gene became lower than that in the normal ovarian tissue. Hence, when such an amount of the CDK4 gene is low, it is possible to diagnose that the tissue concerned is the epithelial ovarian carcinoma.

(Assay of CTNNB1 Gene Expression)

A first probe for detecting the anti-sense RNA of the CTNNB1 gene is composed of the base sequence of the sequence number 30, a second probe for the above-described purpose is composed of the base sequence of the sequence number 31, which is described therein, and a third probe for the above-described purpose is composed of the base sequence of the sequence number 32. The first probe corresponds to the 2950-th to 2985-th nucleotides of the CTNNB1 gene composed of the base sequence of the sequence number 29. The second probe corresponds to the 3012-nd to 3046-th nucleotides of the CTNNB1 gene, and the third probe corresponds to the 3625-th to 3660-th nucleotides of the CTNNB1 gene.

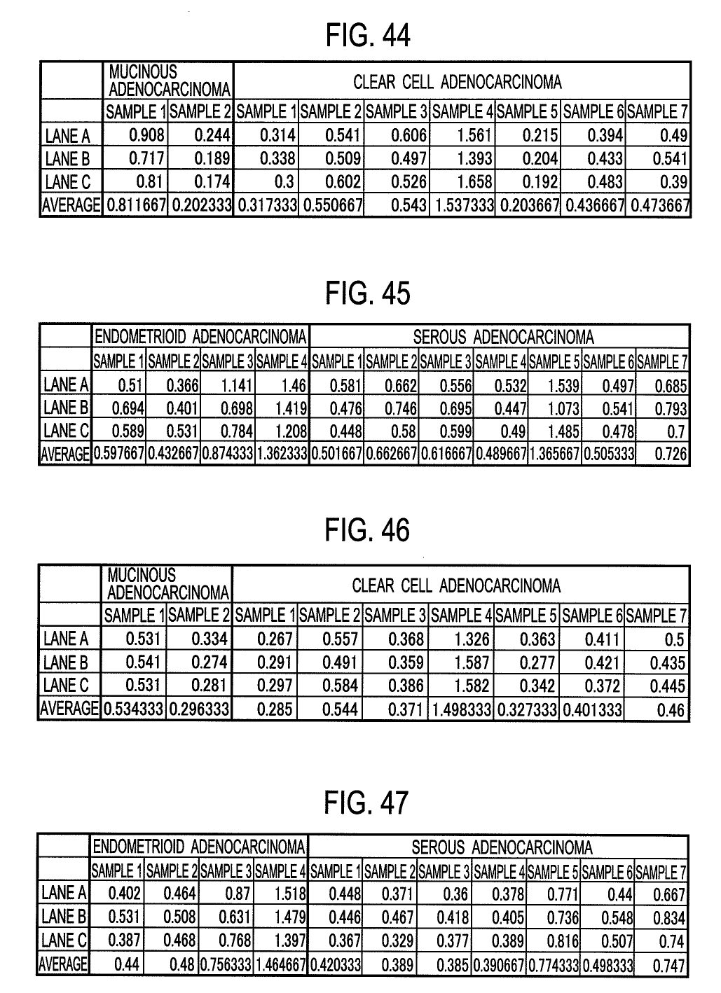

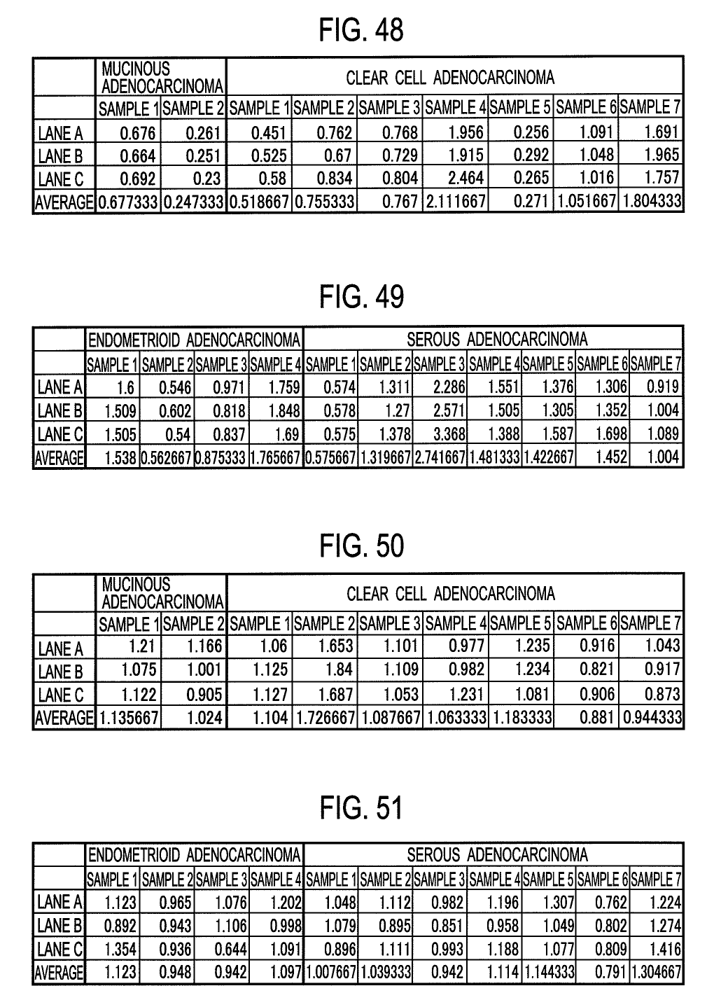

FIG. 44 and FIG. 45 show the detected intensities of the anti-sense RNAs of the CTNNB1 gene in the case of using the first probe, FIG. 46 and FIG. 47 show the detected intensities concerned in the case of using the second probe, and FIG. 48 and FIG. 49 show the detected intensities concerned in the case of using the third probe. Heretofore, it has been considered that the expression amount of the CTNNB1 is increased in the ovarian cancer. Moreover, the expression amount of the CTNNB1 for each of the tissue types has not been reported. As opposed to this, the detected intensities of the anti-sense RNAs in the CTNNB1 genes, which were detected by the first probe and the second probe, were lower than that in the normal ovarian tissue, in all of the tissue types. Hence, when the amount of such examination-subject RNA to be hybridized with the first probe and the second probe is small, it is possible to diagnose that the tissue from which the examination-subject RNA is derived is the epithelial ovarian carcinoma.

(Assay of ERBB2_{—}1 Gene Expression)

A first probe for detecting the anti-sense RNA of the ERBB2_{—}1 gene is composed of the base sequence of the sequence number 34, a second probe for the above-described purpose is composed of the base sequence of the sequence number 35, and a third probe for the above-described purpose is composed of the base sequence of the sequence number 36. The first probe corresponds to the 4074-th to 4108-th nucleotides of the ERBB2_{—}1 gene composed of the base sequence of the sequence number 33. The second probe corresponds to the 4495-th to 4529-th nucleotides of the ERBB2_{—}1 gene, and the third probe corresponds to the 4623-rd to 4657-th nucleotides of the ERBB2_{—}1 gene.

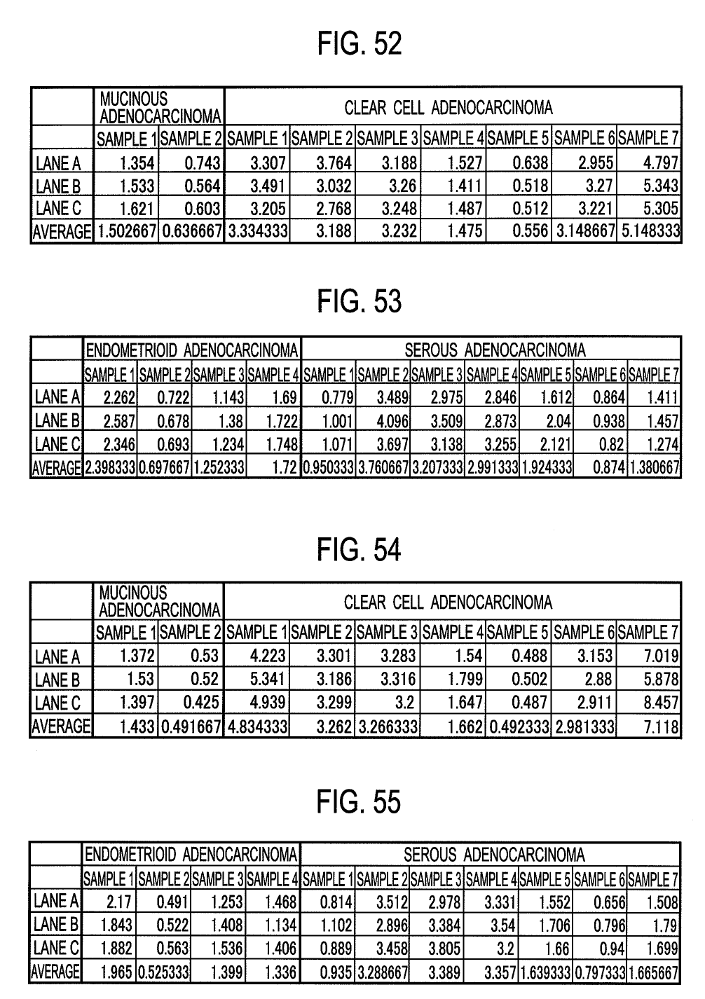

FIG. 50 and FIG. 51 show the detected intensities of the anti-sense RNAs of the ERBB2_{—}1 gene in the case of using the first probe, FIG. 52 and FIG. 53 show the detected intensities concerned in the case of using the second probe, and FIG. 54 and FIG. 55 show the detected intensities concerned in the case of using the third probe. Heretofore, it has been considered that the expression amount of the ERBB2_{—}1 is decreased in the ovarian cancer. Moreover, the expression amount of the ERBB2_{—}1 for each of the tissue types has not been reported. As opposed to this, it was shown in the Example that, in the case of using the second probe and the third probe, the detected intensities of the anti-sense RNAs in the ERBB2_{—}1 genes derived from the clear cell adenocarcinoma and the serous adenocarcinoma became higher than that in the normal ovarian tissue. Hence, when the amount of such examination-subject RNA to be hybridized with the second probe and the third probe is large, it is possible to diagnose that the tissue from which the examination-subject RNA is derived is the clear cell adenocarcinoma or the serous adenocarcinoma.

(Assay of ESR1 Gene Expression)

A first probe for detecting the anti-sense RNA of the ESR1 gene is composed of the base sequence of the sequence number 38, a second probe for the above-described purpose is composed of the base sequence of the sequence number 39, and a third probe for the above-described purpose is composed of the base sequence of the sequence number 40. The first probe corresponds to the 5681-st to 5715-th nucleotides of the ESR1 gene composed of the base sequence of the sequence number 37. The second probe corresponds to the 6281-st to 6317-th nucleotides of the ESR1 gene, and the third probe corresponds to the 6385-th to 6419-th nucleotides of the ESR1 gene.

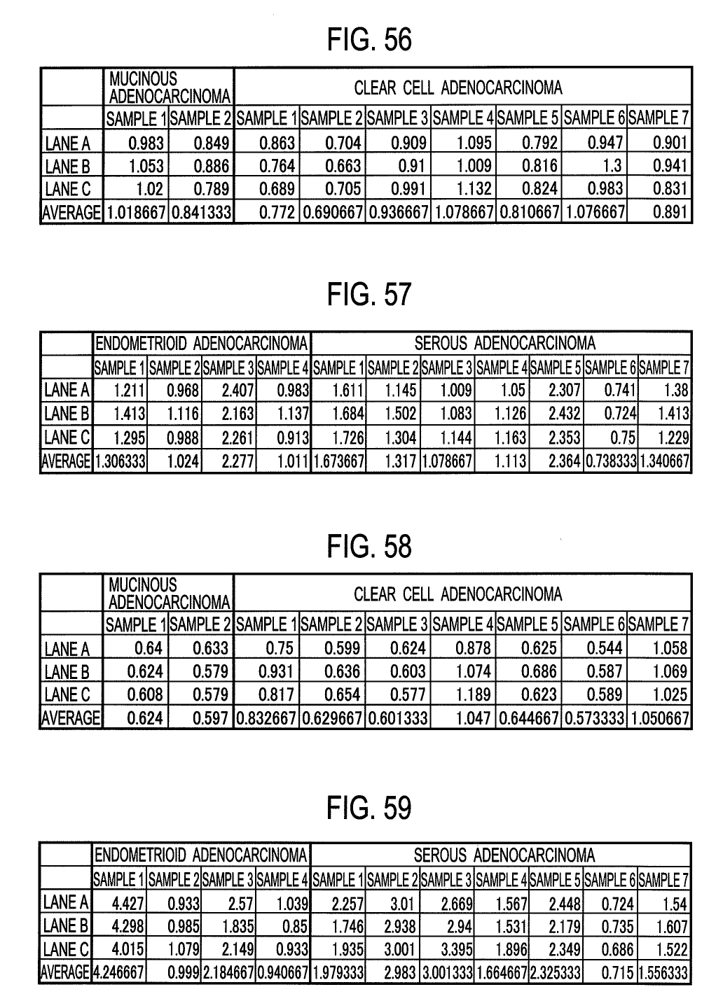

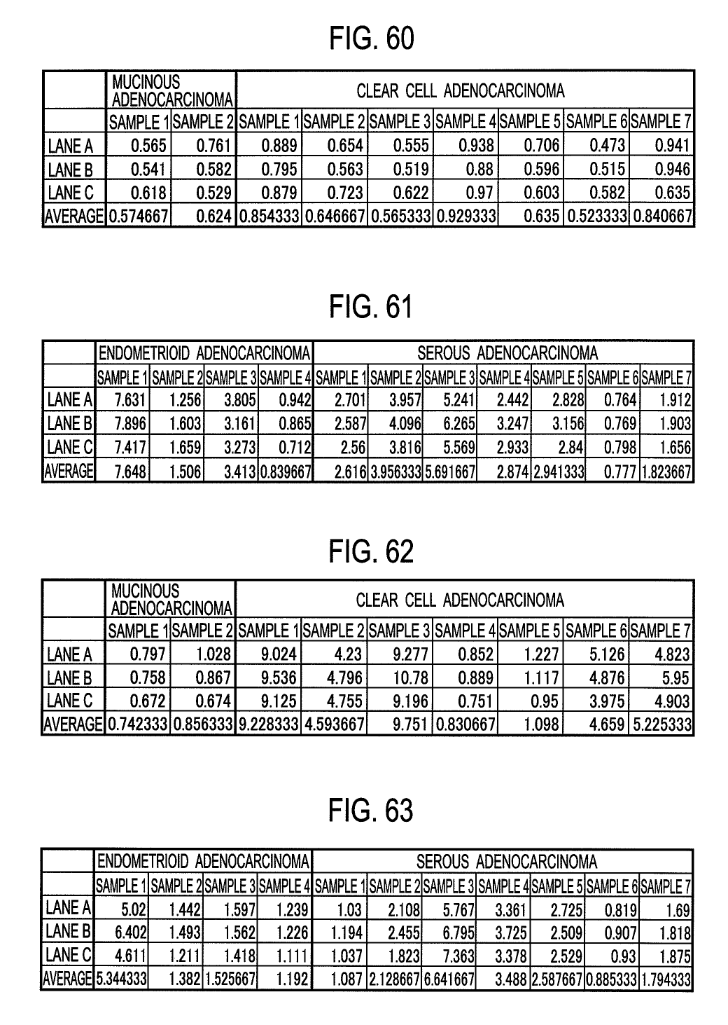

FIG. 56 and FIG. 57 show the detected intensities of the anti-sense RNAs of the ESR1 gene in the case of using the first probe, FIG. 58 and FIG. 59 show the detected intensities concerned in the case of using the second probe, and FIG. 60 and FIG. 61 show the detected intensities concerned in the case of using the third probe. Heretofore, it has been considered that the expression amount of the ESR1 is increased in the ovarian cancer. Moreover, the expression amount of the ESR1 for each of the tissue types has not been reported. As opposed to this, it was shown in the Example that, in the case of using the second probe and the third probe, the detected intensities of the anti-sense RNAs in the ESR1 genes derived from the mucinous adenocarcinoma and the clear cell adenocarcinoma became lower than that in the normal ovarian tissue. Hence, when the amount of such examination-subject RNA to be hybridized with the second probe and the third probe is small, it is possible to diagnose that the tissue from which the examination-subject RNA is derived is the mucinous adenocarcinoma and the clear cell adenocarcinoma.

(Assay of HOST2 Gene Expression)

A first probe for detecting the anti-sense RNA of the HOST2 gene is composed of the base sequence of the sequence number 42, a second probe for the above-described purpose is composed of the base sequence of the sequence number 43, and a third probe for the above-described purpose is composed of the base sequence of the sequence number 44. The first probe corresponds to the 374-th to 408-th nucleotides of the HOST2 gene composed of the base sequence of the sequence number 41. The second probe corresponds to the 465-th to 499-th nucleotides of the HOST2 gene, and the third probe corresponds to the 608-th to 644-th nucleotides of the HOST2 gene.

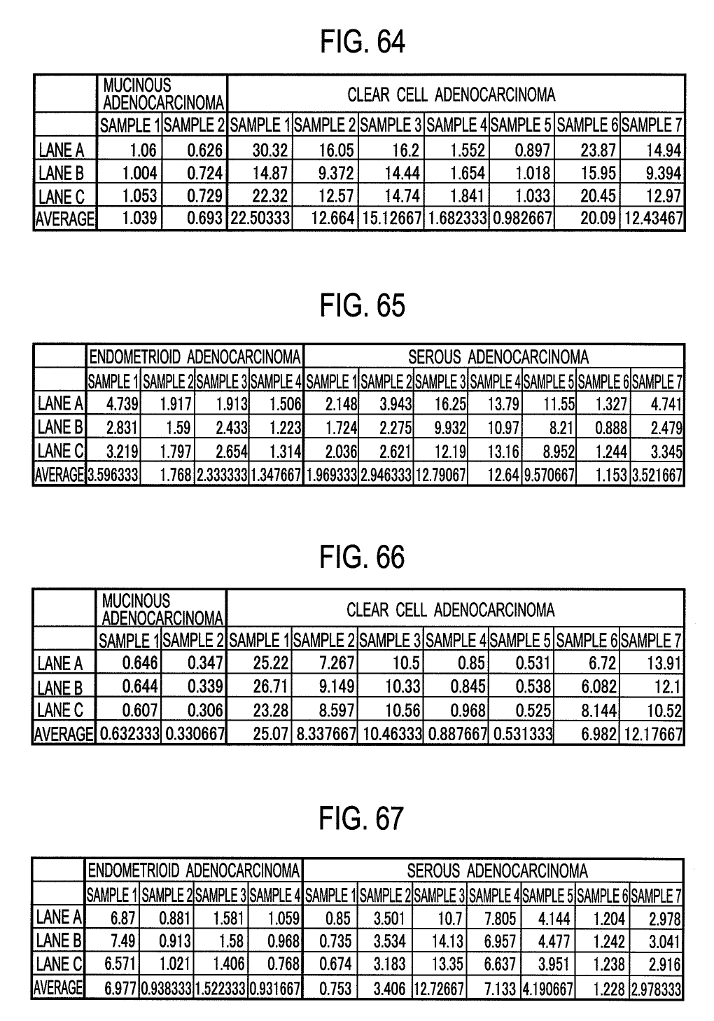

FIG. 62 and FIG. 63 show the detected intensities of the anti-sense RNAs of the HOST2 gene in the case of using the first probe, FIG. 64 and FIG. 65 show the detected intensities concerned in the case of using the second probe, and FIG. 66 and FIG. 67 show the detected intensities concerned in the case of using the third probe. Heretofore, the expression amount of the HOST2 in the ovarian cancer of the Japanese people has not been reported. As opposed to this, it was shown in the Example that, in the Japanese clear cell adenocarcinoma, the Japanese endometrioid adenocarcinoma and the Japanese serous adenocarcinoma, the expression amounts of the HOST2 gene became higher than that in the normal ovarian tissue. Hence, when such an amount of the HOST2 gene is high, it is possible to diagnose that the tissue concerned is the clear cell adenocarcinoma, the endometrioid adenocarcinoma or the serous adenocarcinoma.

(Assay of HSD17B1 Gene Expression)

A first probe for detecting the anti-sense RNA of the HSD17B1 gene is composed of the base sequence of the sequence number 46, a second probe for the above-described purpose is composed of the base sequence of the sequence number 47, and a third probe for the above-described purpose is composed of the base sequence of the sequence number 48. The first probe corresponds to the 1305-th to 1340-th nucleotides of the HSD17B1 gene composed of the base sequence of the sequence number 45. The second probe corresponds to the 1445-th to 1479-th nucleotides of the HSD17B1 gene, and the third probe corresponds to the 2056-th to 2090-th nucleotides of the HSD17B1 gene.

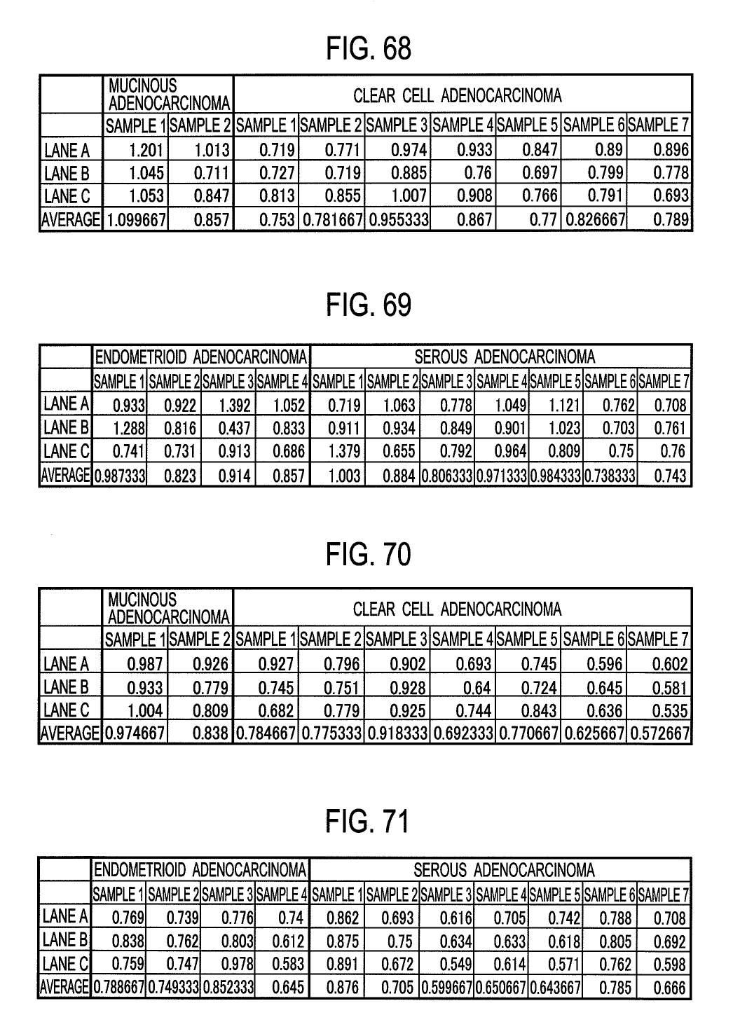

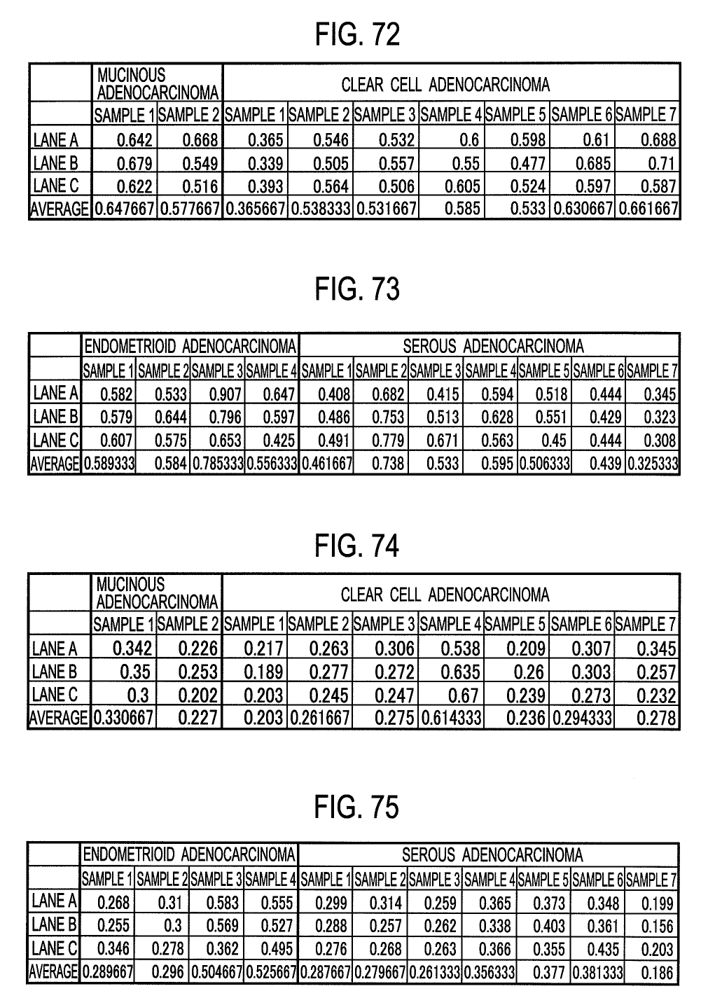

FIG. 68 and FIG. 69 show the detected intensities of the anti-sense RNAs of the HSD17B1 gene in the case of using the first probe, FIG. 70 and FIG. 71 show the detected intensities concerned in the case of using the second probe, and FIG. 72 and FIG. 73 show the detected intensities concerned in the case of using the third probe. Heretofore, the large amount of expression of the HSD17B1 in the normal ovarian tissue has been reported. However, it was shown in the Example that, in the case of using the third probe, the detected intensities of the anti-sense RNAs in the HSD17B1 genes derived from the clear cell adenocarcinoma and the serous adenocarcinoma became lower than that in the normal ovarian tissue. Hence, when the amount of such examination-subject RNA to be hybridized with the third probe is small, it is possible to diagnose that the tissue from which the examination-subject RNA is derived is the clear cell adenocarcinoma or the serous adenocarcinoma.

(Assay of IGFBP4 Gene Expression)

A first probe for detecting the anti-sense RNA of the IGFBP4 gene is composed of the base sequence of the sequence number 50, a second probe for the above-described purpose is composed of the base sequence of the sequence number 51, and a third probe for the above-described purpose is composed of the base sequence of the sequence number 52. The first probe corresponds to the 1100-th to 1134-th nucleotides of the IGFBP4 gene composed of the base sequence of the sequence number 49. The second probe corresponds to the 1360-th to 1394-th nucleotides of the IGFBP4 gene, and the third probe corresponds to the 1772-nd to 1806-th nucleotides of the IGFBP4 gene.

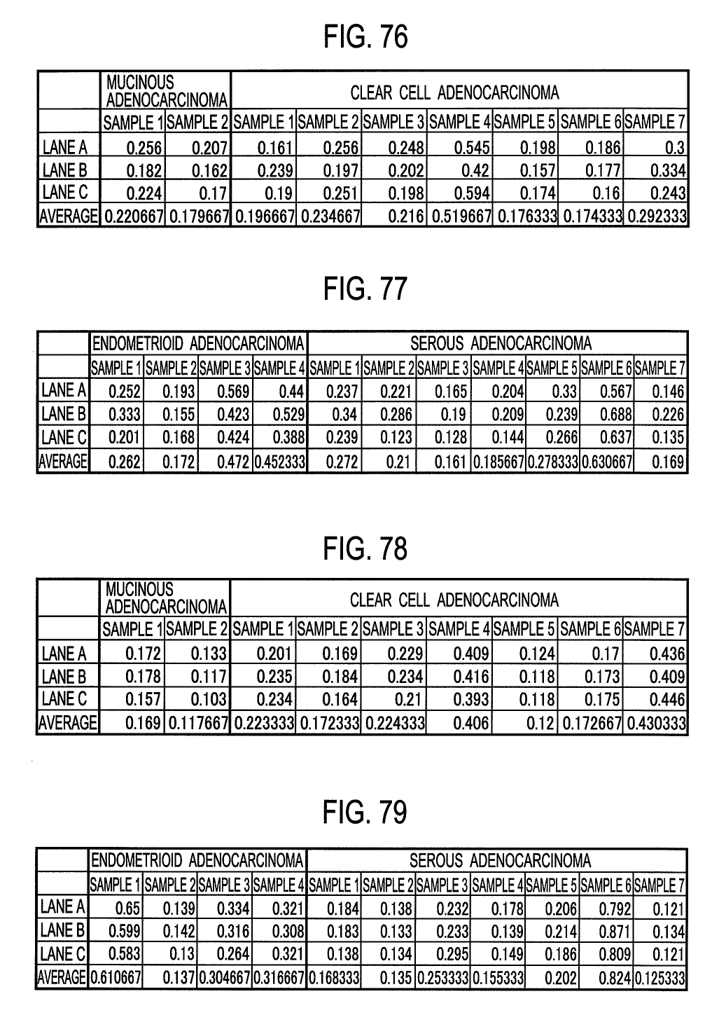

FIG. 74 and FIG. 75 show the detected intensities of the anti-sense RNAs of the IGFBP4 gene in the case of using the first probe, FIG. 76 and FIG. 77 show the detected intensities concerned in the case of using the second probe, and FIG. 78 and FIG. 79 show the detected intensities concerned in the case of using the third probe. Heretofore, it has been considered that the expression amount of the IGFBP4 is increased in the ovarian cancer. Moreover, the expression amount of the IGFBP4 for each of the tissue types has not been reported. As opposed to this, it was shown in the Example that, in all of the tissue types of the mucinous adenocarcinoma, the clear cell adenocarcinoma, the endometrioid adenocarcinoma and the serous adenocarcinoma, the expression amounts of the IGFBP4 gene became lower than that in the normal ovarian tissue. Hence, when such an amount of the IGFBP4 gene is low, it is possible to diagnose that the tissue concerned is the epithelial ovarian carcinoma.

(Assay of IGFBP6 Gene Expression)

A first probe for detecting the anti-sense RNA of the IGFBP6 gene is composed of the base sequence of the sequence number 54, a second probe for the above-described purpose is composed of the base sequence of the sequence number 55, and a third probe for the above-described purpose is composed of the base sequence of the sequence number 56. The first probe corresponds to the 445-th to 479-th nucleotides of the IGFBP6 gene composed of the base sequence of the sequence number 53. The second probe corresponds to the 529-th to 563-rd nucleotides of the IGFBP6 gene, and the third probe corresponds to the 609-th to 643-rd nucleotides of the IGFBP6 gene.

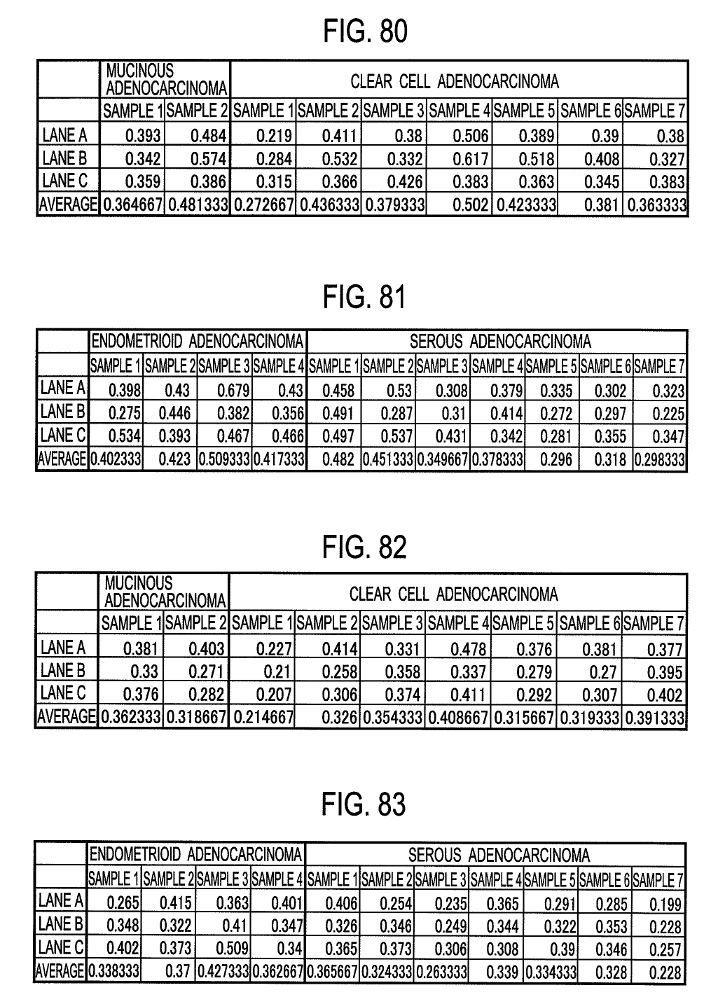

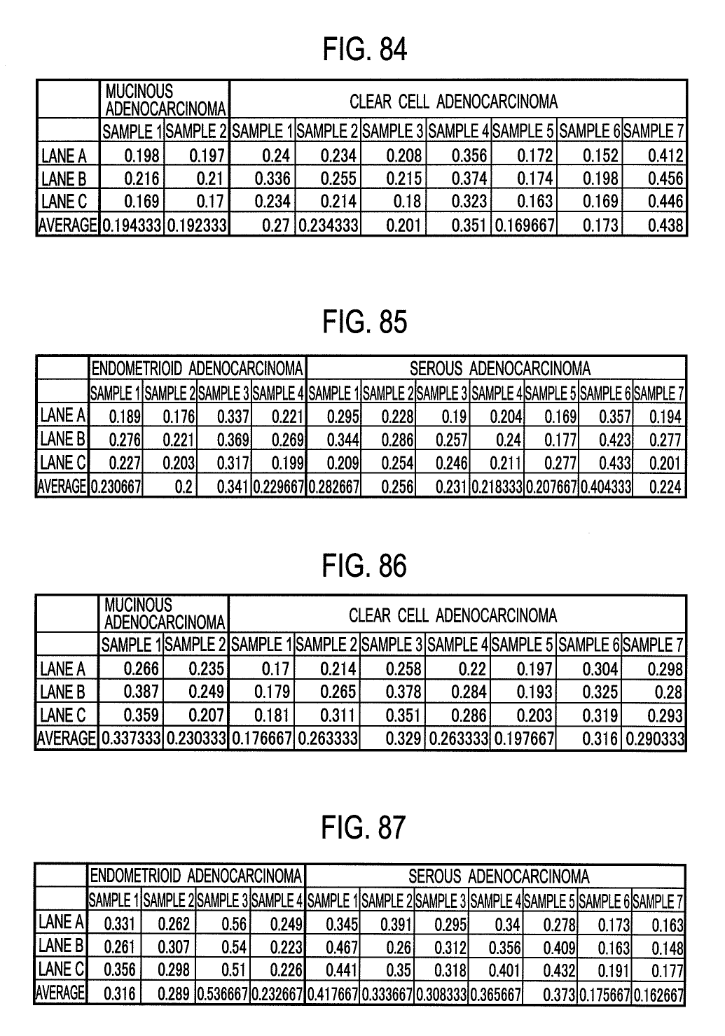

FIG. 80 and FIG. 81 show the detected intensities of the anti-sense RNAs of the IGFBP6 gene in the case of using the first probe, FIG. 82 and FIG. 83 show the detected intensities concerned in the case of using the second probe, and FIG. 84 and FIG. 85 show the detected intensities concerned in the case of using the third probe. Heretofore, the expression amount of the IGFBP6 in the ovarian cancer has not been reported. As opposed to this, it was shown in the Example that, in all of the tissue types of the mucinous adenocarcinoma, the clear cell adenocarcinoma, the endometrioid adenocarcinoma and the serous adenocarcinoma, the expression amounts of the IGFBP6 became lower than that in the normal ovarian tissue. Hence, when such an amount of the IGFBP6 gene is low, it is possible to diagnose that the tissue concerned is the epithelial ovarian carcinoma.

(Assay of INHA Gene Expression)

A first probe for detecting the anti-sense RNA of the INHA gene is composed of the base sequence of the sequence number 58, a second probe for the above-described purpose is composed of the base sequence of the sequence number 59, and a third probe for the above-described purpose is composed of the base sequence of the sequence number 60. The first probe corresponds to the 374-th to 408-th nucleotides of the INHA gene composed of the base sequence of the sequence number 57. The second probe corresponds to the 465-th to 499-th nucleotides of the INHA gene, and the third probe corresponds to the 608-th to 644-th nucleotides of the INHA gene.

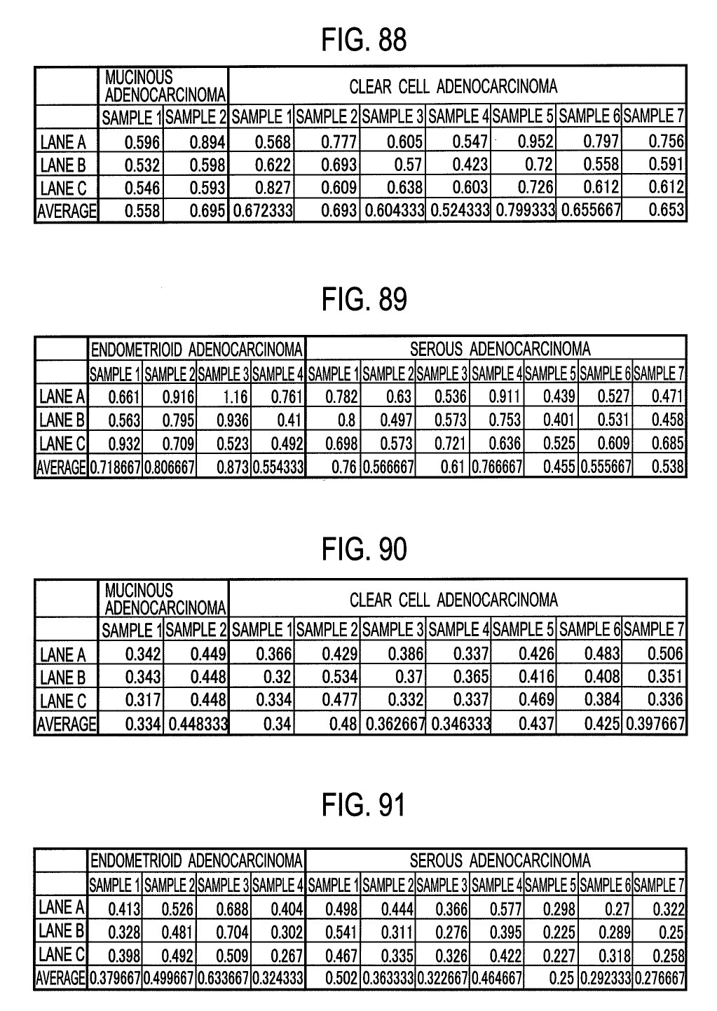

FIG. 86 and FIG. 87 show the detected intensities of the anti-sense RNAs of the INHA gene in the case of using the first probe, FIG. 88 and FIG. 89 show the detected intensities concerned in the case of using the second probe, and FIG. 90 and FIG. 91 show the detected intensities concerned in the case of using the third probe. Heretofore, it has been considered that the expression amount of the INHA in the ovarian cancer is high. Moreover, the expression amount of the INHA for each of the tissue types has not been reported. As opposed to this, it was shown in the Example that, in all of the tissue types of the mucinous adenocarcinoma, the clear cell adenocarcinoma, the endometrioid adenocarcinoma and the serous adenocarcinoma, the expression amounts of the INHA became lower than that in the normal ovarian tissue. In particular, the use of the first probe and the third probe gave remarkable results. Hence, when such an amount of the INHA gene is low, it is possible to diagnose that the tissue concerned is the epithelial ovarian carcinoma.

(Assay of KRT7_{—}1 Gene Expression)

A first probe for detecting the anti-sense RNA of the KRT7_{—}1 gene is composed of the base sequence of the sequence number 62, a second probe for the above-described purpose is composed of the base sequence of the sequence number 63, and a third probe for the above-described purpose is composed of the base sequence of the sequence number 64. The first probe corresponds to the 797-th to 832-nd nucleotides of the KRT7_{—}1 gene composed of the base sequence of the sequence number 61. The second probe corresponds to the 1092-nd to 1126-th nucleotides of the KRT7_{—}1 gene, and the third probe corresponds to the 1610-th to 1644-th nucleotides of the KRT7_{—}1 gene.

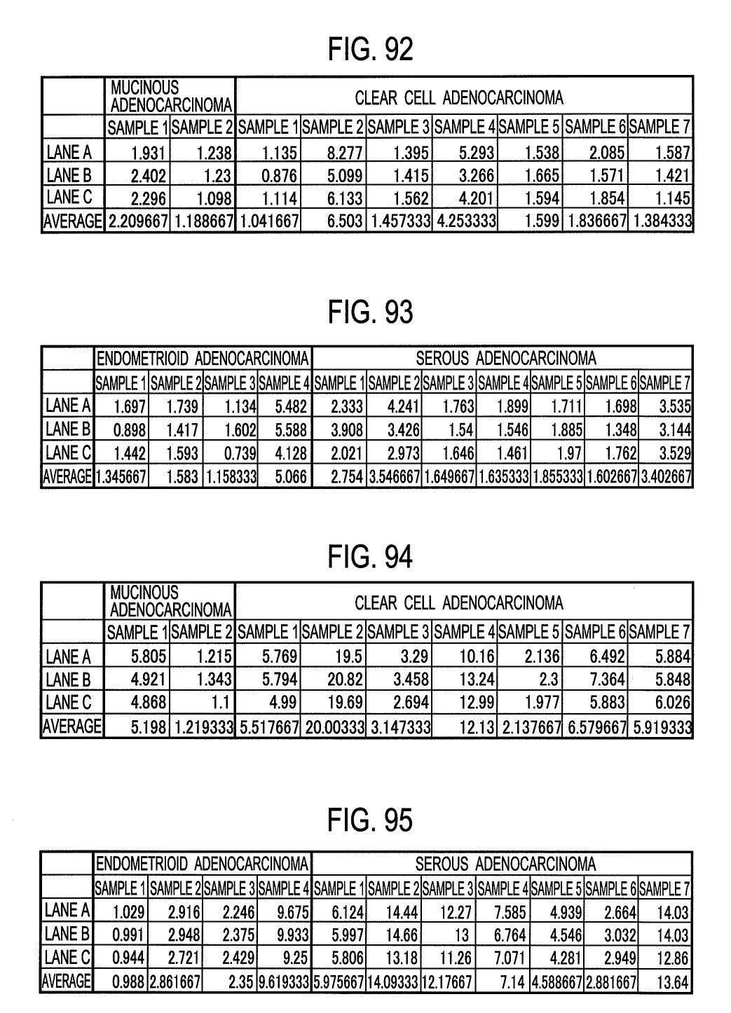

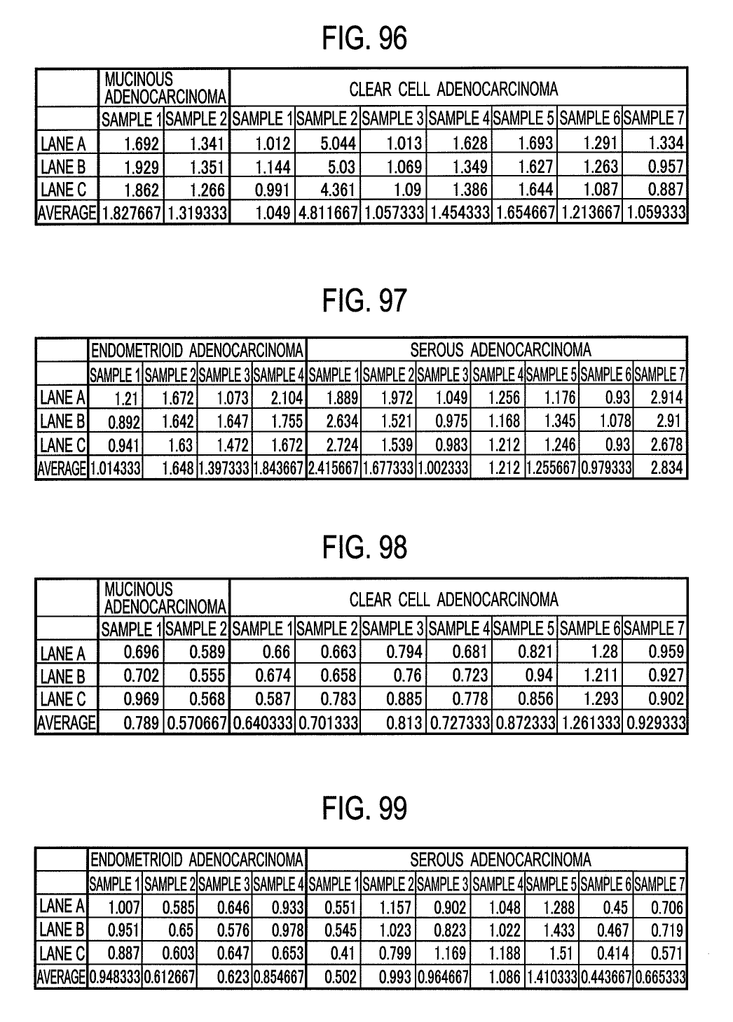

FIG. 92 and FIG. 93 show the detected intensities of the anti-sense RNAs of the KRT7_{—}1 gene in the case of using the first probe, FIG. 94 and FIG. 95 show the detected intensities concerned in the case of using the second probe, and FIG. 96 and FIG. 97 show the detected intensities concerned in the case of using the third probe. Heretofore, the expression amount of the KRT7_{—}1 in the clear cell adenocarcinoma has not been reported. As opposed to this, it was shown in the Example that the expression amounts of the KRT7_{—}1 in the clear cell adenocarcinoma became higher than that in the normal ovarian tissue. Hence, when such an amount of the KRT7_{—}1 gene is high, it is possible to diagnose that the tissue concerned is the epithelial ovarian carcinoma.

(Assay of LAMA2 Gene Expression)

A first probe for detecting the anti-sense RNA of the LAMA2 gene is composed of the base sequence of the sequence number 66, a second probe for the above-described purpose is composed of the base sequence of the sequence number 67, and a third probe for the above-described purpose is composed of the base sequence of the sequence number 68. The first probe corresponds to the 8695-th to 8729-th nucleotides of the LAMA2 gene composed of the base sequence of the sequence number 65. The second probe corresponds to the 8759-th to 8793-rd nucleotides of the LAMA2 gene, and the third probe corresponds to the 9271-st to 9305-th nucleotides of the LAMA2 gene.

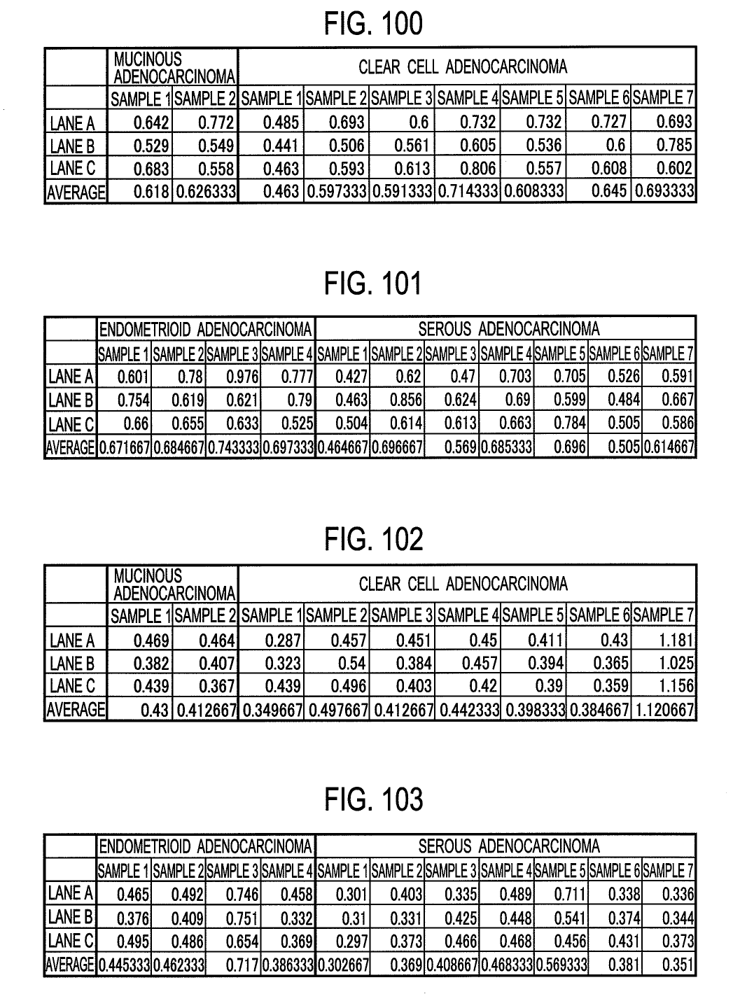

FIG. 98 and FIG. 99 show the detected intensities of the anti-sense RNAs of the LAMA2 gene in the case of using the first probe, FIG. 100 and FIG. 101 show the detected intensities concerned in the case of using the second probe, and FIG. 102 and FIG. 103 show the detected intensities concerned in the case of using the third probe. Heretofore, it has been considered that the expression amount of the LAMA2 is increased in the ovarian cancer. Moreover, the expression amount of the LAMA2 for each of the tissue types has not been reported. As opposed to this, it was shown in the Example that, in all of the tissue types of the mucinous adenocarcinoma, the clear cell adenocarcinoma, the endometrioid adenocarcinoma and the serous adenocarcinoma, the expression amounts of the LAMA2 became lower than that in the normal ovarian tissue. In particular, the use of the third probe gave remarkable results. Hence, when such an amount of the LAMA2 gene is low, it is possible to diagnose that the tissue concerned is the epithelial ovarian carcinoma.

(Assay of MMP2 Gene Expression)

A first probe for detecting the anti-sense RNA of the MMP2 gene is composed of the base sequence of the sequence number 70, a second probe for the above-described purpose is composed of the base sequence of the sequence number 71, and a third probe for the above-described purpose is composed of the base sequence of the sequence number 72. The first probe corresponds to the 2635-th to 2699-th nucleotides of the MMP2 gene composed of the base sequence of the sequence number 69. The second probe corresponds to the 2862-nd to 2896-th nucleotides of the MMP2 gene, and the third probe corresponds to the 3439-th to 3473-rd nucleotides of the MMP2 gene.

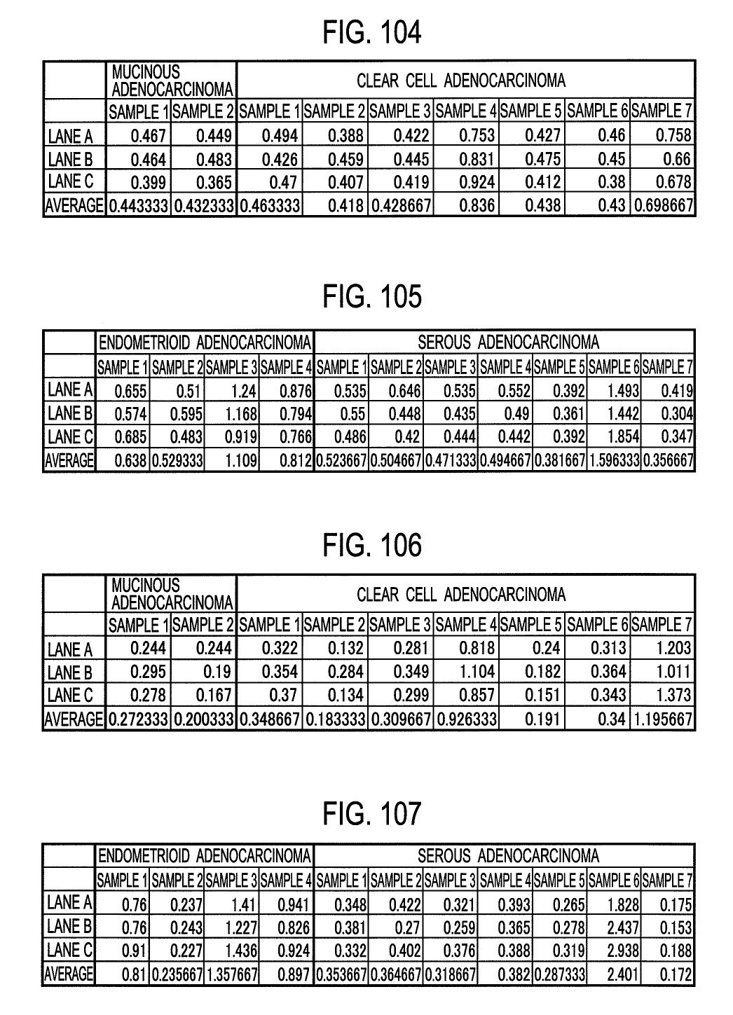

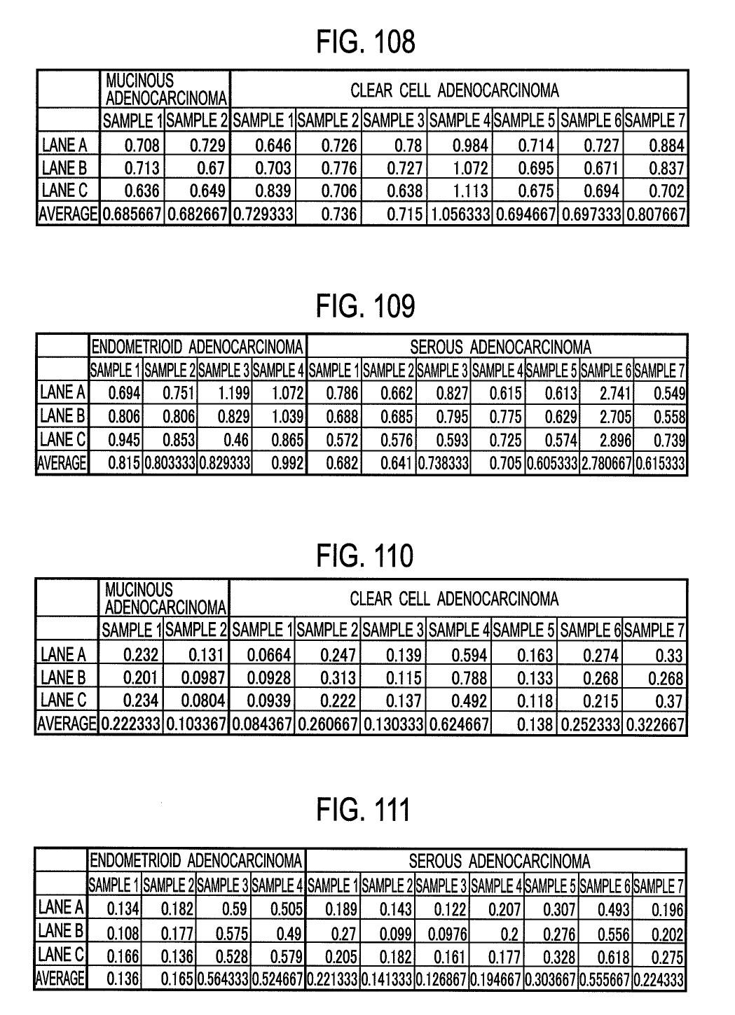

FIG. 104 and FIG. 105 show the detected intensities of the anti-sense RNAs of the MMP2 gene in the case of using the first probe, FIG. 106 and FIG. 107 show the detected intensities concerned in the case of using the second probe, and FIG. 108 and FIG. 109 show the detected intensities concerned in the case of using the third probe. Heretofore, it has been considered that the expression amount of the MMP2 is increased in the ovarian cancer. Moreover, the expression amount of the LAMA2 for each of the tissue types has not been reported. As opposed to this, it was shown in the Example that, in the mucinous adenocarcinoma, the clear cell adenocarcinoma and the serous adenocarcinoma, the expression amounts of the MMP2 became lower than that in the normal ovarian tissue. Hence, when such an amount of the MMP2 gene is low, it is possible to diagnose that the tissue concerned is the mucinous adenocarcinoma, the clear cell adenocarcinoma or the serous adenocarcinoma.

(Assay of TIMP1_{—}1 Gene Expression)

A first probe for detecting the anti-sense RNA of the TIMP1_{—}1 gene is composed of the base sequence of the sequence number 74, a second probe for the above-described purpose is composed of the base sequence of the sequence number 75, and a third probe for the above-described purpose is composed of the base sequence of the sequence number 76. The first probe corresponds to the 293-rd to 327-th nucleotides of the TIMP1_{—}1 gene composed of the base sequence of the sequence number 73. The second probe corresponds to the 345-th to 379-th nucleotides of the TIMP1_{—}1 gene, and the third probe corresponds to the 451-st to 491-st nucleotides of the TIMP1_{—}1 gene.

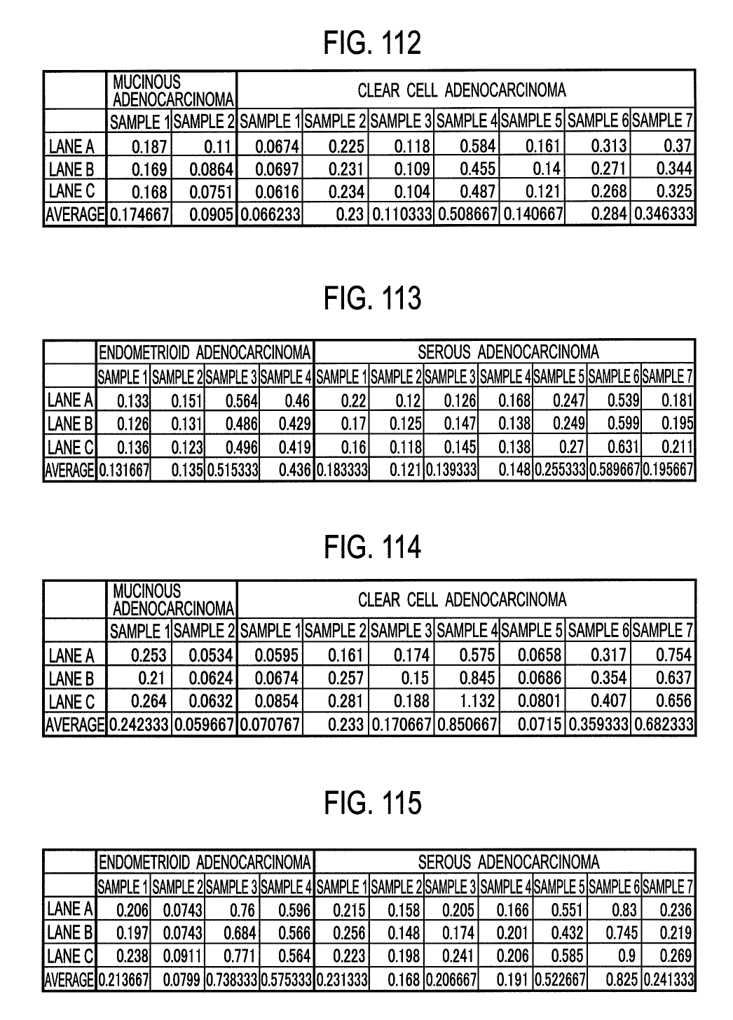

FIG. 110 and FIG. 111 show the detected intensities of the anti-sense RNAs of the TIMP1_{—}1 gene in the case of using the first probe, FIG. 112 and FIG. 113 show the detected intensities concerned in the case of using the second probe, and FIG. 114 and FIG. 115 show the detected intensities concerned in the case of using the third probe. Heretofore, it has been considered that the expression amount of the TIMP1_{—}1 is increased in the ovarian cancer. Moreover, the expression amount of the TIMP1_{—}1 for each of the tissue types has not been reported. As opposed to this, it was shown in the Example that, in all of the tissue types of the mucinous adenocarcinoma, the clear cell adenocarcinoma, the endometrioid adenocarcinoma and the serous adenocarcinoma, the expression amounts of the TIMP1_{—}1 became lower than that in the normal ovarian tissue. Hence, when such an amount of the TIMP1_{—}1 gene is low, it is possible to diagnose that the tissue concerned is the epithelial ovarian carcinoma.

### SEQUENCE LIST

Sequence numbers 1 to 76 described in a sequence table of this specification denote the following sequences.

[Sequence number: 1] Base sequence of ApoA1_{—}1 gene
[Sequence number: 2] Base sequence of first probe of anti-sense RNA of ApoA1_{—}1 gene
[Sequence number: 3] Base sequence of second probe of anti-sense RNA of ApoA1_{—}1 gene
[Sequence number: 4] Base sequence of third probe of anti-sense RNA of ApoA1_{—}1 gene
[Sequence number: 5] Base sequence of ApoE_{—}2 gene
[Sequence number: 6] Base sequence of first probe of anti-sense RNA of ApoE_{—}2 gene
[Sequence number: 7] Base sequence of second probe of anti-sense RNA of ApoE_{—}2 gene
[Sequence number: 8] Base sequence of third probe of anti-sense RNA of ApoE_{—}2 gene
[Sequence number: 9] Base sequence of ApoJ_{—}1 gene
[Sequence number: 10] Base sequence of first probe of anti-sense RNA of ApoJ_{—}1 gene
[Sequence number: 11] Base sequence of second probe of anti-sense RNA of ApoJ_{—}1 gene
[Sequence number: 12] Base sequence of third probe of anti-sense RNA of ApoJ_{—}1 gene
[Sequence number: 13] Base sequence of ARL-1_{—}1 gene
[Sequence number: 14] Base sequence of first probe of anti-sense RNA of ARL-1_{—}1 gene
[Sequence number: 15] Base sequence of second probe of anti-sense RNA of ARL-1_{—}1 gene
[Sequence number: 16] Base sequence of third probe of anti-sense RNA of ARL-1_{—}1 gene
[Sequence number: 17] Base sequence of BST2_{—}1 gene
[Sequence number: 18] Base sequence of first probe of anti-sense RNA of BST2_{—}1 gene
[Sequence number: 19] Base sequence of second probe of anti-sense RNA of BST2_{—}1 gene
[Sequence number: 20] Base sequence of third probe of anti-sense RNA of BST2_{—}1 gene
[Sequence number: 21] Base sequence of CCNE1_{—}1 gene
[Sequence number: 22] Base sequence of first probe of anti-sense RNA of CCNE1_{—}1 gene
[Sequence number: 23] Base sequence of second probe of anti-sense RNA of CCNE1_{—}1 gene
[Sequence number: 24] Base sequence of third probe of anti-sense RNA of CCNE1_{—}1 gene
[Sequence number: 25] Base sequence of CDK4 gene
[Sequence number: 26] Base sequence of first probe of anti-sense RNA of CDK4 gene
[Sequence number: 27] Base sequence of second probe of anti-sense RNA of CDK4 gene
[Sequence number: 28] Base sequence of third probe of anti-sense RNA of CDK4 gene
[Sequence number: 29] Base sequence of CTNNB1 gene
[Sequence number: 30] Base sequence of first probe of anti-sense RNA of CTNNB1 gene
[Sequence number: 31] Base sequence of second probe of anti-sense RNA of CTNNB1 gene
[Sequence number: 32] Base sequence of third probe of anti-sense RNA of CTNNB1 gene
[Sequence number: 33] Base sequence of ERBB2_{—}1 gene
[Sequence number: 34] Base sequence of first probe of anti-sense RNA of ERBB2_{—}1 gene
[Sequence number: 35] Base sequence of second probe of anti-sense RNA of ERBB2_{—}1 gene
[Sequence number: 36] Base sequence of third probe of anti-sense RNA of ERBB2_{—}1 gene
[Sequence number: 37] Base sequence of ESR1 gene
[Sequence number: 38] Base sequence of first probe of anti-sense RNA of ESR1 gene
[Sequence number: 39] Base sequence of second probe of anti-sense RNA of ESR1 gene
[Sequence number: 40] Base sequence of third probe of anti-sense RNA of ESR1 gene
[Sequence number: 41] Base sequence of HOST2 gene
[Sequence number: 42] Base sequence of first probe of anti-sense RNA of HOST2 gene
[Sequence number: 43] Base sequence of second probe of anti-sense RNA of HOST2 gene
[Sequence number: 44] Base sequence of third probe of anti-sense RNA of HOST2 gene
[Sequence number: 45] Base sequence of HSD17B1 gene
[Sequence number: 46] Base sequence of first probe of anti-sense RNA of HSD17B1 gene
[Sequence number: 47] Base sequence of second probe of anti-sense RNA of HSD17B1 gene
[Sequence number: 48] Base sequence of third probe of anti-sense RNA of HSD17B1 gene
[Sequence number: 49] Base sequence of IGFBP4 gene
[Sequence number: 50] Base sequence of first probe of anti-sense RNA of IGFBP4 gene
[Sequence number: 51] Base sequence of second probe of anti-sense RNA of IGFBP4 gene
[Sequence number: 52] Base sequence of third probe of anti-sense RNA of IGFBP4 gene
[Sequence number: 53] Base sequence of IGFBP6 gene
[Sequence number: 54] Base sequence of first probe of anti-sense RNA of IGFBP6 gene
[Sequence number: 55] Base sequence of second probe of anti-sense RNA of IGFBP6 gene
[Sequence number: 56] Base sequence of third probe of anti-sense RNA of IGFBP6 gene
[Sequence number: 57] Base sequence of INHA gene
[Sequence number: 58] Base sequence of first probe of anti-sense RNA of INHA gene
[Sequence number: 59] Base sequence of second probe of anti-sense RNA of INHA gene
[Sequence number: 60] Base sequence of third probe of anti-sense RNA of INHA gene
[Sequence number: 61] Base sequence of KRT7_{—}1 gene
[Sequence number: 62] Base sequence of first probe of anti-sense RNA of KRT7_{—}1 gene
[Sequence number: 63] Base sequence of second probe of anti-sense RNA of KRT7_{—}1 gene
[Sequence number: 64] Base sequence of third probe of anti-sense RNA of KRT7_{—}1 gene
[Sequence number: 65] Base sequence of LAMA2 gene
[Sequence number: 66] Base sequence of first probe of anti-sense RNA of LAMA2 gene
[Sequence number: 67] Base sequence of second probe of anti-sense RNA of LAMA2 gene
[Sequence number: 68] Base sequence of third probe of anti-sense RNA of LAMA2 gene
[Sequence number: 69] Base sequence of MMP2 gene
[Sequence number: 70] Base sequence of first probe of anti-sense RNA of MMP2 gene
[Sequence number: 71] Base sequence of second probe of anti-sense RNA of MMP2 gene
[Sequence number: 72] Base sequence of third probe of anti-sense RNA of MMP2 gene
[Sequence number: 73] Base sequence of TIMP1_{—}1 gene
[Sequence number: 74] Base sequence of first probe of anti-sense RNA of TIMP1_{—}1 gene
[Sequence number: 75] Base sequence of second probe of anti-sense RNA of TIMP1_{—}1 gene
[Sequence number: 76] Base sequence of third probe of anti-sense RNA of TIMP1_{—}1 gene

In the case of displaying the bases by abbreviations in this specification, abbreviations by IUPAC-IUB Commission on Biochemical Nomenclature, or idiomatic abbreviations in the field concerned are used. Examples of the abbreviations are shown below.

a: adenine, t: thymine, g: guanine, c: cytosine, u: uracil

What is claimed is:

1. A molecular diagnosis system of ovarian cancers comprising:
a detection device configured to obtain a detected value of an expression amount of an apolipoprotein A1 gene in ovarian tissue as a diagnosis subject;
a storage device configured to store a normal value of the expression amount of the apolipoprotein A1 gene in normal ovarian tissue; and
a determination mechanism configured to determine that the ovarian tissue as the diagnosis subject is clear cell adenocarcinoma when the detected value is lower than the normal value.

2. A molecular diagnosis system of ovarian cancers comprising:
a detection device configured to obtain a detected value of an expression amount of an apolipoprotein E gene in ovarian tissue as a diagnosis subject;
a storage device configured to store a normal value of the expression amount of the apolipoprotein E gene in normal ovarian tissue; and
a determination mechanism configured to determine that the ovarian tissue as the diagnosis subject is epithelial ovarian carcinoma when the detected value is lower than the normal value.

3. A molecular diagnosis system of ovarian cancers comprising:
a detection device configured to obtain a detected value of an expression amount of an apolipoprotein J gene in ovarian tissue as a diagnosis subject;
a storage device configured to store a normal value of the expression amount of the apolipoprotein J gene in normal ovarian tissue; and
a determination mechanism configured to determine that the ovarian tissue as the diagnosis subject is mucinous adenocarcinoma or clear cell adenocarcinoma when the detected value is lower than the normal value.

4. A molecular diagnosis system of ovarian cancers comprising:
a detection device configured to obtain a detected value of an expression amount of a homo sapiens aldo-keto reductase family 1 member B10 gene in ovarian tissue as a diagnosis subject;
a storage device configured to store a normal value of the expression amount of the homo sapiens aldo-keto reductase family 1 member B10 gene in normal ovarian tissue; and
a determination mechanism configured to determine that the ovarian tissue as the diagnosis subject is mucinous adenocarcinoma when the detected value is higher than the normal value.

5. A molecular diagnosis system of ovarian cancers comprising:
a detection device configured to obtain a detected value of an expression amount of a bone marrow stromal cell antigen 2 gene in ovarian tissue as a diagnosis subject;
a storage device configured to store a normal value of the expression amount of the bone marrow stromal cell antigen 2 gene in normal ovarian tissue; and
a determination mechanism configured to determine that the ovarian tissue as the diagnosis subject is epithelial ovarian carcinoma when the detected value is lower than the normal value.

6. A molecular diagnosis system of ovarian cancers comprising:
a detection device configured to obtain a detected value of an expression amount of a cyclin E1 gene in ovarian tissue as a diagnosis subject;
a storage device configured to store a normal value of the expression amount of the cyclin E1 gene in normal ovarian tissue; and
a determination mechanism configured to determine that the ovarian tissue as the diagnosis subject is clear cell adenocarcinoma or serous adenocarcinoma when the detected value is higher than the normal value.

7. A molecular diagnosis system of ovarian cancers comprising:
a detection device configured to obtain a detected value of an expression amount of a cyclin-dependent kinase 4 gene in ovarian tissue as a diagnosis subject;
a storage device configured to store a normal value of the expression amount of the cyclin-dependent kinase 4 gene in normal ovarian tissue; and
a determination mechanism configured to determine that the ovarian tissue as the diagnosis subject is epithelial ovarian carcinoma when the detected value is lower than the normal value.

8. A molecular diagnosis system of ovarian cancers comprising:
a detection device configured to obtain a detected value of an expression amount of a catenin, beta-1 gene in ovarian tissue as a diagnosis subject;
a storage device configured to store a normal value of the expression amount of the catenin, beta-1 gene in normal ovarian tissue; and
a determination mechanism configured to determine that the ovarian tissue as the diagnosis subject is epithelial ovarian carcinoma when the detected value is lower than the normal value.

9. A molecular diagnosis system of ovarian cancers comprising:
a detection device configured to obtain a detected value of an expression amount of a V-erb-b2 erythroblastic leukemia viral oncogene homolog 2 gene in ovarian tissue as a diagnosis subject;
a storage device configured to store a normal value of the expression amount of the V-erb-b2 erythroblastic leukemia viral oncogene homolog 2 gene in normal ovarian tissue; and
a determination mechanism configured to determine that the ovarian tissue as the diagnosis subject is clear cell adenocarcinoma or serous adenocarcinoma when the detected value is higher than the normal value.

10. A molecular diagnosis system of ovarian cancers comprising:
a detection device configured to obtain a detected value of an expression amount of an estrogen receptor 1 gene in ovarian tissue as a diagnosis subject;
a storage device configured to store a normal value of the expression amount of the estrogen receptor 1 gene in normal ovarian tissue; and
a determination mechanism configured to determine that the ovarian tissue as the diagnosis subject is mucinous adenocarcinoma or clear cell adenocarcinoma when the detected value is lower than the normal value.

11. A molecular diagnosis system of ovarian cancers comprising:
a detection device configured to obtain a detected value of an expression amount of a human ovarian cancer specific transcript 2 gene in ovarian tissue as a diagnosis subject;
a storage device configured to store a normal value of the expression amount of the human ovarian cancer specific transcript 2 gene in normal ovarian tissue; and
a determination mechanism configured to determine that the ovarian tissue as the diagnosis subject is clear cell adenocarcinoma, endometrioid adenocarcinoma or serous adenocarcinoma when the detected value is higher than the normal value.

12. A molecular diagnosis system of ovarian cancers comprising:
a detection device configured to obtain a detected value of an expression amount of a hydroxysteroid (17-beta) dehydrogenase 1 gene in ovarian tissue as a diagnosis subject;
a storage device configured to store a normal value of the expression amount of the hydroxysteroid (17-beta) dehydrogenase 1 gene in normal ovarian tissue; and
a determination mechanism configured to determine that the ovarian tissue as the diagnosis subject is clear cell adenocarcinoma or serous adenocarcinoma when the detected value is lower than the normal value.

13. A molecular diagnosis system of ovarian cancers comprising:
a detection device configured to obtain a detected value of an expression amount of an insulin-like growth factor binding protein 4 gene in ovarian tissue as a diagnosis subject;
a storage device configured to store a normal value of the expression amount of the insulin-like growth factor binding protein 4 gene in normal ovarian tissue; and
a determination mechanism configured to determine that the ovarian tissue as the diagnosis subject is epithelial ovarian carcinoma when the detected value is lower than the normal value.

14. A molecular diagnosis system of ovarian cancers comprising:
a detection device configured to obtain a detected value of an expression amount of an insulin-like growth factor binding protein 6 gene in ovarian tissue as a diagnosis subject;
a storage device configured to store a normal value of the expression amount of the insulin-like growth factor binding protein 6 gene in normal ovarian tissue; and
a determination mechanism configured to determine that the ovarian tissue as the diagnosis subject is epithelial ovarian carcinoma when the detected value is lower than the normal value.

15. A molecular diagnosis system of ovarian cancers comprising:
a detection device configured to obtain a detected value of an expression amount of an inhibin alpha gene in ovarian tissue as a diagnosis subject;
a storage device configured to store a normal value of the expression amount of the inhibin alpha gene in normal ovarian tissue; and
a determination mechanism configured to determine that the ovarian tissue as the diagnosis subject is epithelial ovarian carcinoma when the detected value is lower than the normal value.

16. A molecular diagnosis system of ovarian cancers comprising:
a detection device configured to obtain a detected value of an expression amount of a keratin 7 gene in ovarian tissue as a diagnosis subject;
a storage device configured to store a normal value of the expression amount of the keratin 7 gene in normal ovarian tissue; and
a determination mechanism configured to determine that the ovarian tissue as the diagnosis subject is epithelial ovarian carcinoma when the detected value is higher than the normal value.

17. A molecular diagnosis system of ovarian cancers comprising:
a detection device configured to obtain a detected value of an expression amount of a laminin, alpha 2 gene in ovarian tissue as a diagnosis subject;
a storage device configured to store a normal value of the expression amount of the laminin, alpha 2 gene in normal ovarian tissue; and
a determination mechanism configured to determine that the ovarian tissue as the diagnosis subject is epithelial ovarian carcinoma when the detected value is lower than the normal value.

18. A molecular diagnosis system of ovarian cancers comprising:
a detection device configured to obtain a detected value of an expression amount of a matrix metallopeptidase 2 gene in ovarian tissue as a diagnosis subject;
a storage device configured to store a normal value of the expression amount of the matrix metallopeptidase 2 gene in normal ovarian tissue; and
a determination mechanism configured to determine that the ovarian tissue as the diagnosis subject is mucinous adenocarcinoma, clear cell adenocarcinoma or serous adenocarcinoma when the detected value is lower than the normal value.

19. A molecular diagnosis system of ovarian cancers comprising:
a detection device configured to obtain a detected value of an expression amount of a tissue inhibitor of metalloproteinase 1 gene in ovarian tissue as a diagnosis subject;
a storage device configured to store a normal value of the expression amount of the tissue inhibitor of metalloproteinase 1 gene in normal ovarian tissue; and
a determination mechanism configured to determine that the ovarian tissue as the diagnosis subject is epithelial ovarian carcinoma when the detected value is lower than the normal value.

20. A molecular diagnosis method of ovarian cancers comprising:
obtaining a detected value of an expression amount of an apolipoprotein A1 gene in ovarian tissue as a diagnosis subject; and
determining that the ovarian tissue as the diagnosis subject is clear cell adenocarcinoma when the detected value is lower than a normal value of the expression amount of the apolipoprotein A1 gene in normal ovarian tissue.

21. A molecular diagnosis method of ovarian cancers comprising:
obtaining a detected value of an expression amount of an apolipoprotein E gene in ovarian tissue as a diagnosis subject; and
determining that the ovarian tissue as the diagnosis subject is epithelial ovarian carcinoma when the detected value is lower than a normal value of the expression amount of the apolipoprotein E gene in normal ovarian tissue.

22. A molecular diagnosis method of ovarian cancers comprising:
obtaining a detected value of an expression amount of an apolipoprotein J gene in ovarian tissue as a diagnosis subject; and
determining that the ovarian tissue as the diagnosis subject is mucinous adenocarcinoma or clear cell adenocarcinoma when the detected value is lower than a normal value of the expression amount of the apolipoprotein J gene in normal ovarian tissue.

23. A molecular diagnosis method of ovarian cancers comprising:
obtaining a detected value of an expression amount of a homo sapiens aldo-keto reductase family 1 member B10 gene in ovarian tissue as a diagnosis subject; and
determining that the ovarian tissue as the diagnosis subject is mucinous adenocarcinoma when the detected value is higher than a normal value of the expression amount of the homo sapiens aldo-keto reductase family 1 member B10 gene in normal ovarian tissue.

24. A molecular diagnosis method of ovarian cancers comprising:
obtaining a detected value of an expression amount of a bone marrow stromal cell antigen 2 gene in ovarian tissue as a diagnosis subject; and
determining that the ovarian tissue as the diagnosis subject is epithelial ovarian carcinoma when the detected value is lower than a normal value of the expression amount of the bone marrow stromal cell antigen 2 gene in normal ovarian tissue.

25. A molecular diagnosis method of ovarian cancers comprising:
obtaining a detected value of an expression amount of a cyclin E1 gene in ovarian tissue as a diagnosis subject; and
determining that the ovarian tissue as the diagnosis subject is clear cell adenocarcinoma or serous adenocarcinoma when the detected value is higher than a normal value of the expression amount of the cyclin E1 gene in normal ovarian tissue.

26. A molecular diagnosis method of ovarian cancers comprising:
obtaining a detected value of an expression amount of a cyclin-dependent kinase 4 gene in ovarian tissue as a diagnosis subject; and
determining that the ovarian tissue as the diagnosis subject is epithelial ovarian carcinoma when the detected value is lower than a normal value of the expression amount of the cyclin-dependent kinase 4 gene in normal ovarian tissue.

27. A molecular diagnosis method of ovarian cancers comprising:
obtaining a detected value of an expression amount of a catenin, beta-1 gene in ovarian tissue as a diagnosis subject; and
determining that the ovarian tissue as the diagnosis subject is epithelial ovarian carcinoma when the detected value is lower than a normal value of the expression amount of the catenin, beta-1 gene in normal ovarian tissue.

28. A molecular diagnosis method of ovarian cancers comprising:
obtaining a detected value of an expression amount of a V-erb-b2 erythroblastic leukemia viral oncogene homolog 2 gene in ovarian tissue as a diagnosis subject; and
determining that the ovarian tissue as the diagnosis subject is clear cell adenocarcinoma or serous adenocarcinoma when the detected value is higher than a normal value of the expression amount of the V-erb-b2 erythroblastic leukemia viral oncogene homolog 2 gene in normal ovarian tissue.

29. A molecular diagnosis method of ovarian cancers comprising:
obtaining a detected value of an expression amount of an estrogen receptor 1 gene in ovarian tissue as a diagnosis subject; and
determining that the ovarian tissue as the diagnosis subject is mucinous adenocarcinoma or clear cell adenocarcinoma when the detected value is lower than a normal value of the expression amount of the estrogen receptor 1 gene in normal ovarian tissue.

30. A molecular diagnosis method of ovarian cancers comprising:
obtaining a detected value of an expression amount of a human ovarian cancer specific transcript 2 gene in ovarian tissue as a diagnosis subject; and
determining that the ovarian tissue as the diagnosis subject is clear cell adenocarcinoma, endometrioid adenocarcinoma or serous adenocarcinoma when the detected value is higher than a normal value of the expression amount of the human ovarian cancer specific transcript 2 gene in normal ovarian tissue.

31. A molecular diagnosis method of ovarian cancers comprising:
obtaining a detected value of an expression amount of a hydroxysteroid (17-beta) dehydrogenase 1 gene in ovarian tissue as a diagnosis subject; and
determining that the ovarian tissue as the diagnosis subject is clear cell adenocarcinoma or serous adenocarcinoma when the detected value is lower than a normal value of the expression amount of the hydroxysteroid (17-beta) dehydrogenase 1 gene in normal ovarian tissue.

32. A molecular diagnosis method of ovarian cancers comprising:
obtaining a detected value of an expression amount of an insulin-like growth factor binding protein 4 gene in ovarian tissue as a diagnosis subject; and
determining that the ovarian tissue as the diagnosis subject is epithelial ovarian carcinoma when the detected value is lower than a normal value of the expression amount of the insulin-like growth factor binding protein 4 gene in normal ovarian tissue.

33. A molecular diagnosis method of ovarian cancers comprising:
obtaining a detected value of an expression amount of an insulin-like growth factor binding protein 6 gene in ovarian tissue as a diagnosis subject; and
determining that the ovarian tissue as the diagnosis subject is epithelial ovarian carcinoma when the detected value is lower than a normal value of the expression amount of the insulin-like growth factor binding protein 6 gene in normal ovarian tissue.

34. A molecular diagnosis method of ovarian cancers comprising:
obtaining a detected value of an expression amount of an inhibin alpha gene in ovarian tissue as a diagnosis subject; and
determining that the ovarian tissue as the diagnosis subject is epithelial ovarian carcinoma when the detected value is lower than a normal value of the expression amount of the inhibin alpha gene in normal ovarian tissue.

35. A molecular diagnosis method of ovarian cancers comprising:
obtaining a detected value of an expression amount of a keratin 7 gene in ovarian tissue as a diagnosis subject; and
determining that the ovarian tissue as the diagnosis subject is epithelial ovarian carcinoma when the detected value is higher than a normal value of the expression amount of the keratin 7 gene in normal ovarian tissue.

36. A molecular diagnosis method of ovarian cancers comprising:
obtaining a detected value of an expression amount of a laminin, alpha 2 gene in ovarian tissue as a diagnosis subject; and
determining that the ovarian tissue as the diagnosis subject is epithelial ovarian carcinoma when the detected value is lower than a normal value of the expression amount of the laminin, alpha 2 gene in normal ovarian tissue.

37. A molecular diagnosis method of ovarian cancers comprising:
obtaining a detected value of an expression amount of a matrix metallopeptidase 2 gene in ovarian tissue as a diagnosis subject; and
determining that the ovarian tissue as the diagnosis subject is mucinous adenocarcinoma, clear cell adenocarcinoma or serous adenocarcinoma when the detected value is lower than a normal value of the expression amount of the matrix metallopeptidase 2 gene in normal ovarian tissue.

38. A molecular diagnosis method of ovarian cancers comprising:
obtaining a detected value of an expression amount of a tissue inhibitor of metalloproteinase 1 gene in ovarian tissue as a diagnosis subject; and
determining that the ovarian tissue as the diagnosis subject is epithelial ovarian carcinoma when the detected value is lower than a normal value of the expression amount of the tissue inhibitor of metalloproteinase 1 gene in normal ovarian tissue.


SEQUENCE LISTING (76 sequences)

SEQ ID NO: 1 - DNA, 897 bp, Homo sapiens
  Homo sapiens apolipoprotein A-I (APOA1), mRNA.
agagactgcgagaaggaggtcccccacggcccttcaggatgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccagtgaggcgcccgccgccgccccccttcccggtgctcagaataaacgtttccaaagtggg

SEQ ID NO: 2 - DNA, 35 bp, Artificial Sequence
  Synthetic oligonucleotide
cactgtgtacgtggatgtgctcaaagacagcggca

SEQ ID NO: 3 - DNA, 35 bp, Artificial Sequence
  Synthetic oligonucleotide
ctaaacctaaagctccttgacaactgggacagcgt

SEQ ID NO: 4 - DNA, 35 bp, Artificial Sequence
  Synthetic oligonucleotide
ttcctgagcgctctcgaggagtacactaagaagct

SEQ ID NO: 5 - DNA, 1157 bp, Homo sapiens
  Human apolipoprotein E mRNA, complete cds.
ccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaagtcacccaagaactgagggcgctgatggacgagaccatgaaggagttgaaggcctacaaatcggaactggaggaacaactgaccccggtagcggaggagacgcgggcacggctgtccaaggagctgcagacggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatcccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagtcggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgc

SEQ ID NO: 6 - DNA, 35 bp, Artificial Sequence
  Synthetic oligonucleotide
acgagaccatgaaggagttgaaggcctacaaatcg

SEQ ID NO: 7 - DNA, 35 bp, Artificial Sequence
  Synthetic oligonucleotide
tacaaatcggaactggaggaacaactgaccccggt

SEQ ID NO: 8 - DNA, 35 bp, Artificial Sequence
  Synthetic oligonucleotide
cagccgtcctcctggggtggaccctagtttaataa

SEQ ID NO: 9 - DNA, 1512 bp, Homo sapiens
  Human apolipoprotein J mRNA, complete cds.
ttccggtcccccaggacatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccacagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctg

SEQ ID NO: 10 - DNA, 36 bp, Artificial Sequence
  Synthetic oligonucleotide
attcatacgagaaggcgacgatgaccggactgtgtg

SEQ ID NO: 11 - DNA, 35 bp, Artificial Sequence
  Synthetic oligonucleotide
tgaaggaccagtgtgacaagtgccgggagatcttg

SEQ ID NO: 12 - DNA, 35 bp, Artificial Sequence
  Synthetic oligonucleotide
tggcaaacctcacgcaaggcgaagaccagtactat

SEQ ID NO: 13 - DNA, 1551 bp, Homo sapiens
  Homo sapiens aldo-keto reductase family 1, member B10 (aldose reductase)(AKR1B10), mRNA.
cctgagctcaggagtttgagaccagcctgtctctactaacaatataaaaattagctgggagtcacggtgggcgcctgtaatcccagctactcgggaggctgaggcaggagaattgcttgaacccaggagacagaggttgtagtgagctgagatcgcaccactgcactctagccttggcaacagtgcaagactgtctcaaaaacagcaacagagagcaggacgtgagacttctacctgctcactcagaatcatttctgcaccaaccatggccacgtttgtggagctcagtaccaaagccaagatgcccattgtgggcctgggcacttggaagtctcctcttggcaaagtgaaagaagcagtgaaggtggccattgatgcaggatatcggcacattgactgtgcctatgtctatcagaatgaacatgaagtgggggaagccatccaagagaagatccaagagaaggctgtgaagcgggaggacctgttcatcgtcagcaagttgtggcccactttctttgagagaccccttgtgaggaaagcctttgagaagaccctcaaggacctgaagctgagctatctggacgtctatcttattcactggccacagggattcaagtctggggatgaccttttccccaaagatgataaaggtaatgccatcggtggaaaagcaacgttcttggatgcctgggaggccatggaggagctggtggatgaggggctggtgaaagcccttggggtctccaatttcagccacttccagatcgagaagctcttgaacaaacctggactgaaatataaaccagtgactaaccaggttgagtgtcacccatacctcacacaggagaaactgatccagtactgccactccaagggcatcaccgttacggcctacagccccctgggctctccggatagaccttgggccaagccagaagacccttccctgctggaggatcccaagattaaggagattgctgcaaagcacaaaaaaaccgcagcccaggttctgatccgtttccatatccagaggaatgtgattgtcatccccaagtctgtgacaccagcacgcattgttgagaacattcaggtctttgactttaaattgagtgatgaggagatggcaaccatactcagcttcaacagaaactggagggcctgtaacgtgttgcaatcctctcatttggaagactatcccttcgatgcagaatattgaggttgaatctcctggtgagattatacaggagattctctttcttcgctgaagtgtgactacctccactcatgtcccattttagccaagcttatttaagatcacagtgaacttagtcctgttatagacgagaatcgaggtgctgttttagacatttatttctgtatgttcaactaggatcagaatatcacagaaaagcatggcttgaataaggaaatgacaattttttccacttatctgatcagaacaaatgtttattaagcatcagaaactctgccaacactgaggatgtaaagatcaataaaaaaaataataatcataaaaaaaaaaaaaaaaaa

SEQ ID NO: 14 - DNA, 36 bp, Artificial Sequence
  Synthetic oligonucleotide
agctgagctatctggacgtctatcttattcactggc

SEQ ID NO: 15 - DNA, 35 bp, Artificial Sequence
  Synthetic oligonucleotide
accagtgactaaccaggttgagtgtcacccatacc

SEQ ID NO: 16 - DNA, 38 bp, Artificial Sequence
  Synthetic oligonucleotide
gtgaacttagtcctgttatagacgagaatcgaggtgct

SEQ ID NO: 17 - DNA, 996 bp, Homo sapiens
  Homo sapiens mRNA for BST-2, complete cds.
gtggaattcatggcatctacttcgtatgactattgcagagtgcccatggaagacggggataagcgctgtaagcttctgctggggataggaattctggtgctcctgatcatcgtgattctgggggtgcccttgattatcttcaccatcaaggccaacagcgaggcctgccgggacggccttcgggcagtgatggagtgtcgcaatgtcacccatctcctgcaacaagagctgaccgaggcccagaagggctttcaggatgtggaggcccaggccgccacctgcaaccacactgtgatggccctaatggcttccctggatgcagagaaggcccaaggacaaaagaaagtggaggagcttgagggagagatcactacattaaaccataagcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtgagatcccaggaagctggcacatcttggaaggtccgtcctgctcggcttttcgcttgaacattcccttgatctcatcagttctgagcgggtcatggggcaacacggttagcggggagagcacggggtagccggagaagggcctctggagcaggtctggaggggccatggggcagtcctgggtgtggggacacagtcgggttgacccagggctgtctccctccagagcctccctccggacaatgagtcccccctcttgtctcccaccctgagattgggcatggggtgcggtgtggggggcatgtgctgcctgttgttatgggttttttttgcggggggggttgcttttttctggggtctttgagctccaaaaaataaacacttcctttgagggagagcaaaaaaaaaaaaaaaaaaaaaaaaaaaaaaaaaaaagaattccaccaca

SEQ ID NO: 18 - DNA, 35 bp, Artificial Sequence
  Synthetic oligonucleotide
tggcatctacttcgtatgactattgcagagtgccc

SEQ ID NO: 19 - DNA, 35 bp, Artificial Sequence
  Synthetic oligonucleotide
agtgatggagtgtcgcaatgtcacccatctcctgc

SEQ ID NO: 20 - DNA, 35 bp, Artificial Sequence
  Synthetic oligonucleotide
acattaaaccataagcttcaggacgcgtctgcaga

SEQ ID NO: 21 - DNA, 1787 bp, Homo sapiens
  Homo sapiens cyclin E1 (CCNE1), transcript variant 2, mRNA.
gtgctcacccggcccggtgccacccgggtccacagggatgcgaaggagcgggacaccatgaaggaggacggcggcgcggagttctcggctcgctccaggaagaggaaggcaaacgtgaccgtttttttgcaggatccagatgaagaaatggccaaaatcgacaggacggcgagggaccagtgtgggagccagccttgggacaataatgcagtctgtgcagacccctgctccctgatccccacacctgacaaagaagatgatgaccgggtttacccaaactcaacgtgcaagcctcggattattgcaccatccagaggctccccgctgcctgtactgagctgggcaaatagagaggaagtctggaaaatcatgttaaacaaggaaaagacatacttaagggatcagcactttcttgagcaacaccctcttctgcagccaaaaatgcgagcaattcttctggattggttaatggaggtgtgtgaagtctataaacttcacagggagaccttttacttggcacaagatttctttgaccggtatatggcgacacaagaaaatgttgtaaaaactcttttacagcttattgggatttcatctttatttattgcagccaaacttgaggaaatctatcctccaaagttgcaccagtttgcgtatgtgacagatggagcttgttcaggagatgaaattctcaccatggaattaatgattatgaaggcccttaagtggcgtttaagtcccctgactattgtgtcctggctgaatgtatacatgcaggttgcatatctaaatgacttacatgaagtgctactgccgcagtatccccagcaaatctttatacagattgcagagctgttggatctctgtgtcctggatgttgactgccttgaatttccttatggtatacttgctgcttcggccttgtatcatttctcgtcatctgaattgatgcaaaaggtttcagggtatcagtggtgcgacatagagaactgtgtcaagtggatggttccatttgccatggttataagggagacggggagctcaaaactgaagcacttcaggggcgtcgctgatgaagatgcacacaacatacagacccacagagacagcttggatttgctggacaaagcccgagcaaagaaagccatgttgtctgaacaaaatagggcttctcctctccccagtgggctcctcaccccgccacagagcggtaagaagcagagcagcgggccggaaatggcgtgaccaccccatccttctccaccaaagacagttgcgcgcctgctccacgttctcttctgtctgttgcagcggaggcgtgcgtttgcttttacagatatctgaatggaagagtgtttcttccacaacagaagtatttctgtggatggcatcaaacagggcaaagtgttttttattgaatgcttataggttttttttaaataagtgggtcaagtacaccagccacctccagacaccagtgcgtgctcccgatgctgctatggaaggtgctacttgacctaagggactcccacaacaacaaaagcttgaagctgtggagggccacggtggcgtggctctcctcgcaggtgttctgggctccgttgtaccaagtggagcaggtggttgcgggcaagcgttgtgcagagcccatagccagctgggcagggggctgccctctccacattatcagttgacagtgtacaatgcctttgatgaactgttttgtaagtgctgctatatctatccattttttaataaagataatactgtttttgagacaaaaaaaa

SEQ ID NO: 22 - DNA, 36 bp, Artificial Sequence
  Synthetic oligonucleotide
gctgcttcggccttgtatcatttctcgtcatctgaa

SEQ ID NO: 23 - DNA, 35 bp, Artificial Sequence
  Synthetic oligonucleotide
aggtttcagggtatcagtggtgcgacatagagaac

SEQ ID NO: 24 - DNA, 35 bp, Artificial Sequence
  Synthetic oligonucleotide
gctcccgatgctgctatggaaggtgctacttgacc

SEQ ID NO: 25 - DNA, 1474 bp, Homo sapiens
  Homo sapiens cyclin-dependent kinase 4 (CDK4), mRNA.
agccctcccagtttccgcgcgcctctttggcagctggtcacatggtgagggtgggggtgagggggcctctctagcttgcggcctgtgtctatggtcgggccctctgcgtccagctgctccggaccgagctcgggtgtatggggccgtaggaaccggctccggggccccgataacgggccgcccccacagcaccccgggctggcgtgagggtctcccttgatctgagaatggctacctctcgatatgagccagtggctgaaattggtgtcggtgcctatgggacagtgtacaaggcccgtgatccccacagtggccactttgtggccctcaagagtgtgagagtccccaatggaggaggaggtggaggaggccttcccatcagcacagttcgtgaggtggctttactgaggcgactggaggcttttgagcatcccaatgttgtccggctgatggacgtctgtgccacatcccgaactgaccgggagatcaaggtaaccctggtgtttgagcatgtagaccaggacctaaggacatatctggacaaggcacccccaccaggcttgccagccgaaacgatcaaggatctgatgcgccagtttctaagaggcctagatttccttcatgccaattgcatcgttcaccgagatctgaagccagagaacattctggtgacaagtggtggaacagtcaagctggctgactttggcctggccagaatctacagctaccagatggcacttacacccgtggttgttacactctggtaccgagctcccgaagttcttctgcagtccacatatgcaacacctgtggacatgtggagtgttggctgtatctttgcagagatgtttcgtcgaaagcctctcttctgtggaaactctgaagccgaccagttgggcaaaatctttgacctgattgggctgcctccagaggatgactggcctcgagatgtatccctgccccgtggagcctttccccccagagggccccgcccagtgcagtcggtggtacctgagatggaggagtcgggagcacagctgctgctggaaatgctgacttttaacccacacaagcgaatctctgcctttcgagctctgcagcactcttatctacataaggatgaaggtaatccggagtgagcaatggagtggctgccatggaaggaagaaaagctgccatttcccttctggacactgagagggcaatctttgcctttatctctgaggctatggagggtcctcctccatctttctacagagattactttgctgccttaatgacattcccctcccacctctccttttgaggcttctccttctccttcccatttctctacactaaggggtatgttccctcttgtccctttccctacctttatatttggggtccttttttatacaggaaaaacaaaacaaagaaataatggtcttttttttttttttaaaaaaaaaaaaaaaaaaaaaaaaaaaaaaaa

SEQ ID NO: 26 - DNA, 35 bp, Artificial Sequence
  Synthetic oligonucleotide
aaggatctgatgcgccagtttctaagaggcctaga

SEQ ID NO: 27 - DNA, 35 bp, Artificial Sequence
  Synthetic oligonucleotide
gatggcacttacacccgtggttgttacactctggt

SEQ ID NO: 28 - DNA, 35 bp, Artificial Sequence
  Synthetic oligonucleotide
taaggatgaaggtaatccggagtgagcaatggagt

SEQ ID NO: 29 - DNA, 3720 bp, Homo sapiens
  Homo sapiens catenin (cadherin-associated protein), beta 1,88kDa (CTNNB1), mRNA.
aggatacagcggcttctgcgcgacttataagagctccttgtgcggcgccattttaagcctctcggtctgtggcagcagcgttggcccggccccgggagcggagagcgaggggaggcggagacggaggaaggtctgaggagcagcttcagtccccgccgagccgccaccgcaggtcgaggacggtcggactcccgcggcgggaggagcctgttcccctgagggtatttgaagtataccatacaactgttttgaaaatccagcgtggacaatggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaaatcatcctttaggtaagaagttttaaaaagccagtttgggtaaaatacttttactctgcctacagaacttcagaaagacttggttggtagggtgggagtggtttaggctatttgtaaatctgccacaaaaacaggtatatactttgaaaggagatgtcttggaacattggaatgttctcagatttctggttgttatgtgatcatgtgtggaagttattaactttaatgttttttgccacagcttttgcaacttaatactcaaatgagtaacatttgctgttttaaacattaatagcagcctttctctctttatacagctgtattgtctgaacttgcattgtgattggcctgtagagttgctgagagggctcgaggggtgggctggtatctcagaaagtgcctgacacactaaccaagctgagtttcctatgggaacaattgaagtaaactttttgttctggtcctttttggtcgaggagtaacaatacaaatggattttgggagtgactcaagaagtgaagaatgcacaagaatggatcacaagatggaatttatcaaaccctagccttgcttgttaaatttttttttttttttttttaagaatatctgtaatggtactgactttgcttgctttgaagtagctctttttttttttttttttttttttttgcagtaactgttttttaagtctctcgtagtgttaagttatagtgaatactgctacagcaatttctaatttttaagaattgagtaatggtgtagaacactaattcataatcactctaattaattgtaatctgaataaagtgtaacaattgtgtagcctttttgtataaaatagacaaatagaaaatggtccaattagtttcctttttaatatgcttaaaataagcaggtggatctatttcatgtttttgatcaaaaactatttgggatatgtatgggtagggtaaatcagtaagaggtgttatttggaaccttgttttggacagtttaccagttgccttttatcccaaagttgttgtaacctgctgtgatacgatgcttcaagagaaaatgcggttataaaaaatggttcagaattaaacttttaattcattcgattg

SEQ ID NO: 30 - DNA, 36 bp, Artificial Sequence
  Synthetic oligonucleotide
cattgtgattggcctgtagagttgctgagagggctc

SEQ ID NO: 31 - DNA, 35 bp, Artificial Sequence
  Synthetic oligonucleotide
tgcctgacacactaaccaagctgagtttcctatgg

SEQ ID NO: 32 - DNA, 36 bp, Artificial Sequence
  Synthetic oligonucleotide
tcccaaagttgttgtaacctgctgtgatacgatgct

SEQ ID NO: 33 - DNA, 4816 bp, Homo sapiens
  Homo sapiens v-erb-b2 erythroblastic leukemia viral oncogene homolog 2, neuro/glioblastoma derived oncogene homolog (avian) (ERBB2), transcript variant 2, mRNA.
gttcccggatttttgtgggcgcctgccccgcccctcgtccccctgctgtgtccatatatcgaggcgatagggttaagggaaggcggacgcctgatgggttaatgagcaaactgaagtgttttccatgatcttttttgagtcgcaattgaagtaccacctcccgagggtgattgcttccccatgcggggtagaacctttgctgtcctgttcaccactctacctccagcacagaatttggcttatgcctactcaatgtgaagatgatgaggatgaaaacctttgtgatgatccacttccacttaatgaatggtggcaaagcaaagctatattcaagaccacatgcaaagctactccctgagcaaagagtcacagataaaacgggggcaccagtagaatggccaggacaaacgcagtgcagcacagagactcagaccctggcagccatgcctgcgcaggcagtgatgagagtgacatgtactgttgtggacatgcacaaaagtgagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcatctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaaccagaaggccaagtccgcagaagccctgatgtgtcctcagggagcagggaaggcctgacttctgctggcatcaagaggtgggagggccctccgaccacttccaggggaacctgccatgccaggaacctgtcctaaggaaccttccttcctgcttgagttcccagatggctggaaggggtccagcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagcta

SEQ ID NO: 34 - DNA, 35 bp, Artificial Sequence
  Synthetic oligonucleotide
cagccttcgacaacctctattactgggaccaggac

SEQ ID NO: 35 - DNA, 35 bp, Artificial Sequence
  Synthetic oligonucleotide
atcctgggtactgaaagccttagggaagctggcct

SEQ ID NO: 36 - DNA, 35 bp, Artificial Sequence
  Synthetic oligonucleotide
gaacagcaatggtgtcagtatccaggctttgtaca

SEQ ID NO: 37 - DNA, 6456 bp, Homo sapiens
  Homo sapiens estrogen receptor 1 (ESR1), mRNA.
gagttgtgcctggagtgatgtttaagccaatgtcagggcaaggcaacagtccctggccgtcctccagcacctttgtaatgcatatgagctcgggagaccagtacttaaagttggaggcccgggagcccaggagctggcggagggcgttcgtcctgggactgcacttgctcccgtcgggtcgcccggcttcaccggacccgcaggctcccggggcagggccggggccagagctcgcgtgtcggcgggacatgcgctgcgtcgcctctaacctcgggctgtgctctttttccaggtggcccgccggtttctgagccttctgccctgcggggacacggtctgcaccctgcccgcggccacggaccatgaccatgaccctccacaccaaagcatccgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctgagagctccctggctcccacacggttcagataatccctgctgcattttaccctcatcatgcaccactttagccaaattctgtctcctgcatacactccggcatgcatccaacaccaatggctttctagatgagtggccattcatttgcttgctcagttcttagtggcacatcttctgtcttctgttgggaacagccaaagggattccaaggctaaatctttgtaacagctctctttcccccttgctatgttactaagcgtgaggattcccgtagctcttcacagctgaactcagtctatgggttggggctcagataactctgtgcatttaagctacttgtagagacccaggcctggagagtagacattttgcctctgataagcactttttaaatggctctaagaataagccacagcaaagaatttaaagtggctcctttaattggtgacttggagaaagctaggtcaagggtttattatagcaccctcttgtattcctatggcaatgcatccttttatgaaagtggtacaccttaaagcttttatatgactgtagcagagtatctggtgattgtcaattcattccccctataggaatacaaggggcacacagggaaggcagatcccctagttggcaagactattttaacttgatacactgcagattcagatgtgctgaaagctctgcctctggctttccggtcatgggttccagttaattcatgcctcccatggacctatggagagcagcaagttgatcttagttaagtctccctatatgagggataagttcctgatttttgtttttatttttgtgttacaaaagaaagccctccctccctgaacttgcagtaaggtcagcttcaggacctgttccagtgggcactgtacttggatcttcccggcgtgtgtgtgccttacacaggggtgaactgttcactgtggtgatgcatgatgagggtaaatggtagttgaaaggagcaggggccctggtgttgcatttagccctggggcatggagctgaacagtacttgtgcaggattgttgtggctactagagaacaagagggaaagtagggcagaaactggatacagttctgaggcacagccagacttgctcagggtggccctgccacaggctgcagctacctaggaacattccttgcagaccccgcattgccctttgggggtgccctgggatccctggggtagtccagctcttcttcatttcccagcgtggccctggttggaagaagcagctgtcacagctgctgtagacagctgtgttcctacaattggcccagcaccctggggcacgggagaagggtggggaccgttgctgtcactactcaggctgactggggcctggtcagattacgtatgcccttggtggtttagagataatccaaaatcagggtttggtttggggaagaaaatcctcccccttcctcccccgccccgttccctaccgcctccactcctgccagctcatttccttcaatttcctttgaacctataggctaaaaaagaaaggctcattccagccacagggcagccttccctgggcctttgcttctctagcacaattatgggttacttcctttttcttaacaaaaaagaatgtttgatttcctctgggtgaccttattgtctgtaattgaaaccctattgagaggtgatgtctgtgttagccaatgacccaggtgagctgctcgggcttctcttggtatgtcttgtttggaaaagtggatttcattcatttctgattgtccagttaagtgatcaccaaaggactgagaatctgggagggcaaaaaaaaaaaaaaagtttttatgtgcacttaaatttggggacaattttatgtatctgtgttaaggatatgtttaagaacataattcttttgttgctgtttgtttaagaagcaccttagtttgtttaagaagcaccttatatagtataatatatatttttttgaaattacattgcttgtttatcagacaattgaatgtagtaattctgttctggatttaatttgactgggttaacatgcaaaaaccaaggaaaaatatttagtttttttttttttttttgtatacttttcaagctaccttgtcatgtatacagtcatttatgcctaaagcctggtgattattcatttaaatgaagatcacatttcatatcaacttttgtatccacagtagacaaaatagcactaatccagatgcctattgttggatattgaatgacagacaatcttatgtagcaaagattatgcctgaaaaggaaaattattcagggcagctaattttgcttttaccaaaatatcagtagtaatatttttggacagtagctaatgggtcagtgggttctttttaatgtttatacttagattttcttttaaaaaaattaaaataaaacaaaaaaaaatttctaggactagacgatgtaataccagctaaagccaaacaattatacagtggaaggttttacattattcatccaatgtgtttctattcatgttaagatactactacatttgaagtgggcagagaacatcagatgattgaaatgttcgcccaggggtctccagcaactttggaaatctctttgtatttttacttgaagtgccactaatggacagcagatattttctggctgatgttggtattgggtgtaggaacatgatttaaaaaaaaactcttgcctctgctttcccccactctgaggcaagttaaaatgtaaaagatgtgatttatctggggggctcaggtatggtggggaagtggattcaggaatctggggaatggcaaatatattaagaagagtattgaaagtatttggaggaaaatggttaattctgggtgtgcaccagggttcagtagagtccacttctgccctggagaccacaaatcaactagctccatttacagccatttctaaaatggcagcttcagttctagagaagaaagaacaacatcagcagtaaagtccatggaatagctagtggtctgtgtttcttttcgccattgcctagcttgccgtaatgattctataatgccatcatgcagcaattatgagaggctaggtcatccaaagagaagaccctatcaatgtaggttgcaaaatctaacccctaaggaagtgcagtctttgatttgatttccctagtaaccttgcagatatgtttaaccaagccatagcccatgccttttgagggctgaacaaataagggacttactgataatttacttttgatcacattaaggtgttctcaccttgaaatcttatacactgaaatggccattgatttaggccactggcttagagtactccttcccctgcatgacactgattacaaatactttcctattcatactttccaattatgagatggactgtgggtactgggagtgatcactaacaccatagtaatgtctaatattcacaggcagatctgcttggggaagctagttatgtgaaaggcaaatagagtcatacagtagctcaaaaggcaaccataattctctttggtgcaggtcttgggagcgtgatctagattacactgcaccattcccaagttaatcccctgaaaacttactctcaactggagcaaatgaactttggtcccaaatatccatcttttcagtagcgttaattatgctctgtttccaactgcatttcctttccaattgaattaaagtgtggcctcgtttttagtcatttaaaattgttttctaagtaattgctgcctctattatggcacttcaattttgcactgtcttttgagattcaagaaaaatttctattcttttttttgcatccaattgtgcctgaacttttaaaatatgtaaatgctgccatgttccaaacccatcgtcagtgtgtgtgtttagagctgtgcaccctagaaacaacatattgtcccatgagcaggtgcctgagacacagacccctttgcattcacagagaggtcattggttatagagacttgaattaataagtgacattatgccagtttctgttctctcacaggtgataaacaatgctttttgtgcactacatactcttcagtgtagagctcttgttttatgggaaaaggctcaaatgccaaattgtgtttgatggattaatatgcccttttgccgatgcatactattactgatgtgactcggttttgtcgcagctttgctttgtttaatgaaacacacttgtaaacctcttttgcactttgaaaaagaatccagcgggatgctcgagcacctgtaaacaattttctcaacctatttgatgttcaaataaagaattaaact

SEQ ID NO: 38 - DNA, 35 bp, Artificial Sequence
  Synthetic oligonucleotide
aggtcttgggagcgtgatctagattacactgcacc

SEQ ID NO: 39 - DNA, 37 bp, Artificial Sequence
  Synthetic oligonucleotide
cccttttgccgatgcatactattactgatgtgactcg

SEQ ID NO: 40 - DNA, 35 bp, Artificial Sequence
  Synthetic oligonucleotide
atccagcgggatgctcgagcacctgtaaacaattt

SEQ ID NO: 41 - DNA, 683 bp, Homo sapiens
  HOST2
agggaagagagagagaccctctcatattgttttatattgttttatattcagtacctcttttaagaaaaagtgacaaggaagtaaaaccaaagacaggcagcctggtgccaggcctgaaaccaggcctgggcctgtctggcctaaacccagtagttaaaaatcaactcataacttagaaactgatgttattcatagattccagacattgtatagaagaacattgtgaaactccctgccctgttctgtttctctctgaccactggtgcatgcagcccctgtcatattccagacattgtatagaagaacattgtgaaactccctgccctgttctgtttctctctgaccactggtgcatgcagcccctgtcatgtaccgcctgcttgctcaaatcaatcacgaccctttcatgtgaaatctttagtgttgtgagcccttaaaagggacagaaattgagtatttggggagcttggatttaaggcagtagcttgctgatgctcccagctgaataaagcccttccttcctacaatttggtgtctgagggttttgtctcctacaatttggtgtctgaggggttttgtctggcggctcgtccagtagcttggctgatgctcccagctgaataaagcccttccttctacaatttggtgtctgaggggttttgtctgcggctcgtcctgctaca

SEQ ID NO: 42 - DNA, 35 bp, Artificial Sequence
  Synthetic oligonucleotide
cgcctgcttgctcaaatcaatcacgaccctttcat

SEQ ID NO: 43 - DNA, 35 bp, Artificial Sequence
  Synthetic oligonucleotide
gcttggatttaaggcagtagcttgctgatgctccc

SEQ ID NO: 44 - DNA, 37 bp, Artificial Sequence
  Synthetic oligonucleotide
tgctcccagctgaataaagcccttccttctacaattt

SEQ ID NO: 45 - DNA, 2230 bp, Homo sapiens
  Homo sapiens hydroxysteroid (17-beta) dehydrogenase 1 (HSD17B1), mRNA.
gggcctcccaaagtgctgggattagaggcatgagccaccgtgcccagcctcaaagcatattttaaaggatagaaataaacagccatatgaagagatacagacagggcggtctggaagggtccagagcaggagcttctatctccatagagttggggttacgtcaccctctgggcacattctgtcagcctccacacgttcagctctcagaagctcccgaaccctgtcctttgggccttttatggagaactccattggctgtccatgactgaagcatggacaactgtgataatgtgattgggcaaaaagggtctgatctaagcccagcaaggccagtccagattctttgggcctttgtgcagcattcctttctccagggtatggggcaaggacccactctggaatgaggatcctacaacccacaatcagattagagtcctgccttgggcagctgaaaagaggacaggagaaggtcagagagacgaaaggctgttttttgaggcctgaggcaccccaacatgacaacgtaagactgtaaccatggtcatgtgagttatgagctaggaaccctggacgaaaccaacacatatacaatcatctcccacctcccaacgcctttactttcacagcctctgcagcaaactgcggtcactataatcgctcctgtggcacagaggcatacccaggggaatctgcccagggggccactctgtgcccacgtgggaacccacacctgcttgtaaagcctcccctccctctgaccagcaaccaggacagtttgttgttccaagcagtgggctcatgtctgttttggctcagaacagggtggggagagcgggccagggacccgcaggaaggcttatccttgagattgcgtgggagacacaacaaggggtgggggcccgcaggcggggcggggcgaagcaggtgatatcaagcccagagccccagcctctccccacagtctcaccatggcccgcaccgtggtgctcatcaccggctgttcctcgggcatcggcctgcacttggccgtacgtctggcttcagatccatcccagagcttcaaagtgtatgccacgttgagggacctgaaaacacagggccggctgtgggaggcggcccgggccctggcatgccctccgggatccctggagacgttgcagctggacgtaagggactcaaaatccgtggccgctgcccgggaacgcgtgactgagggccgcgtggacgtgctggtgtgtaacgcaggcctgggcctgctggggccgctggaggcgctgggggaggacgccgtggcctctgtgctggacgtgaatgtagtagggactgtgcggatgctgcaggccttcctgccagacatgaagaggcgcggttcgggacgcgtgttggtgaccgggagcgtgggaggattgatggggctgcctttcaatgacgtttattgcgccagcaagttcgcgctcgaaggcttatgcgagagtctggcggttctgctgctgccctttggggtccacttgagcctgatcgagtgcggcccagtgcacaccgccttcatggagaaggtgttgggcagcccagaggaggtgctggaccgcacggacatccacaccttccaccgcttctaccaatacctcgcccacagcaagcaagtctttcgcgaggcggcgcagaaccctgaggaggtggcggaggtcttcctcaccgctttgcgcgccccgaagccgaccctgcgctacttcaccaccgagcgcttcctgcccctgctgcggatgcgcctggacgaccccagcggctccaactacgtcaccgccatgcaccgggaagtgttcggcgacgttccggcaaaggccgaggctggggccgaggctgggggcggggccgggcctggggcagaggacgaggccgggcgcagtgcggtgggggaccctgagctcggcgatcctccggccgccccgcagtaaaggcttcctcagccgctgtctcccgcgcccttctttgtcccctgggtctgtgtggtccctggggatggggcggcggtagcagctgtgggtggctaattaagatagatcgcgttagccagttttaccagcgcagctaggcgcgatggctgtcgcctgtaatgccagcgctttgggaggcggaggcaggaggatcgctcaagccccggagttggagaccagccagagcaacacagtgagacccccatctctacaaaaataaagaaaatttaaaaatc

SEQ ID NO: 46 - DNA, 36 bp, Artificial Sequence
  Synthetic oligonucleotide
gacgtgaatgtagtagggactgtgcggatgctgcag

SEQ ID NO: 47 - DNA, 35 bp, Artificial Sequence
  Synthetic oligonucleotide
caagttcgcgctcgaaggcttatgcgagagtctgg

SEQ ID NO: 48 - DNA, 35 bp, Artificial Sequence
  Synthetic oligonucleotide
gatagatcgcgttagccagttttaccagcgcagct

SEQ ID NO: 49 - DNA, 1955 bp, Homo sapiens
  Human insulin-like growth factor binding protein 4 (IGFBP4) mRNA, complete cds.
gtgccctccgccgctcgcccgcgcgcccgcgctccccgcctgcgcccagcgccccgcgcccgcgccccagtcctcgggcggtcatgctgcccctctgcctcgtggccgccctgctgctggccgccgggcccgggccgagcctgggcgacgaagccatccactgcccgccctgctccgaggagaagctggcgcgctgccgcccccccgtgggctgcgaggagctggtgcgagagccgggctgcggctgttgcgccacttgcgccctgggcttggggatgccctgcggggtgtacaccccccgttgcggctcgggcctgcgctgctacccgccccgaggggtggagaagcccctgcacacactgatgcacgggcaaggcgtgtgcatggagctggcggagatcgaggccatccaggaaagcctgcagccctctgacaaggacgagggtgaccaccccaacaacagcttcagcccctgtagcgcccatgaccgcaggtgcctgcagaagcacttcgccaaaattcgagaccggagcaccagtgggggcaagatgaaggtcaatggggcgccccgggaggatgcccggcctgtgccccagggctcctgccagagcgagctgcaccgggcgctggagcggctggccgcttcacagagccgcacccacgaggacctctacatcatccccatccccaactgcgaccgcaacggcaacttccaccccaagcagtgtcacccagctctggatgggcagcgtggcaagtgctggtgtgtggaccggaagacgggggtgaagcttccggggggcctggagccaaagggggagctggactgccaccagctggctgacagctttcgagagtgaggcctgccagcaggccagggactcagcgtcccctgctactcctgtgctctggaggctgcagagctgacccagagtggagtctgagtctgagtcctgtctctgcctgcggcccagaagtttccctcaaatgcgcgtgtgcacgtgtgcgtgtgcgtgcgtgtgtgtgtgtttgtgagcatgggtgtgcccttggggtaagccagagcctggggtgttctctttggtgttacacagcccaagaggactgagactggcacttagcccaagaggtctgagccctggtgtgtttccagatcgatcctggattcactcactcactcattccttcactcatccagccacctaaaaacatttactgaccatgtactacgtgccagctctagttttcagccttgggaggttttattctgacttcctctgattttggcatgtggagacactcctataaggagagttcaagcctgtgggagtagaaaaatctcattcccagagtcagaggagaagagacatgtaccttgaccatcgtccttcctctcaagctagccagagggtgggagcctaaggaagcgtggggtagcagatggagtaatggtcacgaggtccagacccactcccaaagctcagacttgccaggctccctttctcttcttccccaggtccttcctttaggtctggttgttgcaccatctgcttggttggctggcagctgagagccctgctgtgggagagcgaagggggtcaaaggaagacttgaagcacagagggctagggaggtggggtacatttctctgagcagtcagggtgggaagaaagaatgcaagagtggactgaatgtgcctaatggagaagacccacgtgctaggggatgaggggcttcctgggtcctgttccctaccccatttgtggtcacagccatgaagtcaccgggatgaacctatccttccagtggctcgctccctgtagctctgcctccctctccatatctccttcccctacacctccctccccacacctccctactcccctgggcatcttctggcttgactggatggaaggagacttaggaacctaccagttggccatgatgtcttttct

SEQ ID NO: 50 - DNA, 35 bp, Artificial Sequence
  Synthetic oligonucleotide
gaggactgagactggcacttagcccaagaggtctg

SEQ ID NO: 51 - DNA, 35 bp, Artificial Sequence
  Synthetic oligonucleotide
agaagagacatgtaccttgaccatcgtccttcctc

SEQ ID NO: 52 - DNA, 35 bp, Artificial Sequence
  Synthetic oligonucleotide
gccatgaagtcaccgggatgaacctatccttccag

SEQ ID NO: 53 - DNA, 952 bp, Homo sapiens
  Human insulin-like growth factor binding protein 6 (IGFBP6) mRNA, complete cds.
gcagctgcgctgcgactgctctggaaggagaggacggggcacaaaccctgaccatgaccccccacaggctgctgccaccgctgctgctgctgctagctctgctgctcgctgccagcccaggaggcgccttggcgcggtgcccaggctgcgggcaaggggtgcaggcgggttgtccagggggctgcgtggaggaggaggatggggggtcgccagccgagggctgcgcggaagctgagggctgtctcaggagggaggggcaggagtgcggggtctacacccctaactgcgccccaggactgcagtgccatccgcccaaggacgacgaggcgcctttgcgggcgctgctgctcggccgaggccgctgccttccggcccgcgcgcctgctgttgcagaggagaatcctaaggagagtaaaccccaagcaggcactgcccgcccacaggatgtgaaccgcagagaccaacagaggaatccaggcacctctaccacgccctcccagcccaattctgcgggtgtccaagacactgagatgggcccatgccgtagacatctggactcagtgctgcagcaactccagactgaggtctaccgaggggctcaaacactctacgtgcccaattgtgaccatcgaggcttctaccggaagcggcagtgccgctcctcccaggggcagcgccgaggtccctgctggtgtgtggatcggatgggcaagtccctgccagggtctccagatggcaatggaagctcctcctgccccactgggagtagcggctaaagctgggggatagaggggctgcagggccactggaaggaacatggagctgtcatcactcaacaaaaaaccgaggccctcaatccaccttcaggccccgccccatgggcccctcaccgctggttggaaagagtgttggtgttggctggggtgtcaataaagctgtgcttggggtcaaa

SEQ ID NO: 54 - DNA, 35 bp, Artificial Sequence
  Synthetic oligonucleotide
atgtgaaccgcagagaccaacagaggaatccaggc

SEQ ID NO: 55 - DNA, 35 bp, Artificial Sequence
  Synthetic oligonucleotide
agatgggcccatgccgtagacatctggactcagtg

SEQ ID NO: 56 - DNA, 35 bp, Artificial Sequence
  Synthetic oligonucleotide
tacgtgcccaattgtgaccatcgaggcttctaccg

SEQ ID NO: 57 - DNA, 1424 bp, Homo sapiens
  Homo sapiens inhibin, alpha (INHA), mRNA.
gaaggactggggaagactggatgagaagggtagaagagggtgggtgtgggatggggaggggagagtggaaaggccctgggcagaccctggcagaaggggcacggggcagggtgtgagttccccactagcagggccaggtgagctatggtgctgcacctactgctcttcttgctgctgaccccacagggtgggcacagctgccaggggctggagctggcccgggaacttgttctggccaaggtgagggccctgttcttggatgccttggggccccccgcggtgaccagggaaggtggggaccctggagtcaggcggctgccccgaagacatgccctggggggcttcacacacaggggctctgagcccgaggaagaggaggatgtctcccaagccatccttttcccagccacagatgccagctgtgaggacaagtcagctgccagagggctggcccaggaggctgaggagggcctcttcagatacatgttccggccatcccagcatacacgcagccgccaggtgacttcagcccagctgtggttccacaccgggctggacaggcagggcacagcagcctccaatagctctgagcccctgctaggcctgctggcactgtcaccgggaggacccgtggctgtgcccatgtctttgggccatgctccccctcactgggccgtgctgcacctggccacctctgctctctctctgctgacccaccccgtcctggtgctgctgctgcgctgtcccctctgtacctgctcagcccggcctgaggccacgcccttcctggtggcccacactcggaccagaccacccagtggaggggagagagcccgacgctcaactcccctgatgtcctggccttggtctccctctgctctgcgcctgctgcagaggcctccggaggaaccggctgcccatgccaactgccacagagtagcactgaacatctccttccaggagctgggctgggaacggtggatcgtgtaccctcccagtttcatcttccactactgtcatggtggttgtgggctgcacatcccaccaaacctgtcccttccagtccctggggctccccctaccccagcccagccctactccttgctgccaggggcccagccctgctgtgctgctctcccagggaccatgaggcccctacatgtccgcaccacctcggatggaggttactctttcaagtatgagacagtgcccaaccttctcacgcagcactgtgcttgtatctaagggtggggggtcttccttcttaatcccatggctggtggccacgcccccaccatcatcagctgggaggaaaggcagagttgggaaatagatggctcccactcctccctcctttcacttctctgcctatgggctaccctccccaccccacttctatctcaataaagaacacagtgcatatg

SEQ ID NO: 58 - DNA, 35 bp, Artificial Sequence
  Synthetic oligonucleotide
cctcttcagatacatgttccggccatcccagcata

SEQ ID NO: 59 - DNA, 35 bp, Artificial Sequence
  Synthetic oligonucleotide
tgccaactgccacagagtagcactgaacatctcct

SEQ ID NO: 60 - DNA, 37 bp, Artificial Sequence
  Synthetic oligonucleotide
cggatggaggttactctttcaagtatgagacagtgcc

SEQ ID NO: 61 - DNA, 1753 bp, Homo sapiens
  Homo sapiens keratin 7 (KRT7), mRNA.
cagccccgcccctacctgtggaagcccagccgcccgctcccgcggataaaaggcgcggagtgtccccgaggtcagcgagtgcgcgctcctcctcgcccgccgctaggtccatcccggcccagccaccatgtccatccacttcagctccccggtattcacctcgcgctcagccgccttctcgggccgcggcgcccaggtgcgcctgagctccgctcgccccggcggccttggcagcagcagcctctacggcctcggcgcctcacggccgcgcgtggccgtgcgctctgcctatgggggcccggtgggcgccggcatccgcgaggtcaccattaaccagagcctgctggccccgctgcggctggacgccgacccctccctccagcgggtgcgccaggaggagagcgagcagatcaagaccctcaacaacaagtttgcctccttcatcgacaaggtgcggtttctggagcagcagaacaagctgctggagaccaagtggacgctgctgcaggagcagaagtcggccaagagcagccgcctcccagacatctttgaggcccagattgctggccttcggggtcagcttgaggcactgcaggtggatgggggccgcctggaggcggagctgcggagcatgcaggatgtggtggaggacttcaagaataagtacgaagatgaaattaaccaccgcacagctgctgagaatgagtttgtggtgctgaagaaggatgtggatgctgcctacatgagcaaggtggagctggaggccaaggtggatgccctgaatgatgagatcaacttcctcaggaccctcaatgagacggagttgacagagctgcagtcccagatctccgacacatctgtggtgctgtccatggacaacagtcgctccctggacctggacggcatcatcgctgaggtcaaggcgcagtatgaggagatggccaaatgcagccgggctgaggctgaagcctggtaccagaccaagtttgagaccctccaggcccaggctgggaagcatggggacgacctccggaatacccggaatgagatttcagagatgaaccgggccatccagaggctgcaggctgagatcgacaacatcaagaaccagcgtgccaagttggaggccgccattgccgaggctgaggagcgtggggagctggcgctcaaggatgctcgtgccaagcaggaggagctggaagccgccctgcagcggggcaagcaggatatggcacggcagctgcgtgagtaccaggaactcatgagcgtgaagctggccctggacatcgagatcgccacctaccgcaagctgctggagggcgaggagagccggttggctggagatggagtgggagccgtgaatatctctgtgatgaattccactggtggcagtagcagtggcggtggcattgggctgaccctcgggggaaccatgggcagcaatgccctgagcttctccagcagtgcgggtcctgggctcctgaaggcttattccatccggaccgcatccgccagtcgcaggagtgcccgcgactgagccgcctcccaccactccactcctccagccaccacccacaatcacaagaagattcccacccctgcctcccatgcctggtcccaagacagtgagacagtctggaaagtgatgtcagaatagcttccaataaagcagcctcattctgaggcctgagtgatccacgtgaaaaaaaaaaaaaaaaaaaaaaaaaaaaaaaaaaaaaaaaaaaaaaaaaaa

SEQ ID NO: 62 - DNA, 36 bp, Artificial Sequence
  Synthetic oligonucleotide
ttcctcaggaccctcaatgagacggagttgacagag

SEQ ID NO: 63 - DNA, 35 bp, Artificial Sequence
  Synthetic oligonucleotide
tcgacaacatcaagaaccagcgtgccaagttggag

SEQ ID NO: 64 - DNA, 35 bp, Artificial Sequence
  Synthetic oligonucleotide
gcctggtcccaagacagtgagacagtctggaaagt

SEQ ID NO: 65 - DNA, 9534 bp, Homo sapiens
  H.sapiens mRNA for laminin M chain (merosin).
cagcgactcctctggctcccgagaagtggatccggtcgcggccactacgatgccgggagccgccggggtcctcctccttctgctgctctccggaggcctcgggggcgtacaggcgcagcggccgcagcagcagcggcagtcacaggcacatcagcaaagaggtttattccctgctgtcctgaatcttgcttctaatgctcttatcacgaccaatgcaacatgtggagaaaaaggacctgaaatgtactgcaaattggtagaacatgtccctgggcagcctgtgaggaacccgcagtgtcgaatctgcaatcaaaacagcagcaatccaaaccagagacacccgattacaaatgctattgatggaaagaacacttggtggcagagtcccagtattaagaatggaatcgaataccattatgtgacaattacactggatttacagcaggtgttccagatcgcgtatgtgattgtgaaggcagctaactccccccggcctggaaactggattttggaacgctctcttgatgatgttgaatacaagccctggcagtatcatgctgtgacagacacggagtgcctaacgctttacaatatttatccccgcactgggccaccgtcatatgccaaagatgatgaggtcatctgcacttcattttactccaagatacaccccttagaaaatggagagattcacatctctttaatcaatgggagaccaagtgccgatgatccttctccagaactgctagaatttacctccgctcgctatattcgcctgagatttcagaggatccgcacactgaatgctgacttgatgatgtttgctcacaaagacccaagagaaattgaccccattgtcaccagaagatattactactcggtcaaggatatttcagttggagggatgtgcatctgctatggtcatgccagggcttgtccacttgatccagcgacaaataaatctcgctgtgagtgtgagcataacacatgtggcgatagctgtgatcagtgctgtccaggattccatcagaaaccctggagagctggaacttttctaactaaaactgaatgtgaagcatgcaattgtcatggaaaagctgaagaatgctattatgatgaaaatgttgccagaagaaatctgagtttgaatatacgtggaaagtacattggagggggtgtctgcattaattgtacccaaaacactgctggtataaactgcgagacatgtacagatggcttcttcagacccaaaggggtatctccaaattatccaaggccatgccagccatgtcattgcgatccaattggttccttaaatgaagtctgtgtcaaggatgagaaacatgctcgacgaggtttggcacctggatcctgtcattgcaaaactggttttggaggtgtgagctgtgatcggtgtgccaggggctacactggctacccggactgcaaagcctgtaactgcagtgggttagggagcaaaaatgaggatccttgttttggcccctgtatctgcaaggaaaatgttgaaggaggagactgtagtcgttgcaaatccggcttcttcaatttgcaagaggataattggaaaggctgcgatgagtgtttctgttcaggggtttcaaacagatgtcagagttcctactggacctatggcaaaatacaagatatgagtggctggtatctgactgaccttcctggccgcattcgagtggctccccagcaggacgacttggactcacctcagcagatcagcatcagtaacgcggaggcccggcaagccctgccgcacagctactactggagcgcgccggctccctatctgggaaacaaactcccagcagtaggaggacagttgacatttaccatatcatatgaccttgaagaagaggaagaagatacagaacgtgttctccagcttatgattatcttagagggtaatgacttgagcatcagcacagcccaagatgaggtgtacctgcacccatctgaagaacatactaatgtattgttacttaaagaagaatcatttaccatacatggcacacattttccagtccgtagaaaggaatttatgacagtgcttgcgaatttgaagagagtcctcctacaaatcacatacagctttgggatggatgccatcttcaggttgagctctgttaaccttgaatccgctgtctcctatcctactgatggaagcattgcagcagctgtagaagtgtgtcagtgcccaccagggtatactggctcctcttgtgaatcttgttggcctaggcacaggcgagttaacggcactatttttggtggcatctgtgagccatgtcagtgctttggtcatgcggagtcctgtgatgacgtcactggagaatgcctgaactgtaaggatcacacaggtggcccatattgtgataaatgtcttcctggtttctatggcgagcctactaaaggaacctctgaagactgtcaaccctgtgcctgtccactcaatatcccatccaataactttagcccaacgtgccatttagaccggagtcttggattgatctgtgatggatgccctgtcgggtacacaggaccacgctgtgagaggtgtgcagaaggctattttggacaaccctctgtacctggaggatcatgtcagccatgccaatgcaatgacaaccttgacttctccatccctggcagctgtgacagcttgtctggctcctgtctgatatgtaaaccaggtacaacaggccggtactgtgagctctgtgctgatggatattttggagatgcagttgatgcgaagaactgtcagccctgtcgctgtaatgccggtggctctttctctgaggtttgccacagtcaaactggacagtgtgagtgcagagccaacgttcagggtcagagatgtgacaaatgcaaggctgggacctttggcctacaatcagcaaggggctgtgttccctgcaactgcaattcttttgggtctaagtcattcgactgtgaagagagtggacaatgttggtgccaacctggagtcacagggaagaaatgtgaccgctgtgcccacggctatttcaacttccaagaaggaggctgcacagcttgtgaatgttctcatctgggtaataattgtgacccaaagactgggcgatgcatttgcccacccaataccattggagagaaatgttctaaatgtgcacccaatacctggggccacagcattaccactggttgtaaggcttgtaactgcagcacagtgggatccttggatttccaatgcaatgtaaatacaggccaatgcaactgtcatccaaaattctctggtgcaaaatgtacagagtgcagtcgaggtcactggaactaccctcgctgcaatctctgtgactgcttcctccctgggacagatgccacaacctgtgattcagagactaaaaaatgctcctgtagtgatcaaactgggcagtgcacttgtaaggtgaatgtggaaggcatccactgtgacagatgccggcctggcaaattcggactcgatgccaagaatccacttggctgcagcagctgctattgcttcggcactactacccagtgctctgaagcaaaaggactgatccggacgtgggtgactctgaaggctgagcagaccattctacccctggtagatgaggctctgcagcacacgaccaccaagggcattgtttttcaacatccagagattgttgcccacatggacctgatgagagaagatctccatttggaacctttttattggaaacttccagaacaatttgaaggaaagaagttgatggcctatgggggcaaactcaagtatgcaatctatttcgaggctcgggaagaaacaggtttctctacatataatcctcaagtgatcattcgaggtgggacacctactcatgctagaattatcgtcaggcatatggctgctcctctgattggccaattgacaaggcatgaaattgaaatgacagagaaagaatggaaatattatggggatgatcctcgagtccatagaactgtgacccgagaagacttcttggatatactatatgatattcattacattcttatcaaagctacttatggaaatttcatgcgacaaagcaggatttctgaaatctcaatggaggtagctgaacaaggacgtggaacaacaatgactcctccagctgacttgattgaaaaatgtgattgtcccctgggctattctggcctgtcctgtgaggcatgcttgccgggattttatcgactgcgttctcaaccaggtggccgcacccctggaccaaccctgggcacctgtgttccatgtcaatgtaatggacacagcagcctgtgtgaccctgaaacatcgatatgccagaattgtcaacatcacactgctggtgacttctgtgaacgatgtgctcttggatactatggaattgtcaagggattgccaaatgactgtcagcaatgtgcctgccctctgatttcttccagtaacaatttcagcccctcttgtgtcgcagaaggacttgacgactaccgctgcacggcttgtccacggggatatgaaggccagtactgtgaaaggtgtgcccctggctatactggcagtccaggcaaccctggaggctcctgccaagaatgtgagtgtgatccctatggctcactgcctgtgccctgtgaccctgtcacaggattctgcacgtgccgacctggagccacgggaaggaagtgtgacggctgcaagcactggcatgcacgcgagggctgggagtgtgttttttgtggagatgagtgcactggccttcttctcggtgacttggctcgcctggagcagatggtcatgagcatcaacctcactggtccgctgcctgcgccatataaaatgctgtatggtcttgaaaatatgactcaggagctaaagcacttgctgtcacctcagcgggccccagagaggcttattcagctggcagagggcaatctgaatacactcgtgaccgaaatgaacgagctgctgaccagggctaccaaagtgacagcagatggcgagcagaccggacaggatgctgagaggaccaacacaagagcaaagtccctgggagaattcattaaggagcttgcccgggatgcagaagctgtaaatgaaaaagctataaaactaaatgaaactctaggaactcgagacgaggcctttgagagaaatttggaagggcttcagaaagagattgaccagatgattaaagaactgaggaggaaaaatctagagacacaaaaggaaattgctgaagatgagttggtagctgcagaagcccttctgaaaaaagtgaagaagctgtttggagagtcccggggggaaaatgaagaaatggagaaggatctccgggaaaaactggctgactacaaaaacaaagttgatgatgcttgggaccttttgagagaagccacagataaaatcagagaagctaatcgcctatttgcagtaaatcagaaaaacatgactgcattggagaaaaagaaggaggctgttgagagcggcaaacgacaaattgagaacactttaaaagaaggcaatgacatactcgatgaagccaaccgtcttgcagatgaaatcaactccatcatagactatgttgaagacatccaaactaaattgccacctatgtctgaggagcttaatgataaaatagatgacctctcccaagaaataaaggacaggaagcttgctgagaaggtgtcccaggctgagagccacgcagctcagttgaatgactcatctgctgtccttgatggaatccttgatgaggctaaaaacatctccttcaatgccactgcagccttcaaagcttacagcaatattaaggactatattgatgaagctgagaaagttgccaaagaagccaaagatcttgcacatgaagctacaaaactggcaacaggtcctcggggtttattaaaggaagatgccaaaggctgtcttcagaaaagcttcaggattcttaacgaagccaagaagttagcaaatgatgtaaaagaaaatgaagaccatctaaatggcttaaaaaccaggatagaaaatgctgatgctagaaatggggatctcttgagaactttgaatgacactttgggaaagttatcagctattccaaatgatacagctgctaaactgcaagctgttaaggacaaagccagacaagccaacgacacagctaaagatgtactggcacagattacagagctccaccagaacctcgatggcctgaagaagaattacaataaactagcagacagcgtcgccaaaacgaatgctgtggttaaagatccttccaagaacaaaatcattgccgatgcagatgccactgtcaaaaatttagaacaggaagctgaccggctaatagataaactcaaacccatcaaggaacttgaggataacctaaagaaaaacatctctgagataaaggaattgataaaccaagctcggaaacaagccaattctatcaaagtatctgtgtcttcaggaggtgactgcattcgaacatacaaaccagaaatcaagaaaggaagttacaataatattgttgtcaacgtaaagacagctgttgctgataacctcctcttttatcttggaagtgccaaatttattgactttctggctatagaaatgcgtaaaggcaaagtcagcttcctctgggatgttggatctggagttggacgtgtagagtacccagatttgactattgatgactcatattggtaccgtatcgtagcatcaagaactgggagaaatggaactatttctgtgagagccctggatggacccaaagccagcattgtgcccagcacacaccattcgacgtctcctccagggtacacgattctagatgtggatgcaaatgcaatgctgtttgttggtggcctgactgggaaattaaagaaggctgatgctgtacgtgtgattacattcactggctgcatgggagaaacatactttgacaacaaacctataggtttgtggaatttccgagaaaaagaaggtgactgcaaaggatgcactgtcagtcctcaggtggaagatagtgaggggactattcaatttgatggagaaggttatgcattggtcagccgtcccattcgctggtaccccaacatctccactgtcatgttcaagttcagaacattttcttcgagtgctcttctgatgtatcttgccacacgagacctgagagatttcatgagtgtggagctcactgatgggcacataaaagtcagttacgatctgggctcaggaatggcttccgttgtcagcaatcaaaaccataatgatgggaaatggaaatcattcactctgtcaagaattcaaaaacaagccaatatatcaattgtagatatagatactaatcaggaggagaatatagcaacttcgtcttctggaaacaactttggtcttgacttgaaagcagatgacaaaatatattttggtggcctgccaacgctgagaaacttgagtatgaaagcaaggccagaagtaaatctgaagaaatattccggctgcctcaaagatattgaaatttcaagaactccgtacaatatactcagtagtcccgattatgttggtgttaccaaaggatgttccctggagaatgtttacacagttagctttcctaagcctggttttgtggagctctcccctgtgccaattgatgtaggaacagaaatcaacctgtcattcagcaccaagaatgagtccggcatcattcttttgggaagtggagggacaccagcaccacctaggagaaaacgaaggcagactggacaggcctattatgtaatactcctcaacaggggccgtctggaagtgcatctctccacaggggcacgaacaatgaggaaaattgtcatcagaccagagccgaatctgtttcatgatggaagagaacattccgttcatgtagagcgaactagaggcatctttacagttcaagtggatgaaaacagaagatacatgcaaaacctgacagttgaacagcctatcgaagttaaaaagcttttcgttgggggtgctccacctgaatttcaaccttccccactcagaaatattcctccttttgaaggctgcatatggaatcttgttattaactctgtccccatggactttgcaaggcctgtgtccttcaaaaatgctgacattggtcgctgtgcccatcagaaactccgtgaagatgaagatggagcagctccagctgaaatagttatccagcctgagccagttcccaccccagcctttcctacgcccaccccagttctgacacatggtccttgtgctgcagaatcagaaccagctcttttgatagggagcaagcagttcgggctttcaagaaacagtcacattgcaattgcatttgatgacaccaaagttaaaaaccgtctcacaattgagttggaagtaagaaccgaagctgaatccggcttgcttttttacatggctgcgatcaatcatgctgattttgcaacagttcagctgagaaatggattgccctacttcagctatgacttggggagtggggacacccacaccatgatccccaccaaaatcaatgatggccagtggcacaagattaagataatgagaagtaagcaagaaggaattctttatgtagatggggcttccaacagaaccatcagtcccaaaaaagccgacatcctggatgtcgtgggaatgctgtatgttggtgggttacccatcaactacactacccgaagaattggtccagtgacctatagcattgatggctgcgtcaggaatctccacatggcagaggcccctgccgatctggaacaacccacctccagcttccatgttgggacatgttttgcaaatgctcagaggggaacatattttgacggaaccggttttgccaaagcagttggtggattcaaagtgggattggaccttcttgtagaatttgaattcgcgacaactacaacgactggagttcttctggggatcagtagtcaaaaaatggatggaatgggtattgaaatgattgatgaaaagttgatgtttcatgtggacaatggtgcgggcagattcactgctgtctatgatgctggggttccagggcatttgtgtgatggacaatggcataaagtcactgccaacaagatcaaacaccgcattgagctcacagtcgatgggaaccaggtggaagcccaaagcccaaacccagcatctacatcagctgacacaaatgaccctgtgtttgttggaggcttcccagatgacctcaagcagtttggcctaacaaccagtattccgttccgaggttgcatcagatccctgaagctcaccaaaggcacagcaagccactggaggttaattttgccaaggccctggaactgaggggcgttcaacctgtatcatgcccagccaactaataaaaataagtgtaaccccaggaagagtctgtcaaaacaagtatatcaagtaaaacaaacaaatatattttacctatatatgttaattaaactaatttgtgcatgtacatagaattc

SEQ ID NO: 66 - DNA, 35 bp, Artificial Sequence
  Synthetic oligonucleotide
gggaatgctgtatgttggtgggttacccatcaact

SEQ ID NO: 67 - DNA, 35 bp, Artificial Sequence
  Synthetic oligonucleotide
tatagcattgatggctgcgtcaggaatctccacat

SEQ ID NO: 68 - DNA, 35 bp, Artificial Sequence
  Synthetic oligonucleotide
gcagtttggcctaacaaccagtattccgttccgag

SEQ ID NO: 69 - DNA, 3546 bp, Homo sapiens
  Homo sapiens matrix metallopeptidase 2 (gelatinase A, 72kDa gelatinase, 72kDa type IV collagenase) (MMP2), mRNA.
gcggctgccctcccttgtttccgctgcatccagacttcctcaggcggtggctggaggctgcgcatctggggctttaaacatacaaagggattgccaggacctgcggcggcggcggcggcggcgggggctggggcgcgggggccggaccatgagccgctgagccgggcaaaccccaggccaccgagccagcggaccctcggagcgcagccctgcgccgcggagcaggctccaaccaggcggcgaggcggccacacgcaccgagccagcgacccccgggcgacgcgcggggccagggagcgctacgatggaggcgctaatggcccggggcgcgctcacgggtcccctgagggcgctctgtctcctgggctgcctgctgagccacgccgccgccgcgccgtcgcccatcatcaagttccccggcgatgtcgcccccaaaacggacaaagagttggcagtgcaatacctgaacaccttctatggctgccccaaggagagctgcaacctgtttgtgctgaaggacacactaaagaagatgcagaagttctttggactgccccagacaggtgatcttgaccagaataccatcgagaccatgcggaagccacgctgcggcaacccagatgtggccaactacaacttcttccctcgcaagcccaagtgggacaagaaccagatcacatacaggatcattggctacacacctgatctggacccagagacagtggatgatgcctttgctcgtgccttccaagtctggagcgatgtgaccccactgcggttttctcgaatccatgatggagaggcagacatcatgatcaactttggccgctgggagcatggcgatggatacccctttgacggtaaggacggactcctggctcatgccttcgccccaggcactggtgttgggggagactcccattttgatgacgatgagctatggaccttgggagaaggccaagtggtccgtgtgaagtatgggaacgccgatggggagtactgcaagttccccttcttgttcaatggcaaggagtacaacagctgcactgataccggccgcagcgatggcttcctctggtgctccaccacctacaactttgagaaggatggcaagtacggcttctgtccccatgaagccctgttcaccatgggcggcaacgctgaaggacagccctgcaagtttccattccgcttccagggcacatcctatgacagctgcaccactgagggccgcacggatggctaccgctggtgcggcaccactgaggactacgaccgcgacaagaagtatggcttctgccctgagaccgccatgtccactgttggtgggaactcagaaggtgccccctgtgtcttccccttcactttcctgggcaacaaatatgagagctgcaccagcgccggccgcagtgacggaaagatgtggtgtgcgaccacagccaactacgatgatgaccgcaagtggggcttctgccctgaccaagggtacagcctgttcctcgtggcagcccacgagtttggccacgccatggggctggagcactcccaagaccctggggccctgatggcacccatttacacctacaccaagaacttccgtctgtcccaggatgacatcaagggcattcaggagctctatggggcctctcctgacattgaccttggcaccggccccacccccacgctgggccctgtcactcctgagatctgcaaacaggacattgtatttgatggcatcgctcagatccgtggtgagatcttcttcttcaaggaccggttcatttggcggactgtgacgccacgtgacaagcccatggggcccctgctggtggccacattctggcctgagctcccggaaaagattgatgcggtatacgaggccccacaggaggagaaggctgtgttctttgcagggaatgaatactggatctactcagccagcaccctggagcgagggtaccccaagccactgaccagcctgggactgccccctgatgtccagcgagtggatgccgcctttaactggagcaaaaacaagaagacatacatctttgctggagacaaattctggagatacaatgaggtgaagaagaaaatggatcctggcttccccaagctcatcgcagatgcctggaatgccatccccgataacctggatgccgtcgtggacctgcagggcggcggtcacagctacttcttcaagggtgcctattacctgaagctggagaaccaaagtctgaagagcgtgaagtttggaagcatcaaatccgactggctaggctgctgagctggccctggctcccacaggcccttcctctccactgccttcgatacaccgggcctggagaactagagaaggacccggaggggcctggcagccgtgccttcagctctacagctaatcagcattctcactcctacctggtaatttaagattccagagagtggctcctcccggtgcccaagaatagatgctgactgtactcctcccaggcgccccttccccctccaatcccaccaaccctcagagccacccctaaagagatactttgatattttcaacgcagccctgctttgggctgccctggtgctgccacacttcaggctcttctcctttcacaaccttctgtggctcacagaacccttggagccaatggagactgtctcaagagggcactggtggcccgacagcctggcacagggcagtgggacagggcatggccaggtggccactccagacccctggcttttcactgctggctgccttagaacctttcttacattagcagtttgctttgtatgcactttgtttttttctttgggtcttgttttttttttccacttagaaattgcatttcctgacagaaggactcaggttgtctgaagtcactgcacagtgcatctcagcccacatagtgatggttcccctgttcactctacttagcatgtccctaccgagtctcttctccactggatggaggaaaaccaagccgtggcttcccgctcagccctccctgcccctcccttcaaccattccccatgggaaatgtcaacaagtatgaataaagacacctactgagtggccgtgtttgccatctgttttagcagagcctagacaagggccacagacccagccagaagcggaaacttaaaaagtccgaatctctgctccctgcagggcacaggtgatggtgtctgctggaaaggtcagagcttccaaagtaaacagcaagagaacctcagggagagtaagctctagtccctctgtcctgtagaaagagccctgaagaatcagcaattttgttgctttattgtggcatctgttcgaggtttgcttcctctttaagtctgtttcttcattagcaatcatatcagttttaatgctactactaacaatgaacagtaacaataatatccccctcaattaatagagtgctttctatgtgcaaggcacttttcacgtgtcacctattttaacctttccaaccacataaataaaaaaggccattattagttgaatcttattgatgaagagaaaaaaaaaaa

SEQ ID NO: 70 - DNA, 35 bp, Artificial Sequence
  Synthetic oligonucleotide
cacagaacccttggagccaatggagactgtctcaa

SEQ ID NO: 71 - DNA, 35 bp, Artificial Sequence
  Synthetic oligonucleotide
acagaaggactcaggttgtctgaagtcactgcaca

SEQ ID NO: 72 - DNA, 35 bp, Artificial Sequence
  Synthetic oligonucleotide
tctatgtgcaaggcacttttcacgtgtcacctatt

SEQ ID NO: 73 - DNA, 931 bp, Homo sapiens
  Homo sapiens TIMP metallopeptidase inhibitor 1 (TIMP1), mRNA.
tttcgtcggcccgccccttggcttctgcactgatggtgggtggatgagtaatgcatccaggaagcctggaggcctgtggtttccgcacccgctgccacccccgcccctagcgtggacatttatcctctagcgctcaggccctgccgccatcgccgcagatccagcgcccagagagacaccagagaacccaccatggccccctttgagcccctggcttctggcatcctgttgttgctgtggctgatagcccccagcagggcctgcacctgtgtcccaccccacccacagacggccttctgcaattccgacctcgtcatcagggccaagttcgtggggacaccagaagtcaaccagaccaccttataccagcgttatgagatcaagatgaccaagatgtataaagggttccaagccttaggggatgccgctgacatccggttcgtctacacccccgccatggagagtgtctgcggatacttccacaggtcccacaaccgcagcgaggagtttctcattgctggaaaactgcaggatggactcttgcacatcactacctgcagttttgtggctccctggaacagcctgagcttagctcagcgccggggcttcaccaagacctacactgttggctgtgaggaatgcacagtgtttccctgtttatccatcccctgcaaactgcagagtggcactcattgcttgtggacggaccagctcctccaaggctctgaaaagggcttccagtcccgtcaccttgcctgcctgcctcgggagccagggctgtgcacctggcagtccctgcggtcccagatagcctgaatcctgcccggagtggaagctgaagcctgcacagtgtccaccctgttcccactcccatctttcttccggacaatgaaataaagagttaccacccagcagaaaaaaaaaaaaaaaa

SEQ ID NO: 74 - DNA, 35 bp, Artificial Sequence
  Synthetic oligonucleotide
ccttctgcaattccgacctcgtcatcagggccaag

SEQ ID NO: 75 - DNA, 35 bp, Artificial Sequence
  Synthetic oligonucleotide
agtcaaccagaccaccttataccagcgttatgaga

SEQ ID NO: 76 - DNA, 35 bp, Artificial Sequence
  Synthetic oligonucleotide
atggagagtgtctgcggatacttccacaggtccca